(19) United States
(12) Patent Application Publication
(10) Pub. No.: US 2018/0028552 A1
(43) Pub. Date: Feb. 1, 2018

(54) COMBINATION THERAPY FOR TREATING CANCER

(71) Applicants: Epizyme, Inc., Cambridge, MA (US); Celgene Corporation, Summit, NJ (US)

(72) Inventors: Christine Klaus, Waban, MA (US); Maria Alejandra Raimondi, Jamaica Plain, MA (US); Scott Richard Daigle, Newburyport, MA (US); Roy MacFarlane Pollock, Medford, MA (US); Vivek Chopra, San Francisco, CA (US)

(21) Appl. No.: 15/547,427

(22) PCT Filed: Feb. 1, 2016
(86) PCT No.: PCT/US2016/016007

§ 371 Date: Jul. 28, 2017

Related U.S. Application Data
(60) Provisional application No. 62/110,208, filed on Jan. 30, 2015.
(60) Provisional application No. 62/149,022, filed on Apr. 17, 2015.

(51) Int. Cl. A61K 31/7076; A61K 31/706

(52) U.S. Cl. CPC A61K 31/7076; A61K 31/706

(57) ABSTRACT

The present disclosure relates to a combination of an inhibitor of human histone methyltransferase DOT1L, such as EPZ-5676 or salts thereof, and one or more DNMT inhibitors, and methods of combination therapy for administering to subjects in need thereof for the treatment of cancer.

### RELATED APPLICATIONS

This application claims the benefit of and priority to U.S. Provisional Application Nos. 62/110,208, filed Jan. 30, 2015 and 62/149,022 filed Apr. 17, 2015, the contents of each of which are hereby incorporated by reference in their entireties.

### FIELD OF DISCLOSURE

This disclosure relates to a combination of an inhibitor of human histone methyltransferase DOT1L and a DNA methyltransferase (DNMT) inhibitor, particularly a DNMT inhibitor anticancer agent, and methods of combination therapy for treating cancer.

### BACKGROUND

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., DOT1L), many of which are associated with specific genetic alterations that can cause human disease.

Disease-associated chromatin-modifying enzymes (e.g., DOT1L) play a role in diseases such as proliferative disorders, metabolic disorders, and blood disorders. Thus, there is a need for the development of compositions that are capable of modulating the activity of DOT1L.

### SUMMARY

In one aspect, this present disclosure features a combination of a DNA methyltransferase (DNMT) inhibitor and Compound A2 (also called “Cpd A2” or “5676” or “EPZ-5676” or “pinometostat”), a DOT1L inhibitor, having the formula:

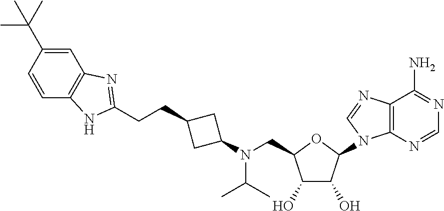 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or a pharmaceutically acceptable salt, polymorph, solvate, or stereoisomer thereof. In some embodiments, the DNMT inhibitor is selected from Azacitidine (also known as 5-azacytidine or Vidaza, or called “AZA” herein), Decitabine (also known as Dacogen or 5-aza-2′-deoxycytidine), and Zebularine (1-(β-D-ribofuranosyl)-1,2-dihydropyrimidin-2-one), or functional analogs, derivatives, prodrugs, and metabolites thereof.

In some embodiments, the DNMT inhibitor and Compound A2 are formulated in the same formulation. In other embodiments, the DNMT inhibitor and Compound A2 are formulated in separate formulations and are administered simultaneously, sequentially or in alternation.

In one aspect, the present disclosure provides a method of treating or alleviating a symptom of a disease by administering to a subject in need thereof a therapeutically effective amount of a combination described herein. The disease is cancer or a precancerous condition. Alternatively, the disease can be influenced by modulating the methylation status of histones or other proteins. The methylation status is mediated at least in part by the activity of DOT1L.

In one aspect, the present disclosure provides a method of treating or alleviating a symptom of cancer by administering to a subject in need thereof a therapeutically effective amount of a DNMT inhibitor, prior to administering a therapeutically effective amount of Compound A2 or a pharmaceutically acceptable salt, polymorph, solvate, or stereoisomer thereof.

In one aspect, the present disclosure provides a method of treating or alleviating a symptom of cancer by administering to a subject in need thereof a therapeutically effective amount of a DNMT inhibitor, wherein the therapeutically effective amount is an amount sufficient to sensitize the subject to a subsequent treatment with Compound A2 or a pharmaceutically acceptable salt, polymorph, solvate, or stereoisomer thereof. The method may further include a step of administering to the sensitized subject a therapeutically effective amount of Compound A2 or a pharmaceutically acceptable salt, polymorph, solvate, or stereoisomer thereof.

In one aspect, the present invention provides a method of treating or alleviating a symptom of cancer by administering to a subject in need thereof a therapeutically effective amount of a DNMT inhibitor, prior to administering a therapeutically effective amount of a combination of a DNMT inhibitor and Compound A2 or a pharmaceutically acceptable salt, polymorph, solvate, or stereoisomer thereof.

In one aspect, the present invention provides a method of treating or alleviating a symptom of cancer by administering to a subject in need thereof a therapeutically effective amount of a DNMT inhibitor, wherein the therapeutically effective amount is an amount sufficient to sensitize the subject to a subsequent treatment with a combination of a DNMT inhibitor and Compound A2 or a pharmaceutically acceptable salt, polymorph, solvate, or stereoisomer thereof. The method may further include a step of administering to the sensitized subject a therapeutically effective amount of a combination of a DNMT inhibitor and Compound A2 or a pharmaceutically acceptable salt, polymorph, solvate, or stereoisomer thereof.

In one aspect, multiple doses of DNMT inhibitor are administered prior to the administration of Compound A2.

In one aspect, multiple doses of DNMT inhibitor are administered prior to the administration of the combination of DNMT inhibitor and Compound A2.

In certain embodiments, the DNMT inhibitor is administered continuously without a drug holiday prior to administration of Compound A2. For example, the DNMT inhibitor is administered at a single dose per day for, e.g., 2, 3, 4, or 5 days before administration of Compound A2. In certain embodiments, the DNMT inhibitor is administered with a drug holiday prior to administration of Compound A2. For example, the DNMT inhibitor is administered at a single dose per day at day 1 and Compound A2 is administered after, e.g., at least 2, 3, 4, 5, 6, or 7 days after administration of the DNMT inhibitor.

In certain embodiments, Compound A2 or a pharmaceutically acceptable salt, polymorph, solvate, or stereoisomer thereof is administered at least one, two, three or more hours following the administration of the DNMT inhibitor.

In certain embodiments, Compound A2 or a pharmaceutically acceptable salt, polymorph, solvate, or stereoisomer thereof is administered at least one, two, three or more days following the administration of the DNMT inhibitor.

In certain embodiments, a first DNMT inhibitor is administered continuously without a drug holiday prior to administration of the combination of Compound A2 and a second DNMT inhibitor (which may be the same as or different from the first DNMT inhibitor). For example, the first DNMT inhibitor is administered at a single dose per day for, e.g., 2, 3, 4, or 5 days before administration of the combination. In certain embodiments, a first DNMT inhibitor is administered with a drug holiday prior to administration of the combination of Compound A2 and a second DNMT inhibitor (which may be the same as or different from the first DNMT inhibitor). For example, the first DNMT inhibitor is administered at a single dose per day at day 1 and the combination is administered after, e.g., at least 2, 3, 4, 5, 6, or 7 days after administration of the first DNMT inhibitor.

In certain embodiments, the combination of the second DNMT inhibitor and Compound A2 or a pharmaceutically acceptable salt, polymorph, solvate, or stereoisomer thereof is administered at least one, two, three or more hours following the administration of the first DNMT inhibitor.

In certain embodiments, the combination of the second DNMT inhibitor and Compound A2 or a pharmaceutically acceptable salt, polymorph, solvate, or stereoisomer thereof is administered at least one, two, three or more days following the administration of the first DNMT inhibitor.

In certain embodiments, the sensitization is determined by the methylation status of histones or other proteins.

In certain embodiments, the sensitization is determined by a decreased level of methylation of histones of other proteins as compared to a subject that was not sensitized with a DNMT inhibitor.

In certain embodiments, the sensitization is determined by decreased methylation of H3K79.

In certain embodiments, the therapeutically effective amount of Compound A2 or a pharmaceutically acceptable salt, polymorph, solvate, or stereoisomer thereof is lowered due to the sensitizing effect of the DNMT inhibitor.

In certain embodiments, the therapeutically effective amount of the combination of a DNMT inhibitor and Compound A2 or a pharmaceutically acceptable salt, polymorph, solvate, or stereoisomer thereof is lowered due to the sensitizing effect of the DNMT inhibitor.

In some embodiments, the combination of a DNMT inhibitor and Compound A2 or a pharmaceutically acceptable salt, polymorph, solvate, or stereoisomer thereof described herein is administered to the subject in need thereof at a dosage of 0.01 mg/kg per day to about 1000 mg/kg per day.

In some embodiments, Compound A2 (i.e., EPZ-5676) or a pharmaceutically acceptable salt, polymorph, solvate, or stereoisomer thereof is administered at a dosage of 0.01 mg/kg per day to about 1000 mg/kg per day.

In some embodiments, the DNMT inhibitor is administered at a dosage of 0.01 mg/kg per day to about 1000 mg/kg per day.

In some embodiments, the DNMT inhibitor is Azacitidine. For example, Azacitidine is administered at a dosage of 100 mg/m²/day or less (e.g., 75 mg/m²/day or less, 50 mg/m²/day or less, 25 mg/m²/day or less, 15 mg/m²/day or less, 10 mg/m²/day or less, or 5 mg/m²/day or less, or 1-100 mg/m²/day, 5-75 mg/m²/day, 1-5 mg/m²/day, 1-10 mg/m²/day, 1-15 mg/m²/day, 1-25 mg/m²/day, 1-50 mg/m²/day, or 1-75 mg/m²/day).

In some embodiments, EPZ-5676 or a pharmaceutically acceptable salt, polymorph, solvate, or stereoisomer thereof is administered at a dose of at least 12, 24, 36, 45, 54, 70, 80, or 90 mg/m²/day.

In some embodiments, EPZ-5676 or a pharmaceutically acceptable salt, polymorph, solvate, or stereoisomer thereof is administered continuously for at least 2, 3, 4, 5, 6, 7, 14, 21, 28, 35, 42, 47, 56, or 64 days.

In some embodiments, the DNMT inhibitor is administered continuously for at least 2, 3, 4, 5, 6, 7, 14, 21, 28, 35, 42, 47, 56, or 64 days.

In some embodiments, continuous administration comprises administration without a drug holiday (e.g., EPZ-5676 or its N-oxide or a pharmaceutically acceptable salt thereof is administered continuously, e.g., via intravenous (“IV”) infusion, for 28 days in a 28-day cycle).

In some embodiments, the compound disclosed herein (e.g., the DNMT inhibitor or EPZ-5676) is administered with a drug holiday. For example, EPZ-5676 or its N-oxide or a pharmaceutically acceptable salt thereof is administered, e.g., via IV infusion, for 21 days of a 28-day cycle with a 7-day drug holiday per cycle.

In some embodiments, the administration results in maturation or differentiation of leukemic blast cells. For example, at least 20% of leukemic blast cells have undergone maturation or differentiation. For example, at least 50% of leukemic blast cells have undergone maturation or differentiation. For example, at least 80% of leukemic blast cells have undergone maturation or differentiation.

In some embodiments, administration results in reduction of H3K79 methyl mark to at least 90%, 80%, 70%, 60%, 50%, 40%, 30%, 20%, 10% or less of untreated control levels.

In some embodiments, administration results in the suppression of H3K79 methyl mark rebound.

In some embodiments, administration results in at least 20%, 30%, 40%, 50%, 60%, 70%, 80%, or 90% of leukemic blast cells undergoing cell death or apoptosis.

In some embodiments, the method of treatment includes resolution of fevers, resolution of cachexia or resolution of leukemia cutis.

In some embodiments, the method of treatment includes restoration of normal haematopoiesis.

In some embodiments, the subject has demonstrated resistance to any one of the components of a combination described herein when administered as a single agent.

In some embodiments, the subject is a pediatric patient aged 3 months to 18 years.

In one aspect, the present disclosure provides a method of inhibiting cancer cell proliferation by contacting a cancer cell with a composition described herein.

In one aspect, the present disclosure provides a method of inhibiting cancer cell proliferation by contacting a cancer cell with an effective amount of a DNMT inhibitor, prior to administering an effective amount of Compound A2 or a salt, polymorph, solvate, or stereoisomer thereof, or prior to administering an effective amount of a combination of a DNMT inhibitor and Compound A2 or a salt, polymorph, solvate, or stereoisomer thereof. For example, the effective amount of the DNMT inhibitor is at least 0.01 μM (e.g., at least 0.1 μM, at least 0.3 μM, at least 0.5 μM or at least 1 μM).

The subject may have leukemia. The leukemia may be characterized by a chromosomal rearrangement. The chromosomal rearrangement is chimeric fusion of mixed lineage leukemia gene (MLL) or partial tandem duplication of MLL (MLL-PTD).

The subject may have an increased level of HOXA9, Fms-like tyrosine kinase 3 (FLT3), MEIS1, MEIS2, TBP, BCL, and/or DOT1L.

Unless otherwise defined, all technical and scientific terms used herein have the same meaning as commonly understood by one of ordinary skill in the art to which this invention belongs. In the specification, the singular forms also include the plural unless the context clearly dictates otherwise. Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Any of the above aspects and embodiments can be combined with any other aspect or embodiment.

Other features and advantages of the invention will be apparent from the following detailed description and claims.

### BRIEF DESCRIPTION OF DRAWINGS

The above and further features will be more clearly appreciated from the following detailed description when taken in conjunction with the accompanying drawings.

FIGS. 1A-1D are a series of graphs that depict mRNA expression levels of MEIS1 (FIGS. 1A and 1C) and HOXA2 (FIGS. 1B and 1D) in the MV4-11 cell line following treatment with AZA (also referred to herein as Azacitidine) and EPZ-5676. The data depicted in FIGS. 1A and 1B are from assays in which various concentrations of EPZ-5676 or DMSO alone, in combination with 1 μM AZA QD×3, 0.3 μM AZA QD×3, or vehicle alone were contacted with the cultured MV4-11 cells. (QD=daily dosage; QD×3=three day treatment). The data indicate a decrease in the amounts of MEIS1 and HOXA2 mRNA levels following contacting the cells with increased concentrations of AZA and EPZ 5676. The data depicted in FIGS. 1C and 1D demonstrate that AZA priming leads to rapid depletion of MEIS1 RNA levels within 2 days of EPZ-5676 treatment. The data also show that AZA priming followed by EPZ-5676 exposure leads to synergy in differentiation (as shown by CD14+ population).

FIGS. 2A and 2B demonstrate that AZA priming followed by EPZ-5676 leads to enhanced cell death. FIG. 2A is a schematic that depicts the experimental setup for the data presented in FIG. 2B. FIG. 2A depicts three experimental conditions: 1) MV4-11 cells were contacted with AZA for three consecutive days, followed by treatment with EPZ-5676 on the second, fourth, sixth, and ninth day; 2) the MV4-11 cells were contacted with vehicle for three consecutive days, followed by treatment with EPZ-5676 on the second, fourth, sixth, and ninth day; and 3) the MV4-11 cells were contacted with vehicle for three consecutive days, followed by contact with vehicle on the second, fourth, sixth, and ninth day. FIG. 2B is a graph that demonstrates that AZA priming followed by EPZ-5676 treatment leads to enhanced cell death.

FIGS. 3A-3D are a series of graphs that contain data obtained from H3K79me2 ChIP-qPCR assays of MV4-11 (MLL-AF4) cells at various time points following exposure to either vehicle alone, vehicle and 0.5 μM EPZ-5676, or 0.5 μM QD×3 AZA and 0.5 μM EPZ-5676. The data indicate that H3K79me2 signal is reduced to comparable levels with EPZ-5676 treatment, with and without AZA priming.

FIGS. 4A-4H are a series of schematics and graphs that depict the effect on cellular differentiation of AZA priming in AML cell lines. FIGS. 4A and 4B are a series of schematics that depict the experimental setup of the assays performed to determine differentiation and cell death. FIG. 4C depicts AZA dose response curves for an AML cell line panel. FIGS. 4D-4G are a series of flow cytometry assays that were performed to assess cellular differentiation and cell death in the NOMO-1 acute myeloid leukemia cell line. For the flow cytometry assays, Annexin V/7-AAD staining was used to assess cell death, and CD11b/CD14 antibodies were used to evaluate cellular differentiation. The flow cytometry data indicate that there is no appreciable cell death following treatment of the cells with either vehicle or 0.5 μM EPZ-5676 (FIGS. 4D and 4E). The flow cytometry data indicate that there is an increase in the amount of CD14+ cells following contact with 0.5 μM EPZ-5676 (FIGS. 4D and 4E). Flow cytometry assays were also performed to assess cellular differentiation and cell death following AZA priming followed by treatment of the cells with 0.5 μM EPZ-5676. The data from these assays indicate that AZA priming, followed by treatment with 0.5 μM EPZ-5676 does not increase cell death, while there is an increase in the amounts of CD14+ cells in the population (FIGS. 4F and 4G). FIG. 4H is a graph that depicts a synergistic increase of CD14+ population upon AZA priming by day 6.

FIGS. 5A-5F are a series of graphs that depict real-time imaging to study the effect of AZA priming on MV4-11 cells utilizing Incucyte Zoom® for suspension cell imaging. FIG. 5A shows a comparison of cellular growth between MV4-11 cells containing a lentivirus mCherry reporter and MV4-11 cells that are unmodified (Parental). The Kinetic Caspase 3/7 reagent was used to assess cell death (FIG. 5B). For these assays, 6,000 cells were plated per well of a 96-well plate and dosed with EPZ-5676 using a ten point concentration gradient starting from 10 μM. 1× caspase dye was then added to each well. FIG. 5C is a series of graphs that depict real-time monitoring of cell death using Caspase 3/7 kinetic dye. FIG. 5D is a series of graphs that depict rapid cell killing following exposure to either vehicle or AZA priming followed by treatment with EPZ-5676, and control conditions including vehicle alone, DMSO. FIG. 5E is a series of graphs that depict real-time monitoring of AUC Growth by Red object (labeled cell) count. FIG. 5F is a series of graphs that depicts cellular AUC growth following vehicle or AZA priming followed by treatment with EPZ-5676, and control conditions including vehicle alone, DMSO.

FIGS. 6A-6D are a series of flow cytometry graphs that depict cellular differentiation and cell death following treatment of MV4-11 (MLL-AF4) cells with vehicle or AZA priming followed by 0.5 μM EPZ-5676, or vehicle only conditions. The data indicate that there is no appreciable cell death by treatment with EPZ-5676 alone, and that there is no increase in cellular differentiation as indicated by no increase in the CD14 positive population (FIGS. 6A and 6B). The data also indicate that there is increased cell death upon AZA priming, while there is no increase in the CD14 positive population (FIGS. 6C and 6D).

FIGS. 7A-7D are a series of flow cytometry graphs that depict cellular differentiation and cell death following treatment of RS4;11 (MLL-AF4) cells with vehicle or AZA priming followed by 0.5 μM EPZ-5676, or vehicle only conditions. The data indicate no cell death and no cell differentiation with vehicle or EPZ-5676 treatment alone (FIGS. 7A and 7B). The data also indicate that AZA had an effect on cell death, but had no effect on cellular differentiation (FIGS. 7C and 7D).

FIGS. 8A-8D are a series of flow cytometry graphs that depict cellular differentiation and cell death following treatment of ML-2 (MLL-AF6) cells with vehicle or AZA priming following by 0.5 μM EPZ-5676, or vehicle only conditions. The data indicate no cell death and no cell differentiation with vehicle or EPZ-5676 treatment alone (FIGS. 8A and 8B). The data also indicate that AZA had an effect on cell death, but had no effect on cellular differentiation (FIGS. 8C and 8D).

FIGS. 9A and 9B are a series of schematics depicting the AZA and EPZ-5676 treatment of cells. FIGS. 9A and 9B also depict the results of the CellTiter-Glo® Luminescent Cell Viability Assay (CTG) of MV4-11 cells.

FIGS. 10A-10C are a series of schematics and graphs that depict AZA and EPZ-5676 treatment of MV4-11 cells and the treatment's effect on cell death.

FIGS. 11A and 11B are a series of schematics and graphs that depict AZA and EPZ-5676 treatment of THP-1 cells and the treatment's effect on cell death.

FIG. 11C depicts a graph and table that depict the viability of cells following a QD×3 AZA treatment regimen.

FIGS. 12A and 12B are a series of graphs that depict AZA priming followed by EPZ-5676 treatment of MV4-11 (MLL-AF4) cells and the effects of said treatment on cellular viability.

FIGS. 13A and 13B are a series of graphs that depict AZA priming followed by EPZ-5676 treatment of SEM (MLL-AF4) cells and the effects of said treatment on cellular viability.

FIGS. 14A and 14B are a series of graphs that depict AZA priming followed by EPZ-5676 treatment of MOLM-13 (MLL-AF9) cells and the effects of said treatment on cellular viability.

FIGS. 15A and 15B are a series of graphs that depict AZA priming followed by EPZ-5676 treatment of RS4;11(MLL-AF4) cells and the effects of said treatment on cellular viability.

FIGS. 16A and 16B are a series of graphs that depict AZA priming followed by EPZ-5676 treatment of ML-2(MLL-AF6) cells and the effects of said treatment on cellular viability.

FIG. 17 is a plot of fraction affected versus log concentration of Azacitidine, which demonstrates that a combination benefit is found with MOLM-13 cells treated with Azacitidine 7 days prior to a 3 day co-treatment with Azacitidine and EPZ-5676.

FIG. 18 is a table demonstrating that synergistic cell killing with EPZ-5676 is achieved in MLL-rearranged leukemia cell lines sensitive to both EPZ-5676 and Azacitidine.

FIG. 19 comprises plots (A) and (B) which demonstrate results from an MV4-11 efficacy study with EPZ-5676 treated in combination with Azacitidine or Cytarabine. (A) is a plot of median tumor volume versus day and (B) is a plot of percent body weight versus day.

### DETAILED DESCRIPTION

The present disclosure is based upon the discovery that DOT1L histone methyltransferase inhibitors and anti-cancer agents, especially DNMT inhibitor anti-cancer agent, can be used in combination to treat tumors and with superior results than those achieved by treating tumors with DOT1L histone methyltransferase inhibitors alone or anti-cancer agents alone.

Accordingly, the present disclosure provides a combination comprising a DOT1L histone methyltransferase inhibitor and one or more DNMT inhibitors, and methods for their use to treat diseases the course of which can be influenced by modulating the methylation status of histones or other proteins, e.g., cancer. In particular, the present disclosure features a combination comprising Compound A2 and Azacitidine.

The present disclosure also includes methods for combination therapies comprising DOT1L histone methyltransferase inhibitor and one or more DNMT inhibitors, such as Compound A2 and Azacitidine to treat cancer, e.g., leukemia. Specifically, the methods of the present disclosure are useful for treating or inhibiting cancer cell proliferation.

The present disclosure further provides uses of any compositions or combinations described herein in the manufacture of medicament for treating diseases. Such diseases include, for example, cancer, a precancerous condition, or a disease influenced by modulating the methylation status of histones or other proteins.

Any compound (e.g., a DOT1L inhibitor or a DNMT inhibitor) disclosed herein can be used for the compositions or combination therapy of the disclosure. As used herein, a DOT1L inhibitor is an inhibitor of DOT1L-mediated protein methylation (e.g., an inhibitor of histone methylation). In some embodiments, a DOT1L inhibitor is a small molecule inhibitor of DOT1L.

In one aspect, a composition of the disclosure comprises EPZ-5676 or a pharmaceutically acceptable salt, polymorph, solvate, or stereoisomer thereof, and one or more DNMT inhibitors. EPZ-5676 is suitable for administration as part of a combination therapy with one or more DNMT inhibitors (such as Azacitidine) or treatment modality, suitable to be administered together, sequentially, or in alternation.

Other DOT1L inhibitors suitable for use according to methods described herein are provided in WO2012/075381, WO2012/075492, WO2012/082436, WO2012/75500, WO2014/026198, WO2014/035140, WO2014/153001, US2014/0100184, and in J. Med Chem. (2013), 56: p. 8972-8983, the contents of each of which are hereby incorporated by reference in their entireties.

The disclosure also relates to a combination of a pharmaceutical composition comprising a therapeutically effective amount of a DNMT inhibitor and a pharmaceutically acceptable carrier and a pharmaceutical composition comprising a therapeutically effective amount of a DOT1L inhibitor and a pharmaceutically acceptable carrier.

The disclosure also relates to a combination of a pharmaceutical composition comprising a therapeutically effective amount of a DNMT inhibitor and a pharmaceutically acceptable carrier and a pharmaceutical composition comprising a therapeutically effective amount of Compound A2 or a salt, hydrate, polymorph, solvate, stereoisomer thereof and a pharmaceutically acceptable carrier.

In addition, the disclosure provides methods of synthesizing the foregoing compounds. Following synthesis, a therapeutically effective amount of one or more of the compounds can be formulated with a pharmaceutically acceptable carrier for administration to a mammal, particularly humans, for use in modulating an epigenetic enzyme. In certain embodiments, the compounds disclosed herein are useful for treating, preventing, or reducing the risk of cancer or for the manufacture of a medicament for treating, preventing, or reducing the risk of cancer. Accordingly, the compounds, compositions, or the formulations can be administered, for example, via oral, parenteral, otic, ophthalmic, nasal, or topical routes, to provide an effective amount of the compound to the mammal.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds disclosed herein may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms.

Furthermore, the structures and other compounds disclosed herein include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. Benzimidazoles also exhibit tautomerism, when the benzimidazole contains one or more substituents in the 4, 5, 6 or 7 positions, the possibility of different isomers arises. For example, 2,5-dimethyl-1H-benzo[d]imidazole can exist in equilibrium with its isomer 2,6-dimethyl-1H-benzo[d]imidazole via tautomerization.

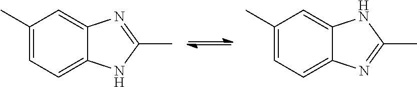

### 2,5-dimethyl-1H-benzo[d]imidazole 2,6-dimethyl-1H-benzo[d]imidazole

Another example of tautomerism is shown below.

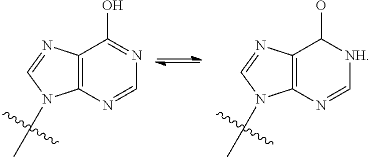 [SMILES: *n1cnc2c(O)ncnc21 ; *n1cnc2c1N=CNC2O]

It is to be understood that the compounds disclosed herein may be depicted as different tautomers. It should also be understood that when compounds have tautomeric forms, all tautomeric forms are intended to be included in the scope of the present disclosure, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectra, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Compounds disclosed herein may be crystalline, semi-crystalline, non-crystalline, amorphous, and mesomorphous.

The compounds disclosed herein include the compounds themselves, as well as their N-oxides, salts, their solvates, their polymorphs, and their stereoisomers, if applicable. A salt, for example, can be formed between an anion and a positively charged group (e.g., amino) on the compound or inhibitor (e.g., a substituted nucleoside compound such as a substituted purine or 7-deazapurine compound). Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate. Likewise, a salt can also be formed between a cation and a negatively charged group (e.g., carboxylate) on the compound or inhibitor (e.g., a substituted nucleoside compound such as a substituted purine or 7-deazapurine compound). Suitable cations include sodium ion, potassium ion, magnesium ion, calcium ion, and an ammonium cation such as tetramethylammonium ion. The compound or inhibitor (e.g., a substituted nucleoside compound such as a substituted purine or 7-deazapurine compound) also include those salts containing quaternary nitrogen atoms.

Additionally, the compounds disclosed herein, for example, the salts of the compounds, can exist in either hydrated or unhydrated (the anhydrous) form or as solvates with other solvent molecules. Nonlimiting examples of hydrates include hemihydrates, monohydrates, dihydrates, trihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hemihydrate is formed by the combination of one molecule of water with more than one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not necessarily in structure or origin to the reference compound.

As defined herein, the term “derivative” refers to compounds that have a common core structure, and are substituted with various groups as described herein. For example, Compound A2 and its derivatives are substituted purine compounds or substituted 7-deazapurine compounds, and have purine or 7-deazapurine as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The present invention also provides methods for the synthesis of the compounds of any of the Formulae disclosed herein. The present invention also provides detailed methods for the synthesis of various disclosed compounds according to the schemes and the Examples described in WO2012/075381, WO2012/075492, WO2012/082436, WO2012/75500, WO2014/026198, WO2014/035140, US2014/0100184, and in J. Med Chem. (2013), 56: p. 8972-8983, the contents of which are hereby incorporated by reference in their entireties.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial unless otherwise specified so long as the invention remains operable. Moreover, two or more steps or actions can be conducted simultaneously.

Throughout the description, where compositions are described as having, including, or comprising specific components, or where processes are described as having, including, or comprising specific process steps, it is contemplated that compositions or combinations of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the invention remains operable. Moreover, two or more steps or actions can be conducted simultaneously.

Compounds suitable for the methods of the disclosure, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described herein.

To further assess a compound's drug-like properties, measurements of inhibition of cytochrome P450 enzymes and phase II metabolizing enzyme activity can also be measured either using recombinant human enzyme systems or more complex systems like human liver microsomes. Further, compounds can be assessed as substrates of these metabolic enzyme activities as well. These activities are useful in determining the potential of a compound to cause drug-drug interactions or generate metabolites that retain or have no useful antimicrobial activity.

To get an estimate of the potential of the compound to be orally bioavailable, one can also perform solubility and Caco-2 assays. The latter is a cell line from human epithelium that allows measurement of drug uptake and passage through a Caco-2 cell monolayer often growing within wells of a 24-well microtiter plate equipped with a 1 micron membrane. Free drug concentrations can be measured on the basolateral side of the monolayer, assessing the amount of drug that can pass through the intestinal monolayer. Appropriate controls to ensure monolayer integrity and tightness of gap junctions are needed. Using this same system one can get an estimate of P-glycoprotein mediated efflux. P-glycoprotein is a pump that localizes to the apical membrane of cells, forming polarized monolayers. This pump can abrogate the active or passive uptake across the Caco-2 cell membrane, resulting in less drug passing through the intestinal epithelial layer. These results are often done in conjunction with solubility measurements and both of these factors are known to contribute to oral bioavailability in mammals. Measurements of oral bioavailability in animals and ultimately in man using traditional pharmacokinetic experiments will determine the absolute oral bioavailability.

Experimental results can also be used to build models that help predict physical-chemical parameters that contribute to drug-like properties. When such a model is verified, experimental methodology can be reduced, with increased reliance on the model predictability.

The present disclosure provides methods for combination therapy in which EPZ-5676 or a pharmaceutically acceptable salt, polymorph, solvate, or stereoisomer thereof, and a DNMT inhibitor are administered simultaneously, sequentially, or in alternation to a subject in need for treatment of a disease or cancer. For example, EPZ-5676 or a pharmaceutically acceptable salt, polymorph, solvate, or stereoisomer thereof, and one or more DNMT inhibitors can be administered as a co-formulation or separate formulations, wherein the administration of formulations is simultaneous, sequential, or in alternation. The combination therapy can also be administered to cancer cells to inhibit proliferation or induce cell death.

In one aspect, a DNMT inhibitor is administered to a subject in need thereof prior to administration of EPZ-5676 or a pharmaceutically acceptable salt, polymorph, solvate, or stereoisomer thereof.

In one aspect, a DNMT inhibitor is administered prior to administration of the combination of the disclosure comprising EPZ-5676 or a pharmaceutically acceptable salt, polymorph, solvate, or stereoisomer thereof, and one or more DNMT inhibitors.

In one embodiment, the one or more DNMT inhibitors can be an agent that is recognized in the art as being useful to treat the disease or condition being treated by the composition or combination therapy of the present disclosure. In another embodiment, the one or more DNMT inhibitors can be an agent that is not recognized in the art as being useful to treat the disease or condition being treated by the composition or combination therapy of the present disclosure. In one aspect, the DNMT inhibitor can be an agent that imparts a beneficial attribute to EPZ-5676 or the composition or combination therapy of the present disclosure (e.g., resulting a synergistic effect). The beneficial attribute to the composition or combination therapy of the present disclosure includes, but is not limited to, pharmacokinetic or pharmacodynamic co-action resulting from the combination of EPZ-5676 and one or more DNMT inhibitors.

In some embodiments, the DNMT inhibitor is an anticancer agent.

In some embodiments, the DNMT inhibitor is a nucleoside analog, e.g., a cytidine analog such as Azacitidine, Decitabine, and Zebularine, or functional analogs, derivatives, prodrugs, and metabolites thereof.

In some embodiments, the DNMT inhibitor is a non-nucleoside DNMT inhibitor such as RG108, Psammaplin A, Procainamide hydrochloride or functional analogs, derivatives, prodrugs, and metabolites thereof.

In some embodiments, the DNMT inhibitor is selected from those disclosed in Gros et al., “DNA methylation inhibitors in cancer: Recent and future approaches” Biochimie. 2012; 94(11):2280-96, the contents of which are hereby incorporated by reference in its entirety.

In some embodiments, the DNMT inhibitor shows a synergistic effect in combination with Compound A2 in Molm13 and/or MV4-11 cells.

The DNMT inhibitors disclosed herewith are for illustrative purposes and not intended to be limiting. The present disclosure can include more than one DNMT inhibitor, e.g., two, three, four, or five DNMT inhibitors such that the combination therapy of the present invention can perform its intended function.

In some embodiments, the one or more DNMT inhibitors are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more prior to the administration of EPZ-5676 or a pharmaceutically acceptable salt, polymorph, solvate, or stereoisomer thereof.

In one aspect, multiple doses of a DNMT inhibitor are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more prior to the administration of EPZ-5676 or a pharmaceutically acceptable salt, polymorph, solvate, or stereoisomer thereof.

In some embodiments, the one or more DNMT inhibitors are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more prior to the administration of a combination of EPZ-5676 or a pharmaceutically acceptable salt, polymorph, solvate, or stereoisomer thereof and a second DNMT inhibitor (which may be the same as or different from the one or more DNMT inhibitors).

In one aspect, multiple doses of a DNMT inhibitor are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more prior to the administration of a combination of EPZ-5676 or a pharmaceutically acceptable salt, polymorph, solvate, or stereoisomer thereof and a second DNMT inhibitor (which may be the same as or different from the multiple doses of the DNMT inhibitors).

In some embodiments, the one or more DNMT inhibitors (either in a single dose or multiple doses) are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more prior to the administration of EPZ-5676 or a pharmaceutically acceptable salt, polymorph, solvate, or stereoisomer thereof. In some embodiments, the one or more DNMT inhibitors (either in a single dose or multiple doses) are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more prior to the administration of a combination of EPZ-5676 or a pharmaceutically acceptable salt, polymorph, solvate, or stereoisomer thereof and a second DNMT inhibitor (which may be the same as or different from the one or more DNMT inhibitors).

It should be appreciated that Compound A2 or the combination comprising Compound A2 or a pharmaceutically acceptable salt, polymorph, solvate, or stereoisomer thereof and a second DNMT inhibitor, can be administered to a subject after the level in a subject of the first DNMT inhibitor that has been administered to the subject has decreased. Thus, for instance, a first DNMT inhibitor is administered to a subject and Compound A2 or a combination of Compound A2 and a second DNMT inhibitor are administered after the level of administered first DNMT inhibitor is less than 90% of the initial level, less than 80% of the initial level, less than 70% of the initial level, less than 60% of the initial level, less than 50% of the initial level, less than 40% of the initial level, less than 30% of the initial level, less than 20% of the initial level or less than 10% of the initial level. In some embodiments, the first DNMT inhibitor that has been administered to a subject can no longer be detected in a subject prior to administration of Compound A2 or a combination of Compound A2 and a second DNMT inhibitor.

In one aspect, the disclosure provides methods for sensitizing or priming a subject to administration of Compound A2 or a pharmaceutically acceptable salt, polymorph, solvate, or stereoisomer thereof. In some embodiments, a subject is sensitized or primed for responding to Compound A2 or a pharmaceutically acceptable salt, polymorph, solvate, or stereoisomer thereof by administering one or more DNMT inhibitors (e.g., anti-cancer agents). Thus, in one aspect, a first DNMT inhibitor is administered to a subject prior to the administration of Compound A2 or a pharmaceutically acceptable salt, polymorph, solvate, or stereoisomer thereof or a combination comprising a second DNMT inhibitor and Compound A2 or a pharmaceutically acceptable salt, polymorph, solvate, or stereoisomer thereof, resulting in the sensitization or priming of the subject. Consequently the subject is more sensitive to Compound A2 or a pharmaceutically acceptable salt, polymorph, solvate, or stereoisomer thereof. The second DNMT inhibitor may be the same as or different from the first DNMT inhibitor.

In some embodiments, the administration of a first DNMT inhibitor (e.g., Azacitidine) results in a biological effect prior to the administration of Compound A2 or a pharmaceutically acceptable salt, polymorph, solvate, or stereoisomer thereof or the combination comprising and Compound A2 or a pharmaceutically acceptable salt, polymorph, solvate, or stereoisomer thereof and a second DNMT inhibitor (e.g., Azacitidine). In some embodiments, Compound A2 or a pharmaceutically acceptable salt, polymorph, solvate, or stereoisomer thereof is not administered until 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of the first DNMT inhibitor has resulted in a biological effect. In some embodiments, the biological effect is a reduction of H3K79 methyl mark, maturation or induction of blast cells, apoptosis of leukemic blast cells, resolution of fevers, cachexia or leukemia cutis and/or restoration of normal haemoatopoiesis. It should be appreciated that more than one biological effect may result from the administration of the first DNMT inhibitor. In some embodiments, the biological effect is a reduction of H3K79 methyl mark. In some embodiments, the biological effect is a reduction of H3K79 methyl mark to at least 90%, 80%, 70%, 60%, 50%, 40%, 30%, 20%, 10% or less compared to untreated control levels. In some embodiments, the H3K79 methyl mark must be at least 90%, 80%, 70%, 60%, 50%, 40%, 30%, 20%, 10% or less compared to untreated control levels prior to the addition of Compound A2 or a pharmaceutically acceptable salt, polymorph, solvate, or stereoisomer thereof.

In some embodiments, the biological effect is the maturation or differentiation of leukemic blast cells. In some embodiments, at least 20% of leukemic blast cells have undergone maturation or differentiation, at least 50% of leukemic blast cells have undergone maturation or differentiation, or at least 80% of leukemic blast cells have undergone maturation or differentiation prior to the addition of Compound A2 or a pharmaceutically acceptable salt, polymorph, solvate, or stereoisomer thereof.

In some embodiments, the biological effect is the apoptosis of leukemic blast cells. In some embodiments, at least 20%, 30%, 40%, 50%, 60%, 70%, 80%, or 90% of the leukemic blast cells undergo cell death or apoptosis prior to administration of Compound A2 or a pharmaceutically acceptable salt, polymorph, solvate, or stereoisomer thereof. In some embodiments, the biological effect is the resolution of fever, resolution of cachexia and/or resolution of leukemia cutis. In some embodiments, fever, cachexia and/or leukemia cutis is resolved prior to administration of Compound A2 or a pharmaceutically acceptable salt, polymorph, solvate, or stereoisomer thereof. In some embodiments, the biological effect is the restoration of normal haematopoiesis. In some embodiments, normal haematopoiesis is restored prior to administration of Compound A2 or a pharmaceutically acceptable salt, polymorph, solvate, or stereoisomer thereof.

In some embodiments, a subject is evaluated after the administration of a first DNMT inhibitor (e.g., Azacitidine) for any biological effects prior to administration of Compound A2 or a pharmaceutically acceptable salt, polymorph, solvate, or stereoisomer thereof or the combination comprising Compound A2 or a pharmaceutically acceptable salt, polymorph, solvate, or stereoisomer thereof and a second DNMT inhibitor (e.g., Azacitidine). In some embodiments, Compound A2 or a pharmaceutically acceptable salt, polymorph, solvate, or stereoisomer thereof is administered only if the evaluated biological effect has reached a certain predetermined level or activity. In some embodiments, the biological effect is maturation or induction of blast cells, apoptosis of leukemic blast cells, resolution of fever, cachexia or leukemia cutis and/or restoration of normal haemoatopoiesis. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of the first DNMT inhibitor.

In certain aspects of the disclosure, the sensitization or priming by a first DNMT inhibitor results in the need for lower therapeutically effective amounts of the sequential administration of Compound A2 or a pharmaceutically acceptable salt, polymorph, solvate, or stereoisomer thereof or a combination of the disclosure comprising Compound A2 or a pharmaceutically acceptable salt, polymorph, solvate, or stereoisomer thereof and a second DNMT inhibitor. It should be appreciated that in certain embodiments, the sensitization would result in a synergistic effect as described herein between Compound A2 or a pharmaceutically acceptable salt, polymorph, solvate, or stereoisomer thereof and the DNMT inhibitors, such as Azacitidine, Decitabine, or Zebularine.

In one aspect, Compound A2 or a pharmaceutically acceptable salt, polymorph, solvate, or stereoisomer thereof is administered continuously. In some embodiments, Compound A2 or a pharmaceutically acceptable salt, polymorph, solvate, or stereoisomer thereof is administered continuously for at least 7, 14, 21, 28, 35, 42, 47, 56 or 64 days. In some embodiments, Compound A2 or a pharmaceutically acceptable salt, polymorph, solvate, or stereoisomer thereof is administered without a drug holiday.

In one aspect, Compound A2 or a pharmaceutically acceptable salt, polymorph, solvate, or stereoisomer thereof and one or more DNMT inhibitors are administered simultaneously or sequentially. In some embodiments, Compound A2 or a pharmaceutically acceptable salt, polymorph, solvate, or stereoisomer thereof and the one or more DNMT inhibitors are administered continuously. In some embodiments, Compound A2 or a pharmaceutically acceptable salt, polymorph, solvate, or stereoisomer thereof and the one or more DNMT inhibitors are administered continuously for at least 7, 14, 21, 28, 35, 42, 47, 56 or 64 days. In some embodiments, Compound A2 or a pharmaceutically acceptable salt, polymorph, solvate, or stereoisomer thereof and the one or more DNMT inhibitors are administered without a drug holiday.

In one aspect, Compound A2 or a pharmaceutically acceptable salt, polymorph, solvate, or stereoisomer thereof is administered continuously while the one or more DNMT inhibitors are not administered continuously. In some embodiments, Compound A2 or a pharmaceutically acceptable salt, polymorph, solvate, or stereoisomer thereof is administered continuously for at least 7, 14, 21, 28, 35, 42, 47, 56 or 64 days while the one or more DNMT inhibitors are not administered continuously for at least 7, 14, 21, 28, 35, 42, 47, 56 or 64 days. In some embodiments, Compound A2 or a pharmaceutically acceptable salt, polymorph, solvate, or stereoisomer thereof is administered without a drug holiday while the one or more DNMT inhibitors are administered with a drug holiday. It should be appreciated that Compound A2 or a pharmaceutically acceptable salt, polymorph, solvate, or stereoisomer thereof and the one or more DNMT inhibitors can be administered using different regimens. Thus, for instance, Compound A2 or a pharmaceutically acceptable salt, polymorph, solvate, or stereoisomer thereof may be administered continuously while the one or more DNMT inhibitors may be administered as one dose or a defined number of multiple doses. The administration regimen of the one or more DNMT inhibitors may be as indicated on a label (e.g., if the DNMT inhibitor is a regulated drug) and/or may be modified to optimize the biological effect of the one or more DNMT inhibitors and/or the biological effect of the combination of the one or more DNMT inhibitors and Compound A2 or a pharmaceutically acceptable salt, polymorph, solvate, or stereoisomer thereof.

In one aspect, Compound A2 or a pharmaceutically acceptable salt, polymorph, solvate, or stereoisomer thereof and one or more DNMT inhibitors are administered sequentially (e.g., a DNMT inhibitor first then Compound A2). It should be appreciated that Compound A2 or a pharmaceutically acceptable salt, polymorph, solvate, or stereoisomer thereof may be administered according to any of the methods described herein, such as by continuous administration, and/or administration without a drug holiday, after the administration of the one or more DNMT inhibitors. As also described above, a subject may be sensitized or primed by the administration of one or more DNMT inhibitors. In some embodiments, Compound A2 or a pharmaceutically acceptable salt, polymorph, solvate, or stereoisomer thereof is administered with continuous administration, and/or administration without a drug holiday and the one or more DNMT inhibitors are administered one or more days prior to the administration of Compound A2 or a pharmaceutically acceptable salt, polymorph, solvate, or stereoisomer thereof.

In some embodiments, Compound A2 or a pharmaceutically acceptable salt, polymorph, solvate, or stereoisomer thereof is administered with continuous administration, and/or administration without a drug holiday until a desirable biological effect is achieved (e.g., altered chromatin state, reduction of H3K79 methyl mark, and/or cell differentiation) prior to administration of the one or more DNMT inhibitors.

In some embodiments, one or more DNMT inhibitors are administered as indicated on label until a desirable biological effect is achieved (e.g., altered chromatin state, reduction of H3K79 methyl mark, and/or cell differentiation) prior to administration of Compound A2 or a pharmaceutically acceptable salt, polymorph, solvate, or stereoisomer thereof or the combination comprising Compound A2 or a pharmaceutically acceptable salt, polymorph, solvate, or stereoisomer thereof and one or more DNMT inhibitors.

In some embodiments, a subject is evaluated after one treatment regimen described herein for any biological effects. In some embodiments, no further treatment is required if the evaluated biological effect has reached a certain predetermined level or activity. In some embodiments, the biological effect is maturation or induction of blast cells, apoptosis of leukemic blast cells, resolution of fever, cachexia or leukemia cutis, restoration of normal haemoatopoiesis, and/or complete remission. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the treatment.

“Combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. Therapeutic agents may also be administered in alternation.

The combination therapies featured in the present disclosure can result in a synergistic effect in the treatment of a disease or cancer. A “synergistic effect” is defined as where the efficacy of a combination of therapeutic agents is greater than the sum of the effects of any of the agents given alone. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

As provided herein, the administration of the combination of Compound A2 and one or more DNMT inhibitors provides synergistic effects. As provided herein, the combination of Compound A2 and a DNMT inhibitor result in a synergistic antiproliferative response, a synergistic induction of apoptosis in leukemic cells and a synergistic induction of differentiation of leukemic cells. As provided herein synergistic effects also result when leukemic cells are sensitized by the administration of a first DNMT inhibitor prior to the administration of Compound A2 or a combination of Compound A2 and a second DNMT inhibitor.

“Combination therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In another aspect, a combination therapy of the present disclosure may be administered in combination with radiation therapy. Radiation therapy can also be administered in combination with a combination therapy of the present invention and another chemotherapeutic agent described herein as part of a multiple agent therapy.

In one aspect, the present invention also provides pharmaceutical compositions comprising Compound A2 or pharmaceutically acceptable salts thereof, and one or more DNMT inhibitors, mixed with pharmaceutically suitable carriers or excipient(s) at doses to treat or prevent a disease or condition as described herein.

The pharmaceutical compositions of the present invention can also be administered in combination with other therapeutic agents or therapeutic modalities simultaneously, sequentially, or in alternation.

Mixtures of compositions of the present invention can also be administered to the patient as a simple mixture or in suitable formulated pharmaceutical compositions.

A “pharmaceutical composition” is a formulation containing the compounds disclosed herein in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition disclosed herein is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition disclosed herein can be administered to a subject in many of the well-known methods currently used for chemotherapeutic treatment. For example, for treatment of cancers, a compound disclosed herein may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

For any compound disclosed herein, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms disclosed herein is dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

In some embodiments, Compound A2 or a pharmaceutically acceptable salt, polymorph, solvate, or stereoisomer thereof is administered continuously for at least 7, 14, 21, 28, 35, 42, 47, 56, or 64 days in combination with one or more DNMT inhibitors. In some embodiments, Compound A2 or a pharmaceutically acceptable salt, polymorph, solvate, or stereoisomer thereof is administered continuously for at least 7, 14, 21, 28, 35, 42, 47, 56, or 64 days without a drug holiday in combination with one or more DNMT inhibitors.

In some embodiments, Compound A2 or a pharmaceutically acceptable salt, polymorph, solvate, or stereoisomer thereof is administered at a dose of at least 12, 24, 36, 45, 54, 70, 80, or 90 mg/m²/day in combination with one or more DNMT inhibitors. In some embodiments, Compound A2 or a pharmaceutically acceptable salt, polymorph, solvate, or stereoisomer thereof is administered at a dose of at least 12, 24, 36, 45, 54, 70, 80, or 90 mg/m²/day continuously for at least 7, 14, 21, 28, 35, 42, 47, 56, or 64 days in combination with the one or more DNMT inhibitors. In some embodiments, Compound A2 or a pharmaceutically acceptable salt, polymorph, solvate, or stereoisomer thereof is administered at a dose of at least 12, 24, 36, 45, 54, 70, 80, or 90 mg/m²/day continuously without a drug holiday in combination with one or more DNMT inhibitors. In some embodiments, Compound A2 or a pharmaceutically acceptable salt, polymorph, solvate, or stereoisomer thereof is administered at a dose of at least 12, 24, 36, 45, 54, 70, 80, or 90 mg/m²/day continuously for at least 7, 14, 21, 28, 35, 42, 47, 56, or 64 days without a drug holiday in combination with one or more DNMT inhibitors.

In some embodiments, the DNMT inhibitor is Azacitidine. In certain embodiments, Azacitidine is administered at a dosage of 100 mg/m²/day or less (e.g., 75 mg/m²/day or less, 50 mg/m²/day or less, 25 mg/m²/day or less, 15 mg/m²/day or less, 10 mg/m²/day or less, or 5 mg/m²/day or less, or 1-100 mg/m²/day, 5-75 mg/m²/day, 1-5 mg/m²/day, 1-10 mg/m²/day, 1-15 mg/m²/day, 1-25 mg/m²/day, 1-50 mg/m²/day, or 1-75 mg/m²/day) prior to administration of Compound A2. In certain embodiments, Azacitidine is administered at a dosage of 100 mg/m²/day or less (e.g., 75 mg/m²/day or less, 50 mg/m²/day or less, 25 mg/m²/day or less, 15 mg/m²/day or less, 10 mg/m²/day or less, or 5 mg/m²/day or less, or 1-100 mg/m²/day, 5-75 mg/m²/day, 1-5 mg/m²/day, 1-10 mg/m²/day, 1-15 mg/m²/day, 1-25 mg/m²/day, 1-50 mg/m²/day, or 1-75 mg/m²/day) continuously (with or without a drug holiday) for at least 2, 3, 4, 5, 6, 7, 14, 21, 28 days prior to administration of Compound A2. For example, Azacitidine is administered at a dosage of 100 mg/m²/day or less (e.g., 75 mg/m²/day or less, 50 mg/m²/day or less, 25 mg/m²/day or less, 15 mg/m²/day or less, 10 mg/m²/day or less, or 5 mg/m²/day or less, or 1-100 mg/m²/day, 5-75 mg/m²/day, 1-5 mg/m²/day, 1-10 mg/m²/day, 1-15 mg/m²/day, 1-25 mg/m²/day, 1-50 mg/m²/day, or 1-75 mg/m²/day) continuously without a drug holiday for at least 2, 3, 4, 5, 6, or 7 days prior to administration of Compound A2. For example, Azacitidine is administered at a dosage of 100 mg/m²/day or less (e.g., 75 mg/m²/day or less, 50 mg/m²/day or less, 25 mg/m²/day or less, 15 mg/m²/day or less, 10 mg/m²/day or less, or 5 mg/m²/day or less, or 1-100 mg/m²/day, 5-75 mg/m²/day, 1-5 mg/m²/day, 1-10 mg/m²/day, 1-15 mg/m²/day, 1-25 mg/m²/day, 1-50 mg/m²/day, or 1-75 mg/m²/day) for 7 days every 4 weeks prior to administration of Compound A2. For example, Azacitidine is administered at a dosage of 100 mg/m²/day or less (e.g., 75 mg/m²/day or less, 50 mg/m²/day or less, 25 mg/m²/day or less, 15 mg/m²/day or less, 10 mg/m²/day or less, or 5 mg/m²/day or less, or 1-100 mg/m²/day, 5-75 mg/m²/day, 1-5 mg/m²/day, 1-10 mg/m²/day, 1-15 mg/m²/day, 1-25 mg/m²/day, 1-50 mg/m²/day, or 1-75 mg/m²/day) for 1, 2, or 3 days every week (i.e., with a 6-, 5-, or 4-day drug holiday in a 7-day cycle) prior to administration of Compound A2.

The pharmaceutical compositions can be included in a container, pack, or dispenser together with instructions for administration.

The compounds disclosed herein are capable of further forming salts. All of these forms are also contemplated within the scope of the claimed invention.

As used herein, “pharmaceutically acceptable salts” refer to derivatives of the compounds disclosed herein wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, sebacic, subacetic, succinic, sulfamic, sulfanilic, sulfuric, tannic, tartaric, toluene sulfonic, and the commonly occurring amino acids, e.g., glycine, alanine, phenylalanine, arginine, etc.

Other examples of pharmaceutically acceptable salts include hexanoic acid, cyclopentenyl propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compounds disclosed herein can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds disclosed herein can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds disclosed herein can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug disclosed herein in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds disclosed herein wherein a hydroxy, amino, sulfhydryl, carboxyl or carbonyl group is bonded to any group that may be cleaved in vivo to form a free hydroxyl, free amino, free sulfhydryl, free carboxyl or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds disclosed herein, and the like, See Bundegaard, H., Design of Prodrugs, p1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

Compounds described herein are assayed for modulation of activity, for example, histone methylation, modulation of cell growth and/or IC₅₀, described in the examples below or those described in co-pending applications 62/051,890 filed on Sep. 17, 2014, 62/088,498 filed on Dec. 5, 2014, and PCT/US2014/065517 filed on Nov. 13, 2014, and WO 2014/153001, the contents of each of which are hereby incorporated by reference in their entireties. Diseases such as cancers and neurological disease can be treated by administration of modulators of protein (e.g., histone) methylation, e.g., modulators of histone methyltransferase, or histone demethylase enzyme activity. Histone methylation has been reported to be involved in aberrant expression of certain genes in cancers, and in silencing of neuronal genes in non-neuronal cells. The composition or combination of compounds of this invention, e.g. a combination comprising Compound A2 or a pharmaceutically acceptable salt, polymorph, solvate, or stereoisomer thereof and one or more DNMT inhibitors described herein can be used to treat such diseases, i.e., to decrease or inhibit methylation of histones in affected cells or restore methylation to roughly its level in counterpart normal cells.

The present invention provides compositions, combinations of compounds disclosed herein and methods for treating or alleviating a symptom of conditions and diseases the course of which can be influenced by modulating the methylation status of histones or other proteins, wherein said methylation status is mediated at least in part by the activity of DOT1L. Modulation of the methylation status of histones can in turn influence the level of expression of target genes activated by methylation, and/or target genes suppressed by methylation. The method includes administering to a subject in need of such treatment, a therapeutically effective amount of a DNMT inhibitor prior to administration of a therapeutically effective amount of Compound A2 or a combination of Compound A2 and a DNMT inhibitor to a subject in need of such treatment.

Modulators of methylation can be used for modulating cell proliferation, generally. For example, in some cases excessive proliferation may be reduced with agents that decrease methylation, whereas insufficient proliferation may be stimulated with agents that increase methylation. Accordingly, diseases that may be treated include hyperproliferative diseases, such as benign cell growth and malignant cell growth (cancer).

The disorder in which DOT1L-mediated protein methylation plays a part can be cancer, a cell proliferative disorder, or a precancerous condition. Exemplary cancers that may be treated include brain and CNS cancer, kidney cancer, ovarian cancer, pancreatic cancer, lung cancer, breast cancer, colon cancer, prostate cancer, or a hematological cancer. For example, the hematological cancer is leukemia or lymphoma. Preferably the cancer is leukemia. The leukemia can be acute or chronic leukemia. In some embodiments, the leukemia is acute myeloid leukemia (AML) or acute lymphocytic leukemia. In some embodiments, leukemia that may be treated is leukemia characterized by a chromosomal rearrangement on chromosome 11q23, including chimeric fusion of mixed lineage leukemia gene (MLL) or partial tandem duplication of MLL (MLL-PTD). In some embodiments, leukemia that may be treated is leukemia characterized by the presence of a genetic lesion of MLL. Such genetic lesions include chromosomal rearrangements, such as translocations, deletions, and/or duplications of the MLL gene. MLL has been categorized or characterized as having a chimeric fusion of MLL, partial tandem duplication of the MLL gene (MLL-PTD), or non-rearranged MLL.

The disorder that can be treated by the combination therapy described herein can be a disorder medicated by translocation, deletion and/or duplication of a gene on chromosome 11q23.

In general, compounds that are methylation modulators can be used for modulating cell proliferation. For example, in some cases excessive proliferation may be reduced with agents that decrease methylation, whereas insufficient proliferation may be stimulated with agents that increase methylation. Accordingly, diseases that may be treated by the compounds disclosed herein include hyperproliferative diseases, such as benign cell growth and malignant cell growth.

As used herein, a “subject in need thereof” is a subject having a disorder in which DOT1L-mediated protein methylation plays a part, or a subject having an increased risk of developing such disorder relative to the population at large. A subject in need thereof can have a precancerous condition. Preferably, a subject in need thereof has cancer. A “subject” includes a mammal. The mammal can be e.g., any mammal, e.g., a human, primate, bird, mouse, rat, fowl, dog, cat, cow, horse, goat, camel, sheep or pig. Preferably, the mammal is a human.

In some embodiments, the subject is child. In some embodiments, the subject is younger than 18 years of age. In some embodiments, the subject is younger than 18, 17, 16, 15, 14, 13, 12, 11, 10, 9, 8, 7, 6, 5, 4, 3, 2, or 1 year of age. In some embodiments, the subject is between 3 months and 18 years of age.

The subject of the present disclosure includes any human subject who has been diagnosed with, has symptoms of, or is at risk of developing a cancer or a precancerous condition.

A subject in need thereof may be a subject having a disorder associated DOT1L. A subject in need thereof can have a precancerous condition. Preferably, a subject in need thereof has cancer. A subject in need thereof can have cancer associated with DOT1L. In a preferred aspect, a subject in need thereof has one or more cancers selected from the group consisting of brain and central nervous system (CNS) cancer, head and neck cancer, kidney cancer, ovarian cancer, pancreatic cancer, leukemia, lung cancer, lymphoma, myeloma, sarcoma, breast cancer, prostate cancer and a hematological cancer. Preferably, a subject in need thereof has a hematologic cancer, wherein the hematologic cancer is leukemia or lymphoma. Exemplary leukemia is MLL. Other hematologic cancers can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A subject in need thereof can be one who has been previously diagnosed or identified as having cancer or a precancerous condition. A subject in need thereof can also be one who is having (suffering from) cancer or a precancerous condition. Alternatively, a subject in need thereof can be one who is having an increased risk of developing such disorder relative to the population at large (i.e., a subject who is predisposed to developing such disorder relative to the population at large).

A subject in need thereof can have cancer associated with increased expression (mRNA or protein) and/or activity level of at least one protein selected from the group consisting of HOXA9, FLT3, MEIS1, MEIS2, TBP, BCL, and DOT1L. A subject in need thereof may have increased mRNA, protein, and/or activity level of at least of at least one signaling component downstream of at least one protein selected from the group consisting of HOXA9, FLT3, MEIS1, MEIS2, TBP, BCL, and DOT1L. Such downstream components are readily known in the art, and can include other transcription factors, or signaling proteins. As used herein, the term “increase in activity” refers to increased or a gain of function of a gene product/protein compared to the wild type. Accordingly, an increase in mRNA or protein expression and/or activity levels can be detected using any suitable method available in the art.

Optionally a subject in need thereof has already undergone, is undergoing or will undergo, at least one therapeutic intervention for the cancer or precancerous condition.

A subject in need thereof may have refractory cancer on most recent therapy. “Refractory cancer” means cancer that does not respond to treatment. The cancer may be resistant at the beginning of treatment or it may become resistant during treatment. Refractory cancer is also called resistant cancer. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy.

In some embodiments, a subject in need thereof may have a secondary cancer as a result of a previous therapy. “Secondary cancer” means cancer that arises due to or as a result from previous carcinogenic therapies, such as chemotherapy. In some embodiments, the secondary cancer is a hematologic cancer, such as leukemia.

The subject may exhibit resistance to DOT1L histone methyltransferase inhibitors or any other therapeutic agent.

The disclosure also features a method of selecting a combination therapy for a subject having leukemia. The method includes the steps of: detecting the level of HOXA9, FLT3, MEIS1, MEIS2, TBP, BCL, and/or DOT1L in a sample from the subject; and selecting, based on the presence of the increased level of HOXA9, FLT3, MEIS1, MEIS2, TBP, BCL, and/or DOT1L, a combination therapy for treating leukemia. In one embodiment, the therapy includes administering to the subject a composition or combination of compounds of the disclosure. In one embodiment, the method further includes administrating to the subject a therapeutically effective amount of a DNMT inhibitor prior to administration of Compound A2 or a combination of the disclosure comprising a DNMT inhibitor and Compound A2. In one embodiment, the leukemia is characterized by partial tandem duplication of the MLL gene (MLL-PTD)n. In another embodiment, the leukemia is characterized by overexpression of HOXA9, FLT3, MEIS1, MEIS2, TBP, BCL, and/or DOT1L.

The methods and uses described herein may include steps of detecting the mRNA, protein and/or activity (function) level of HOXA9, FLT3, MEIS1, MEIS2, TBP, BCL, and/or DOT1L in a sample from a subject in need thereof prior to and/or after the administration of a combination of the disclosure (e.g., administration of a DNMT inhibitor prior to administration of Compound A2 or a combination of the disclosure comprising a DNMT inhibitor and Compound A2) to the subject. The presence of the increased level of HOXA9, FLT3, MEIS1, MEIS2, TBP, BCL, and/or DOT1L in the tested sample indicates the subject is responsive to the combination therapy described herein.

The present disclosure provides personalized medicine, treatment and/or cancer management for a subject by genetic screening of increased gene expression (mRNA or protein), and/or increased function or activity level of at least one protein selected from the group consisting of HOXA9, FLT3, MEIS1, MEIS2, TBP, BCL, and DOT1L in the subject. For example, the present disclosure provides methods for treating or alleviating a symptom of cancer or a precancerous condition in a subject in need thereof by determining responsiveness of the subject to a combination therapy and when the subject is responsive to the combination therapy, administering to the subject a combination of compounds of the disclosure. The responsiveness is determined by obtaining a sample from the subject and detecting increased mRNA or protein, and/or increased activity level of at least one protein selected from the group consisting of HOXA9, FLT3, MEIS1, MEIS2, TBP, BCL, and DOT1L, and the presence of such gain of expression and/or function indicates that the subject is responsive to the combination therapy of the disclosure. Once the responsiveness of a subject is determined, a therapeutically effective amount of a combination of the disclosed compounds, for example, a combination comprising Compound A2 or a pharmaceutically acceptable salt, polymorph, solvate, or stereoisomer thereof, and one or more DNMT inhibitors, can be administered. The therapeutically effective amount of a combination therapy can be determined by one of ordinary skill in the art.

As used herein, the term “responsiveness” is interchangeable with terms “responsive”, “sensitive”, and “sensitivity”, and it is meant that a subject is showing therapeutic responses when administered a combination therapy of the invention, e.g., tumor cells or tumor tissues of the subject undergo apoptosis and/or necrosis, and/or display reduced growing, dividing, or proliferation. This term is also meant that a subject will or has a higher probability, relative to the population at large, of showing therapeutic responses when administered a combination therapy of the invention, e.g., tumor cells or tumor tissues of the subject undergo apoptosis and/or necrosis, and/or display reduced growing, dividing, or proliferation.

By “sample” it means any biological sample derived from the subject, includes but is not limited to, cells, tissues samples, body fluids (including, but not limited to, mucus, blood, plasma, serum, urine, saliva, and semen), tumor cells, and tumor tissues. Preferably, the sample is selected from bone marrow, peripheral blood cells, blood, plasma and serum. Samples can be provided by the subject under treatment or testing. Alternatively samples can be obtained by the physician according to routine practice in the art.

An increase in mRNA or protein expression and/or activity levels can be detected using any suitable method available in the art. For example, an increase in activity level can be detected by measuring the biological function of a gene product, such as the histone methyltransferase activity of DOT1L (i.e., methylation of histone substrates such as H3K79 by immunoblot); transcriptional activity of HOXA9, MEIS2 or MEIS1 (i.e., expression levels of HOXA9, MEIS2 or MEIS1 target genes by RT-PCR); or phosphorylation activity of FLT3 (i.e., phosphorylation status of FLT3 targets by immunoblot or radioimmunoassay). Alternatively, a gain of function mutation can be determined by detecting any alternation in a nucleic acid sequence encoding a protein selected from the group consisting of HOXA9, FLT3, MEIS1, MEIS2, TBP, BCL, and DOT1L. For example, a nucleic acid sequence encoding HOXA9, FLT3, MEIS1, MEIS2, TBP, BCL, and/or DOT1L having a gain of function mutation can be detected by whole-genome resequencing or target region resequencing (the latter also known as targeted resequencing) using suitably selected sources of DNA and polymerase chain reaction (PCR) primers in accordance with methods well known in the art. The method typically and generally entails the steps of genomic DNA purification, PCR amplification to amplify the region of interest, cycle sequencing, sequencing reaction cleanup, capillary electrophoresis, and/or data analysis. Alternatively or in addition, the method may include the use of microarray-based targeted region genomic DNA capture and/or sequencing. Kits, reagents, and methods for selecting appropriate PCR primers and performing resequencing are commercially available, for example, from Applied Biosystems, Agilent, and NimbleGen (Roche Diagnostics GmbH). Detection of mRNA expression can be detected by methods known in the art, such as Northern blot, nucleic acid PCR, and quantitative RT-PCR. Detection of polypeptide expression (i.e., wild-type or mutant) can be carried out with any suitable immunoassay in the art, such as Western blot analysis.

As used herein, the term “cell proliferative disorder” refers to conditions in which unregulated or abnormal growth, or both, of cells can lead to the development of an unwanted condition or disease, which may or may not be cancerous. Exemplary cell proliferative disorders encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue.

A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; pulmonary sarcoidosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute lymphocytic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Preferably, compositions or combinations of the present disclosure may be used to treat a cancer selected from the group consisting of a hematologic cancer or a hematologic cell proliferative disorder. A hematologic cancer can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, compositions or combinations of the present disclosure may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioloalveolar carcinoma, giant cell carcinoma, spindle cell carcinoma, and large cell neuroendocrine carcinoma. Lung cancer can include lung neoplasms having histologic and ultrastructural heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer. Preferably, compositions or combinations of the present disclosure may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructural heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions or combinations of the present disclosure may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Preferably, compositions or combinations of the present disclosure may be used to treat breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphocytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructural heterogeneity (e.g., mixed cell types).

Preferably, compound disclosed herein, or a pharmaceutically acceptable salt, polymorph, or solvate thereof, may be used to treat breast cancer. A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich, or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound disclosed herein, or a pharmaceutically acceptable salt, polymorph or solvate thereof, may be used to treat or prevent a cell proliferative disorder of the breast, or to treat or prevent breast cancer, in a subject having an increased risk of developing breast cancer relative to the population at large. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can histologically graded according to the Scarff-Bloom-Richardson system, wherein a breast tumor has been assigned a mitosis count score of 1, 2, or 3; a nuclear pleomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pNO, PNO (I−), PNO (I+), PNO (mol−), PNO (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

The compound disclosed herein, or a pharmaceutically acceptable salt, polymorph or solvate thereof, has been or will be tested in one or more in vitro or in vivo biological assays, in order to determine if that compound is likely to elicit a desired biological or medical response in a cell, tissue, system, animal or human that is being sought by a researcher or clinician. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

For example, an in vitro biological assay that can be used includes the steps of (1) mixing a histone substrate (e.g., an isolated histone sample for a histone or modified histone of interest, or an isolated oligonucleosome substrate) with recombinant DOT1L enzyme (e.g., recombinant protein containing amino acids 1-416); (2) adding a compound to this mixture; (3) adding non-radioactive and ³H-labeled S-Adenosyl methionine (SAM) to start the reaction; (4) adding excessive amount of non-radioactive SAM to stop the reaction; (4) washing off the free non-incorporated ³H-SAM; and (5) detecting the quantity of ³H-labeled histone substrate by any methods known in the art (e.g., by a PerkinElmer TopCount plate reader).

For example, an in vitro cell viability assay that can be used includes the steps of (1) culturing cells (e.g., EOL-1, KOPM-88, Molm13, MV411, LOUCY, SemK2, Reh, HL60, BV173, or Jurkat cells) in the presence of increasing concentration of a compound (e.g., Compound A2); (2) determining viable cell number every 3-4 days by methods known in the art (e.g., using the Millipore Guava Viacount assay); (3) plotting concentration-dependence growth curves; and optionally (4) calculating IC₅₀ values from the concentration-dependence growth curves using methods known in the art (e.g., using GraphPad Prism Software).

For example, a histone methylation assay that can be used includes the steps of (1) culturing cells (e.g., EOL-1, KOPM-88, Molm13, MV411, LOUCY, SemK2, Reh, HL60, BV173, or Jurkat cells) in the presence of a compound (e.g., Compound A2 and/or a DNMT inhibitor); (2) harvesting the cells; (3) extracting histone proteins, using methods known in the art (e.g., sulfuric acid precipitation); (4) fractionating histone extracts by SDS-PAGE electrophoresis and transferring to a filter; (5) probing the filter with antibodies specific to a protein or methylated-protein of interest (e.g., H3K79me2-specific antibody and total histone H3-specific antibody); and (6) detecting the signal of the antibodies using methods known in the art (e.g., Li-cor Odyssey infrared imager).

For example, a gene expression assay that can be used includes the steps of (1) culturing cells (e.g., EOL-1, KOPM-88, Molm13, MV411, LOUCY, SemK2, Reh, HL60, BV173, or Jurkat cells) in the presence or absence of a compound (e.g., Compound A2 and/or a DNMT inhibitor); (2) harvesting the cells; (3) extracting the RNA using methods known in the art (e.g., Qiagen RNeasy Kit); (4) synthesizing cDNA from the extracted RNA (e.g., Applied Biosystems reverse transcriptase kit); (5) preparing qPCR reactions using, for example, primers and probes (e.g., predesigned labeled primer and probe sets for HOXA9, FLT3, MEIS1, MEIS2, TBP, BCL, DOT1L, and β2-microglobulin from Applied Biosystems), synthesized sample cDNA, and qPCR master mix reagent (e.g., Applied Biosystems Taqman universal PCR master mix); (6) running samples on PCR machine (e.g., Applied Biosystems); (7) analysis of the data and calculation of relative gene expression.

As used herein, “monotherapy” refers to the administration of a single active or therapeutic compound to a subject in need thereof. Preferably, monotherapy will involve administration of a therapeutically effective amount of a single active compound. For example, cancer monotherapy with one of the compound disclosed herein, or a pharmaceutically acceptable salt, analog or derivative thereof, to a subject in need of treatment of cancer. In one aspect, the single active compound is a compound disclosed herein, or a pharmaceutically acceptable salt, polymorph or solvate thereof.

As used herein, “treating” or “treat” describes the management and care of a patient for the purpose of combating a disease, condition, or disorder and includes the administration of one or more compounds disclosed herein, or a pharmaceutically acceptable salt, polymorph or solvate thereof, to alleviate the symptoms or complications of a disease, condition or disorder, or to eliminate the disease, condition or disorder.

A compound disclosed herein, or a pharmaceutically acceptable salt, polymorph or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions disclosed herein leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, at the World Wide Web (www) cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, at the World Wide Web (www) cancer.gov). Furthermore, severity describes a nuclear grade, which refers to the size and shape of the nucleus in tumor cells and the percentage of tumor cells that are dividing (see, National Cancer Institute, at the World Wide Web (www) cancer.gov).

In another aspect, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant disclosure.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present disclosure, or a pharmaceutically acceptable salt, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present disclosure, or a pharmaceutically acceptable salt, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleomorphism.

As used herein, the term “selectively” means tending to occur at a higher frequency in one population than in another population. The compared populations can be cell populations. Preferably, a compound of the present disclosure, or a pharmaceutically acceptable salt, polymorph or solvate thereof, acts selectively on a cancer or precancerous cell but not on a normal cell. Preferably, a compound of the present disclosure, or a pharmaceutically acceptable salt, polymorph or solvate thereof, acts selectively to modulate one molecular target (e.g., a target protein methyltransferase) but does not significantly modulate another molecular target (e.g., a non-target protein methyltransferase). The disclosure also provides a method for selectively inhibiting the activity of an enzyme, such as a protein methyltransferase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A combination therapy of the present disclosure e.g., administration of a DNMT inhibitor prior to administration of Compound A2 or a combination of the disclosure comprising a DNMT inhibitor and Compound A2, can modulate the activity of a molecular target (e.g., a target protein methyltransferase). Modulating refers to stimulating or inhibiting an activity of a molecular target. Preferably, a combination therapy of the disclosure modulates the activity of a molecular target if it stimulates or inhibits the activity of the molecular target by at least 2-fold relative to the activity of the molecular target under the same conditions but lacking only the presence of said compound. More preferably, a composition or combination therapy of the present disclosure modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

As used herein, the term “isozyme selective” means preferential inhibition or stimulation of a first isoform of an enzyme in comparison to a second isoform of an enzyme (e.g., preferential inhibition or stimulation of a protein methyltransferase isozyme alpha in comparison to a protein methyltransferase isozyme beta). Preferably, a composition or a combination therapy of the present disclosure demonstrates a minimum of a fourfold differential, preferably a tenfold differential, more preferably a fifty fold differential, in the dosage required to achieve a biological effect. Preferably, a composition or a combination therapy of the present disclosure demonstrates this differential across the range of inhibition, and the differential is exemplified at the IC₅₀, i.e., a 50% inhibition, for a molecular target of interest.

Administering a composition or a combination of compounds of the present disclosure to a cell or a subject in need thereof can result in modulation (i.e., stimulation or inhibition) of an activity of a protein methyltransferase of interest. Several intracellular targets can be modulated with the compounds of the present invention, including, but not limited to, protein methyltransferase.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. A cell cycle checkpoint regulator can be a protein or not a protein.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

Preferably, an effective amount of a composition or combination of compounds of the present disclosure is not significantly cytotoxic to normal cells. A therapeutically effective amount of a composition is not significantly cytotoxic to normal cells if administration of the composition in a therapeutically effective amount does not induce cell death in greater than 10% of normal cells. A therapeutically effective amount of a composition does not significantly affect the viability of normal cells if administration of the composition in a therapeutically effective amount does not induce cell death in greater than 10% of normal cells. In an aspect, cell death occurs by apoptosis.

Contacting a cell with a composition or combination of compounds disclosed herein can induce or activate cell death selectively in cancer cells. Administering to a subject in need thereof a composition or combination of compounds of the present disclosure can induce or activate cell death selectively in cancer cells. Contacting a cell with a composition or combination of compounds of the present disclosure can induce cell death selectively in one or more cells affected by a cell proliferative disorder. Preferably, administering to a subject in need thereof a composition or combination of compounds of the present disclosure induces cell death selectively in one or more cells affected by a cell proliferative disorder.

The present disclosure relates to a method of treating or alleviating a symptom of cancer by administering a composition or combination of compounds of the present disclosure to a subject in need thereof, where administration of the composition results in one or more of the following: accumulation of cells in G1 and/or S phase of the cell cycle, cytotoxicity via cell death in cancer cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the invention

The composition of the instant disclosure can also be utilized to treat or alleviate a symptom of neurologic diseases or disorders. Neurologic diseases or disorders that may be treated with the compounds of this disclosure include epilepsy, schizophrenia, bipolar disorder or other psychological and/or psychiatric disorders, neuropathies, skeletal muscle atrophy, and neurodegenerative diseases, e.g., a neurodegenerative disease. Exemplary neurodegenerative diseases include: Alzheimer's, Amyotrophic Lateral Sclerosis (ALS), and Parkinson's disease. Another class of neurodegenerative diseases includes diseases caused at least in part by aggregation of poly-glutamine. Diseases of this class include: Huntington's Diseases, Spinalbulbar Muscular Atrophy (SBMA or Kennedy's Disease) Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCAT), and Spinocerebellar Ataxia 12 (SCA12).

Any other disease in which epigenetic methylation, which is mediated by DOT1, plays a role may be treatable or preventable using compounds and methods described herein.

The present disclosure provides use of a composition disclosed herein for inhibiting DOT1L activity in a cell. Still another aspect of the disclosure relates to a use of a composition disclosed herein for reducing the level of methylation of histone H3 lysine residue 79 (H3-K79) in a cell.

Any of the above aspects and embodiments can be combined with any other aspect or embodiment.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLE 1

### DOT1L Combination Studies in MLL-Rearranged Cell Lines

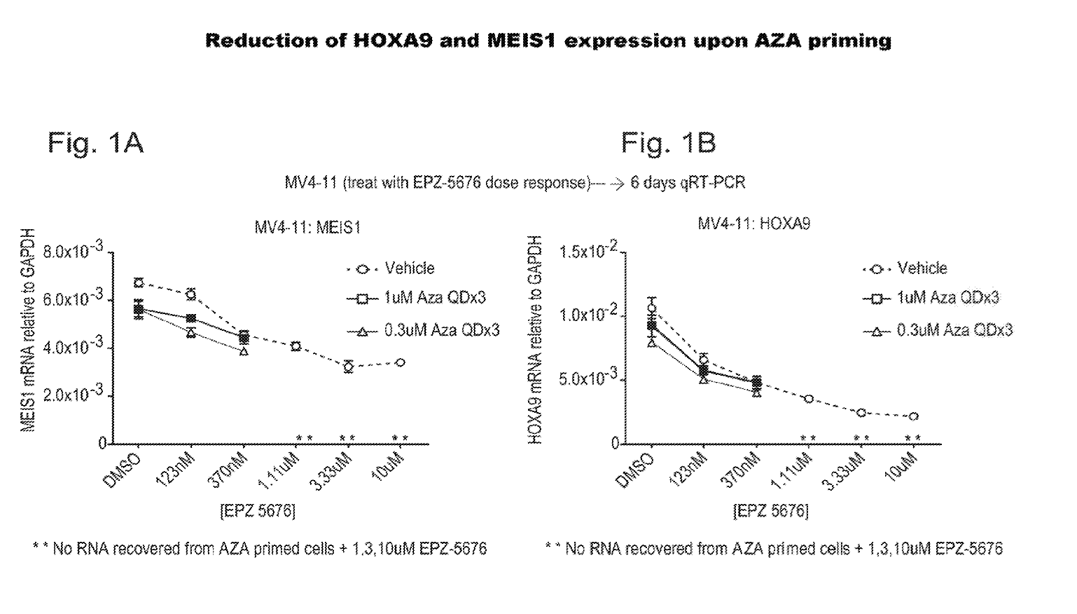

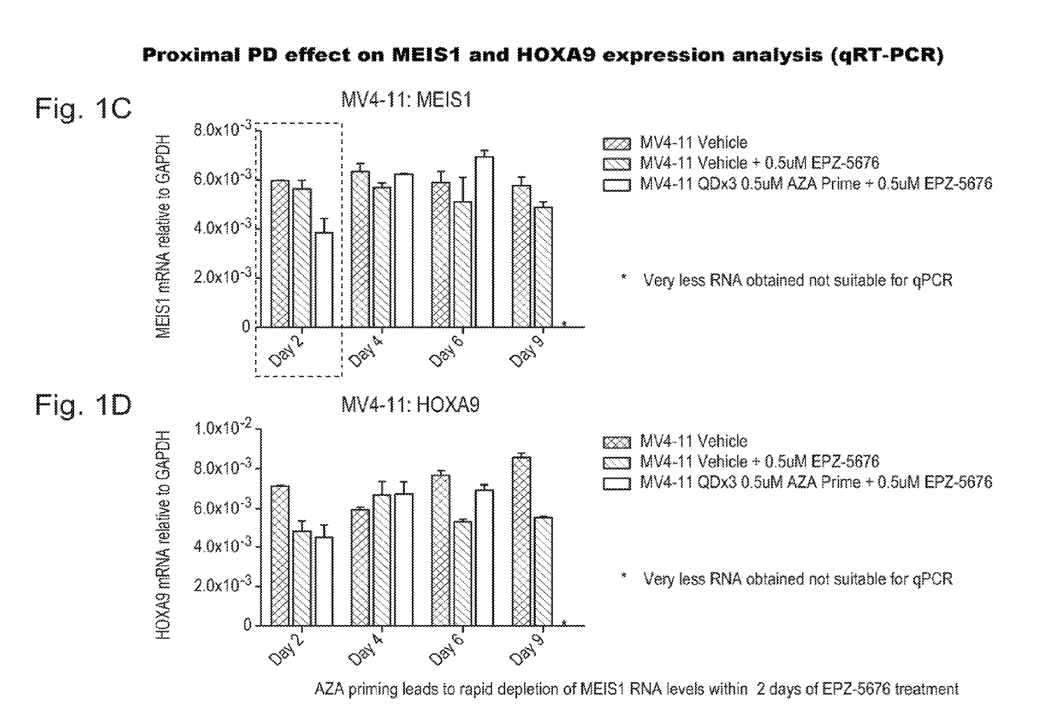

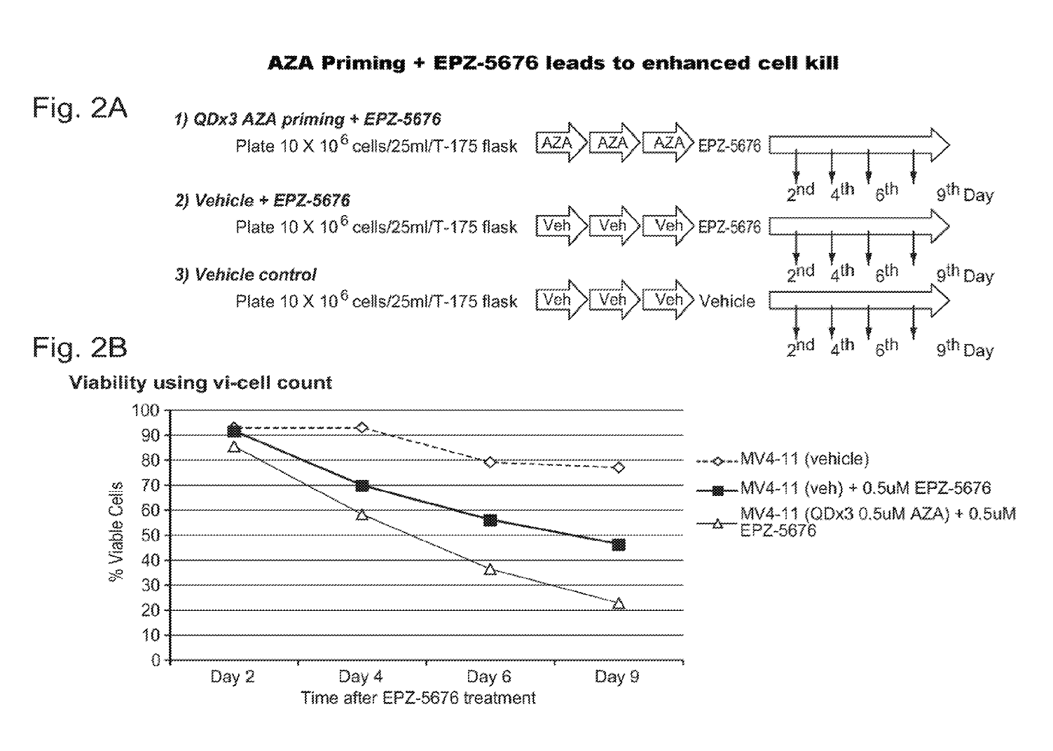

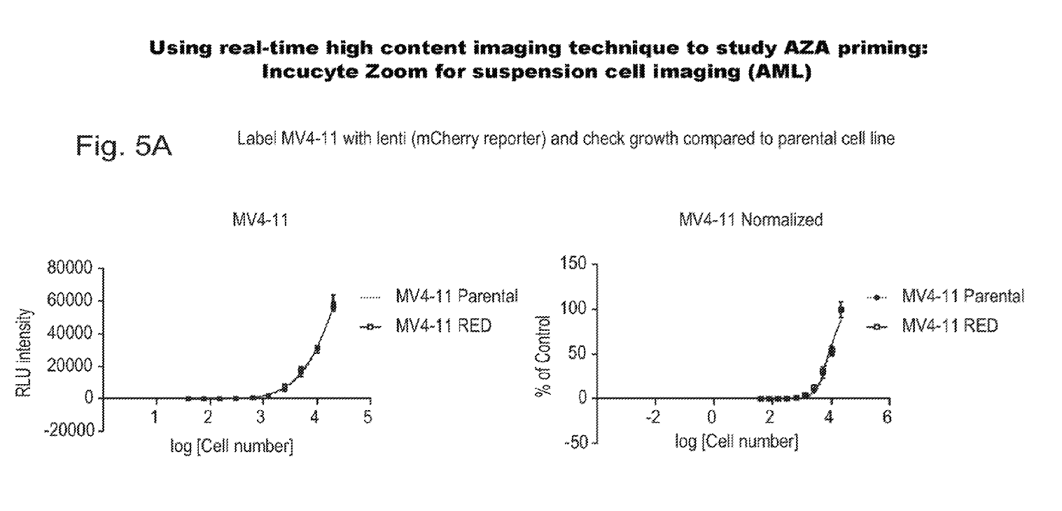

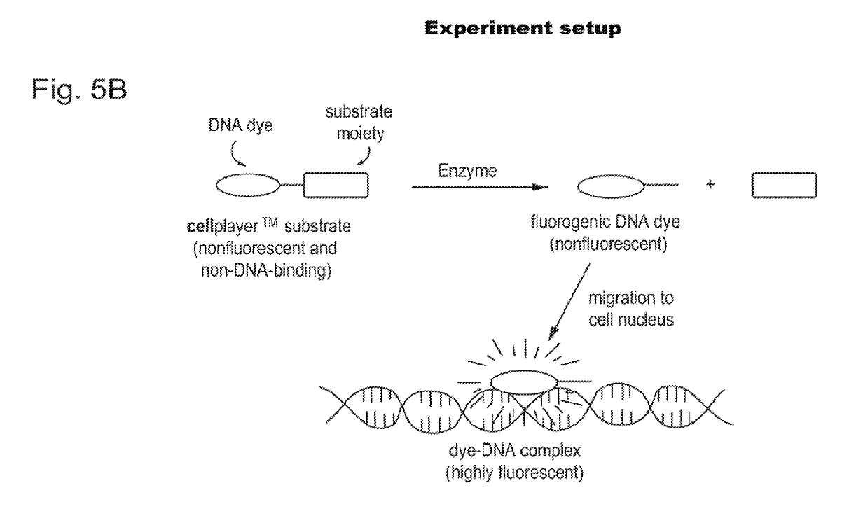

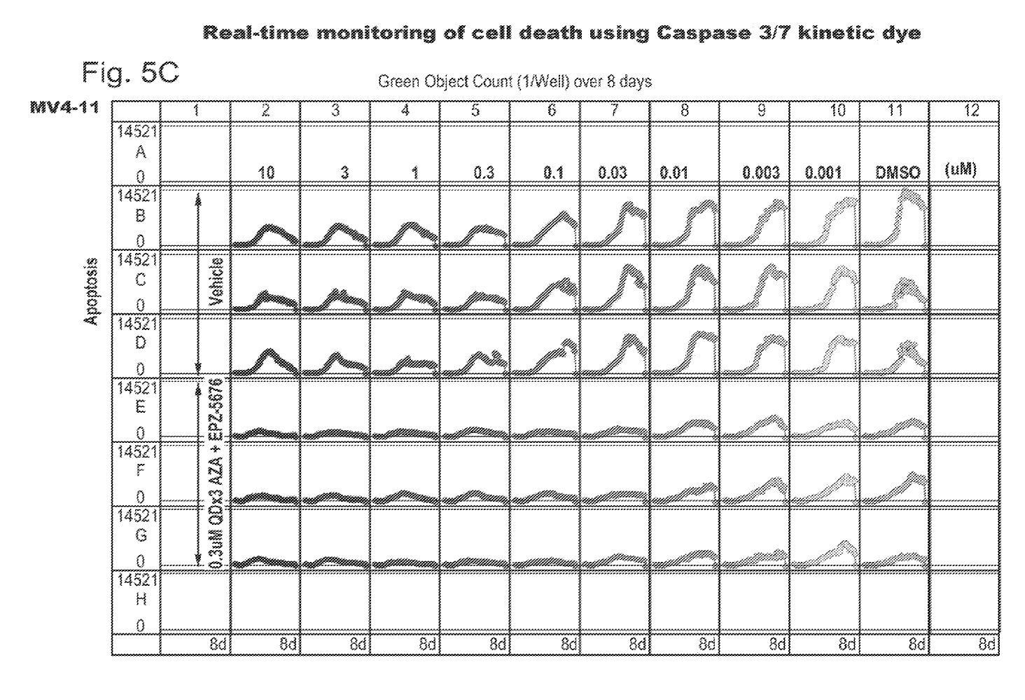

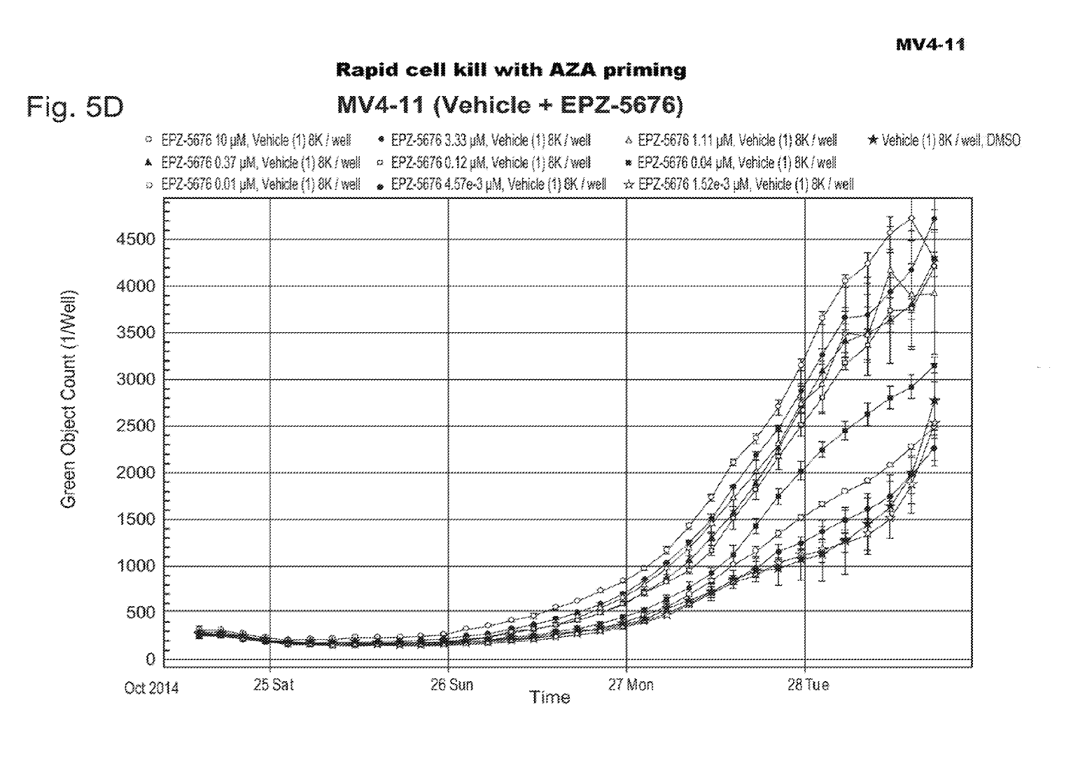

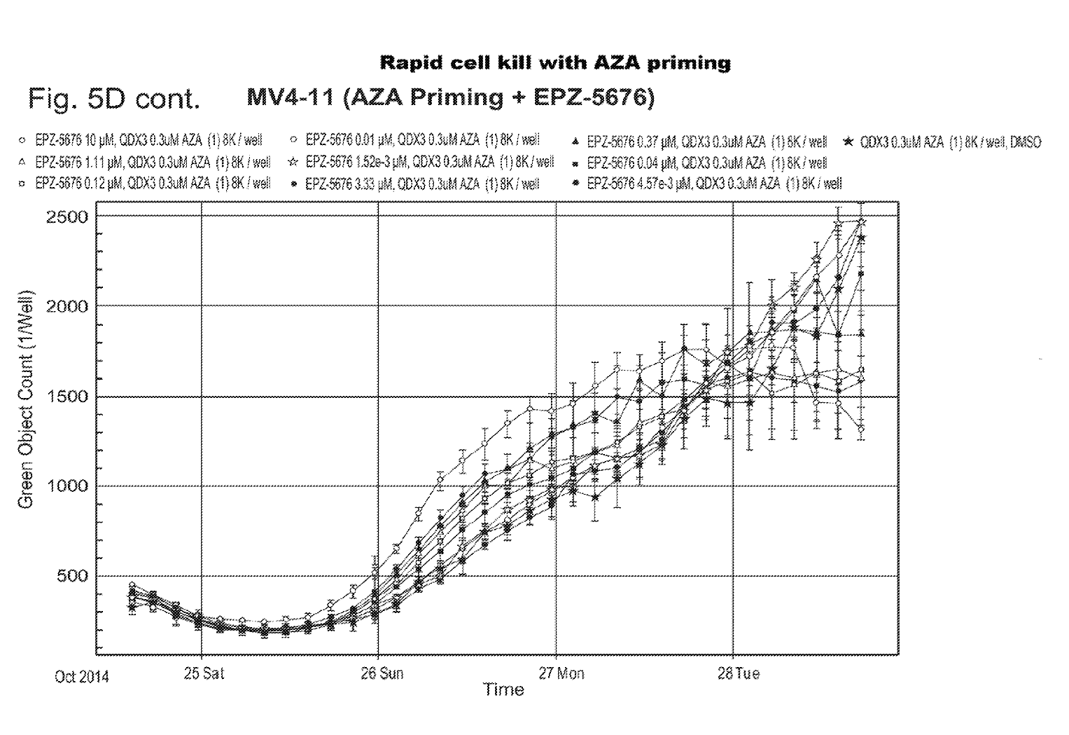

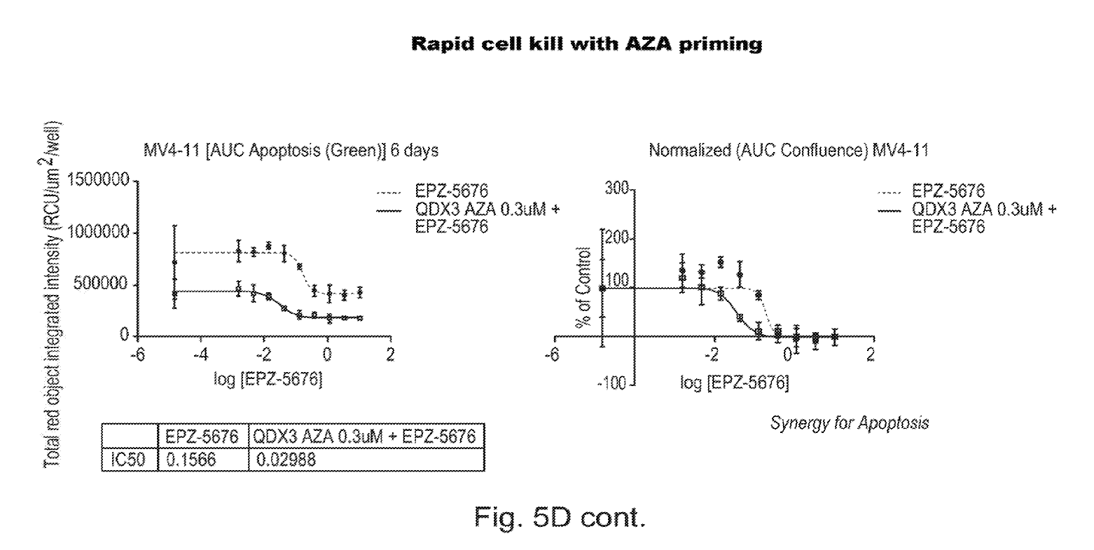

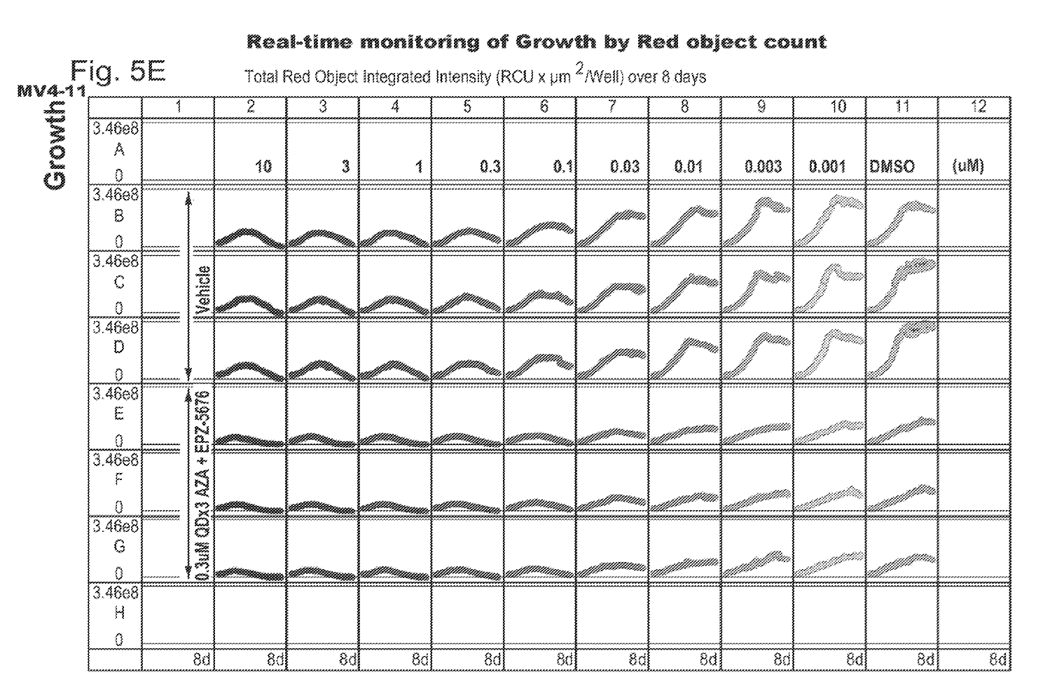

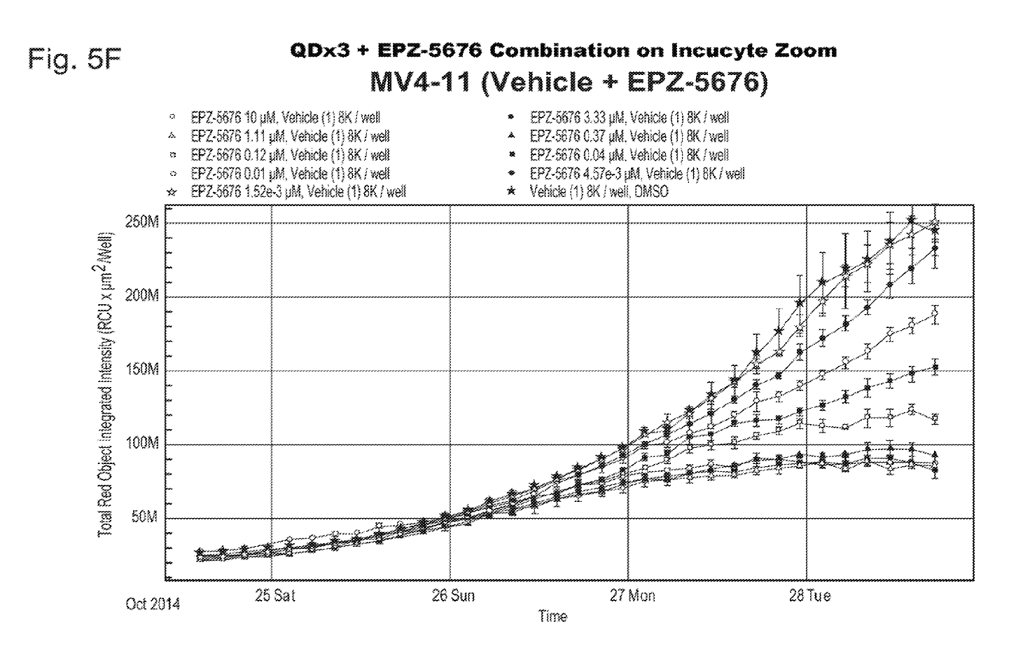

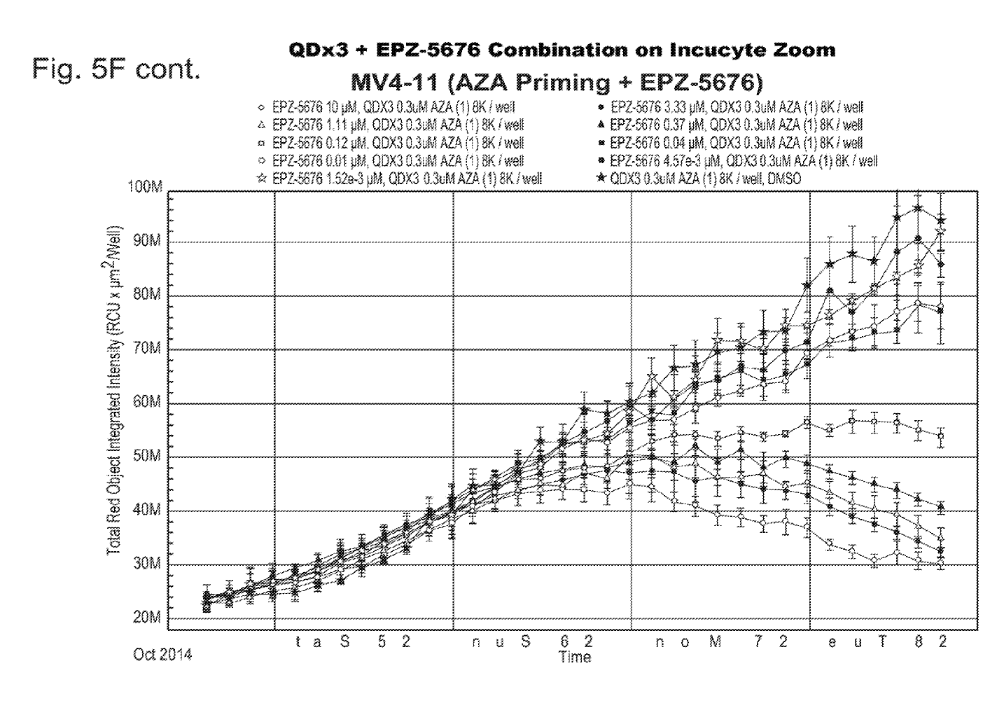

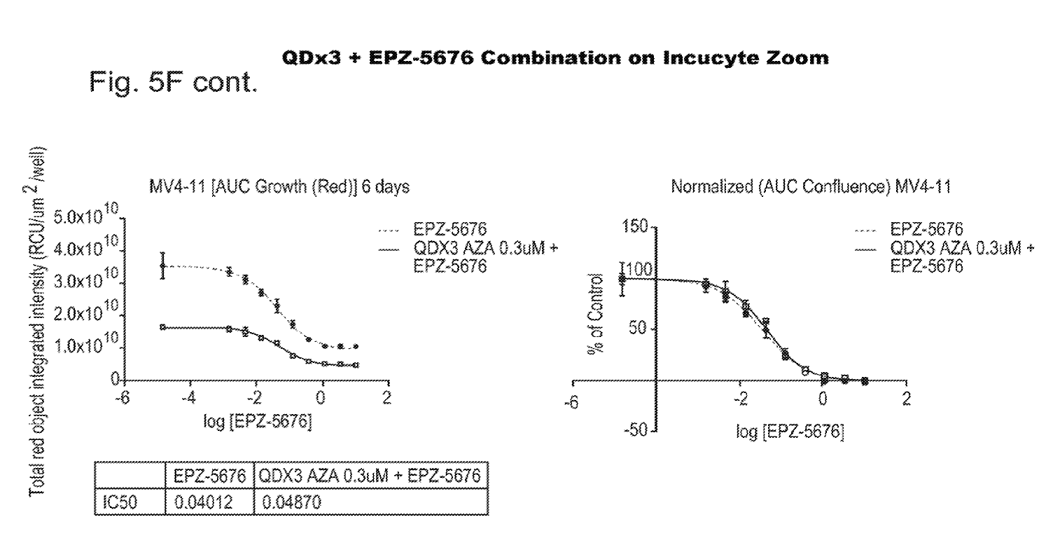

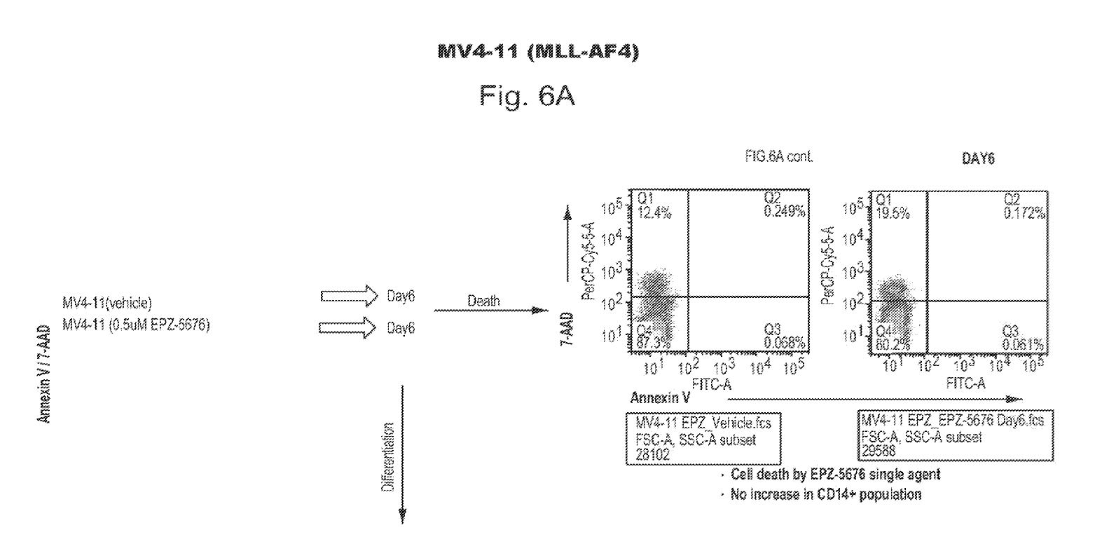

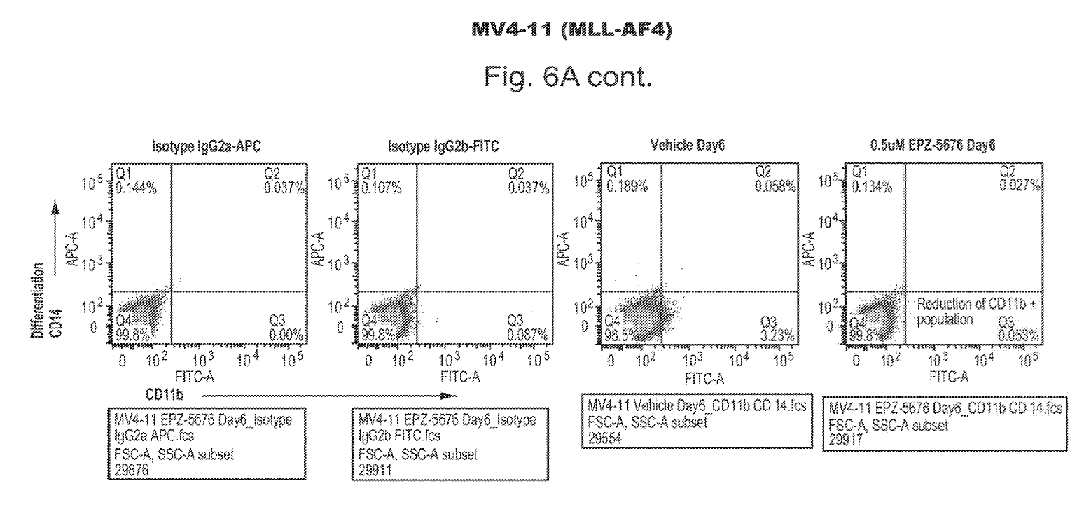

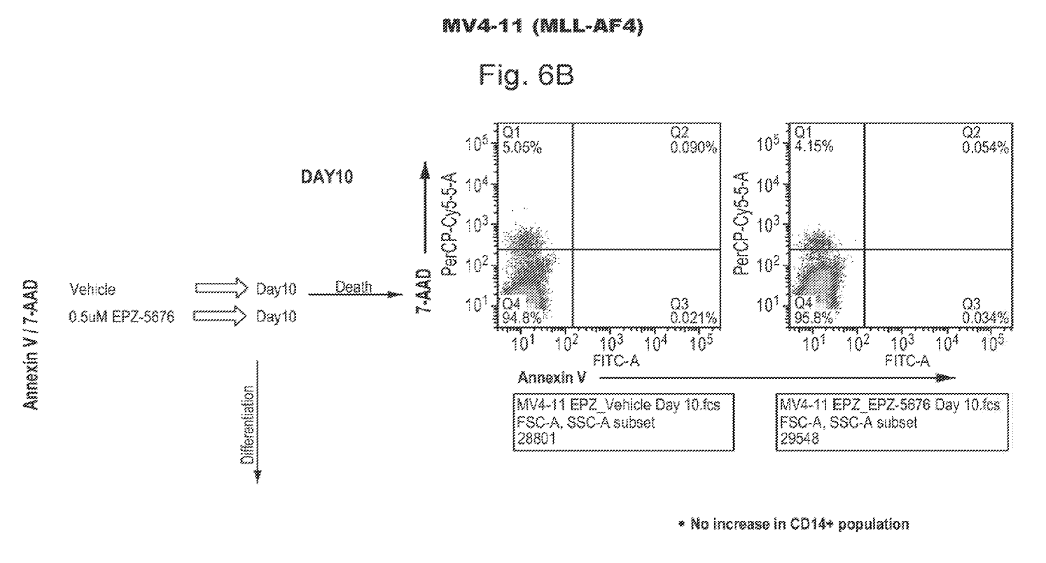

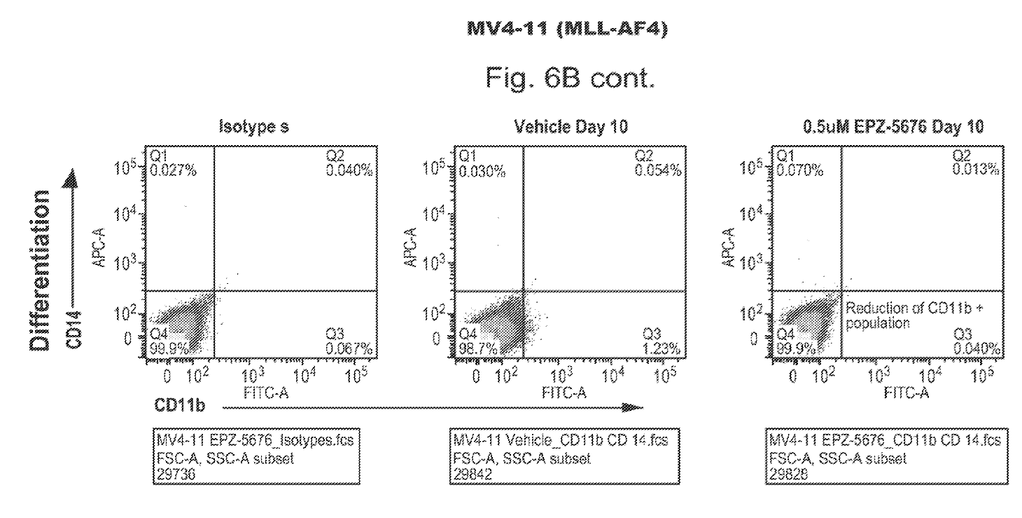

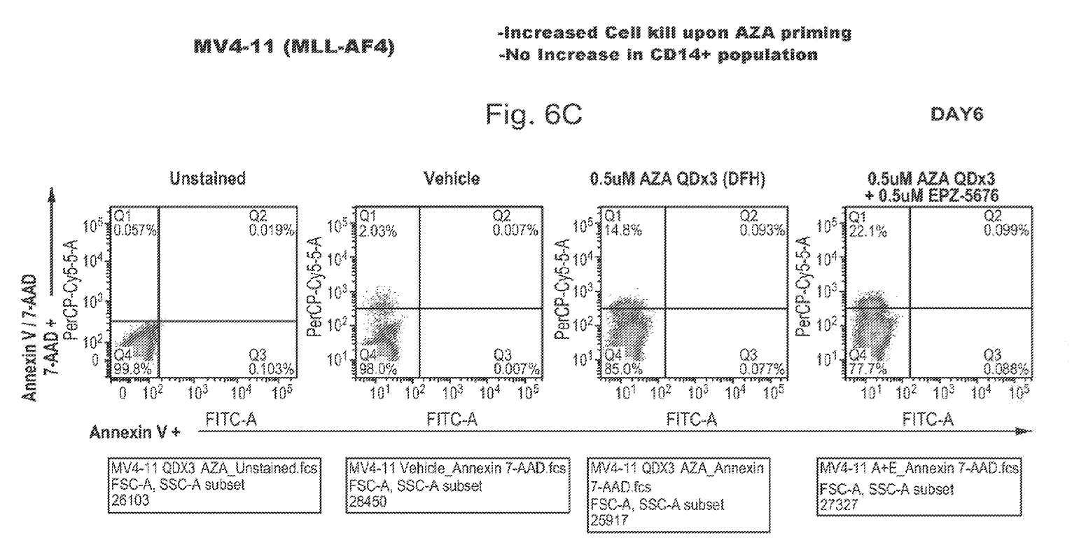

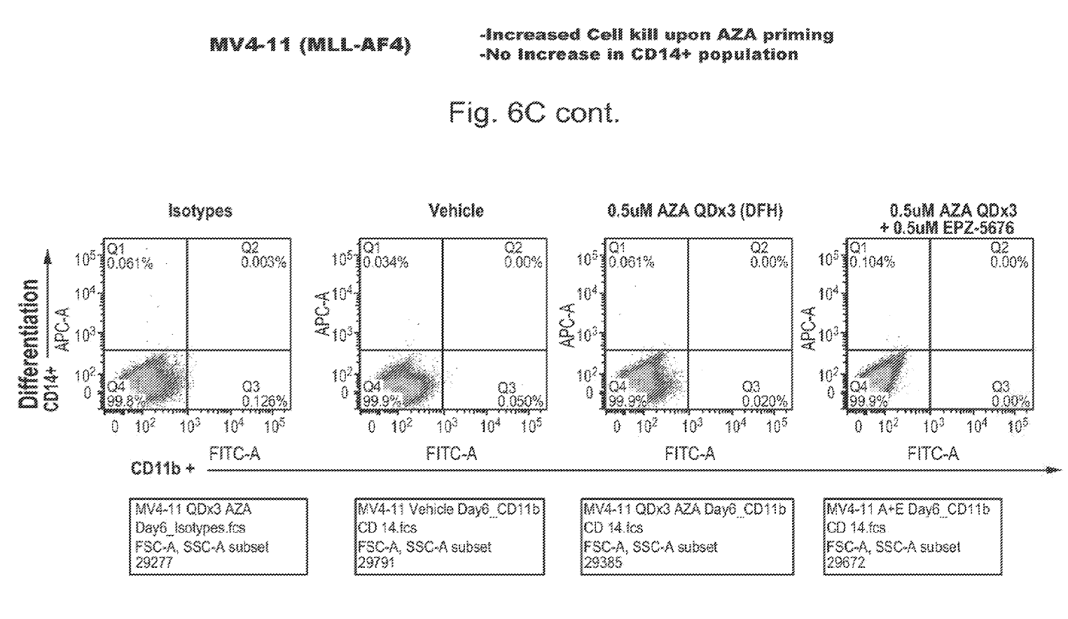

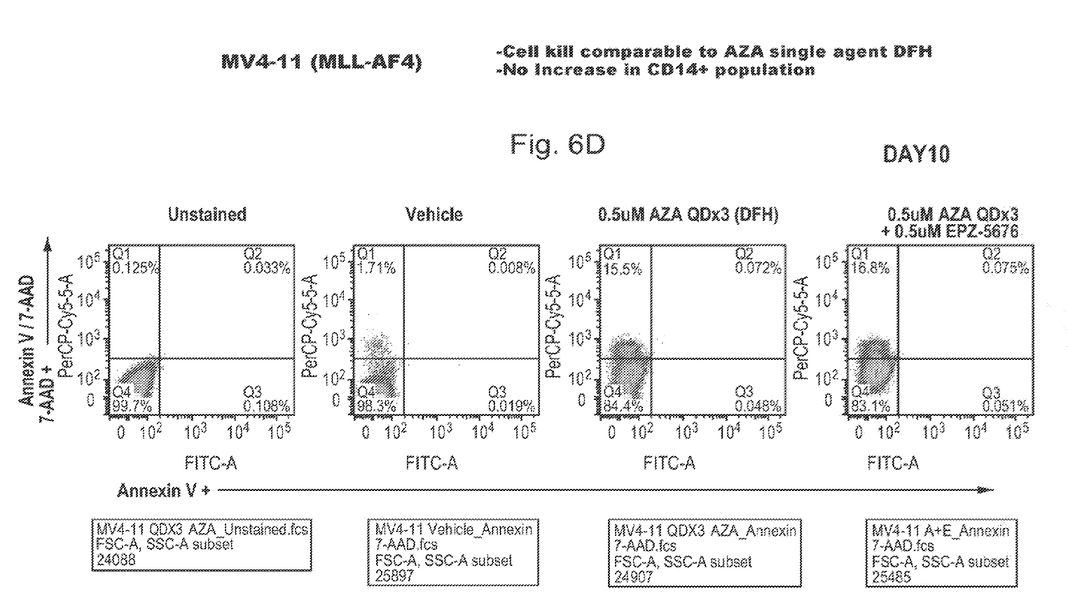

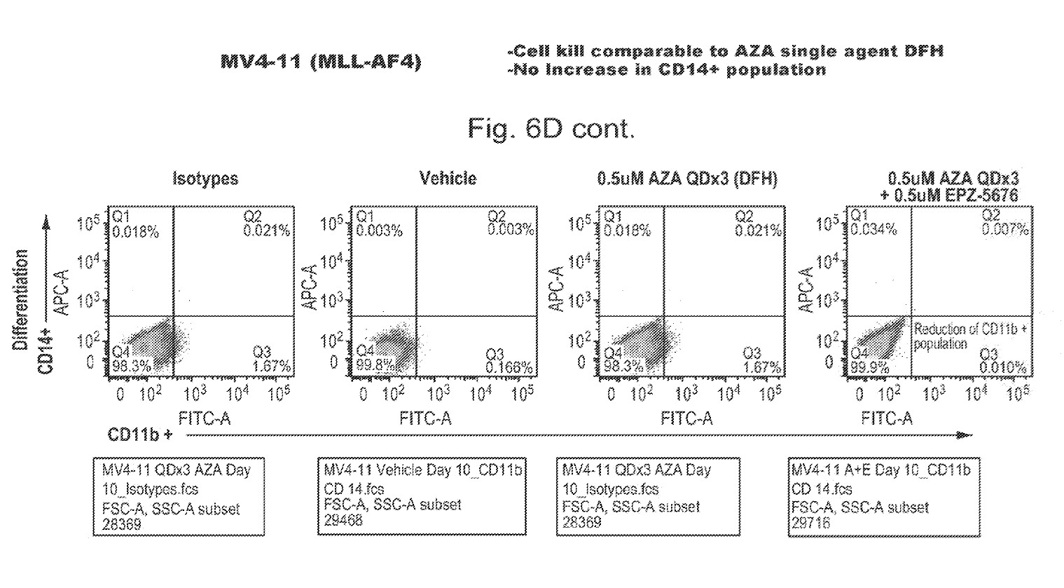

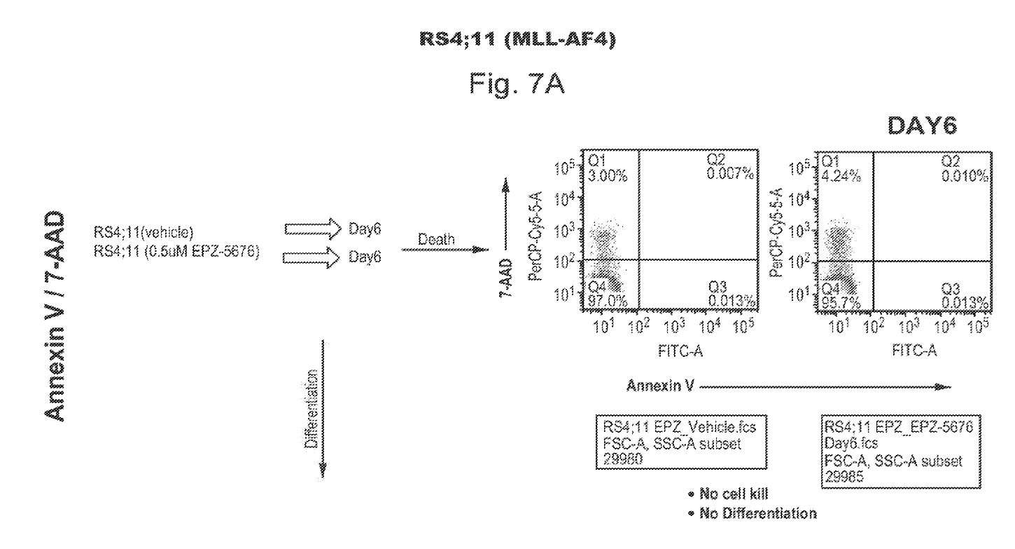

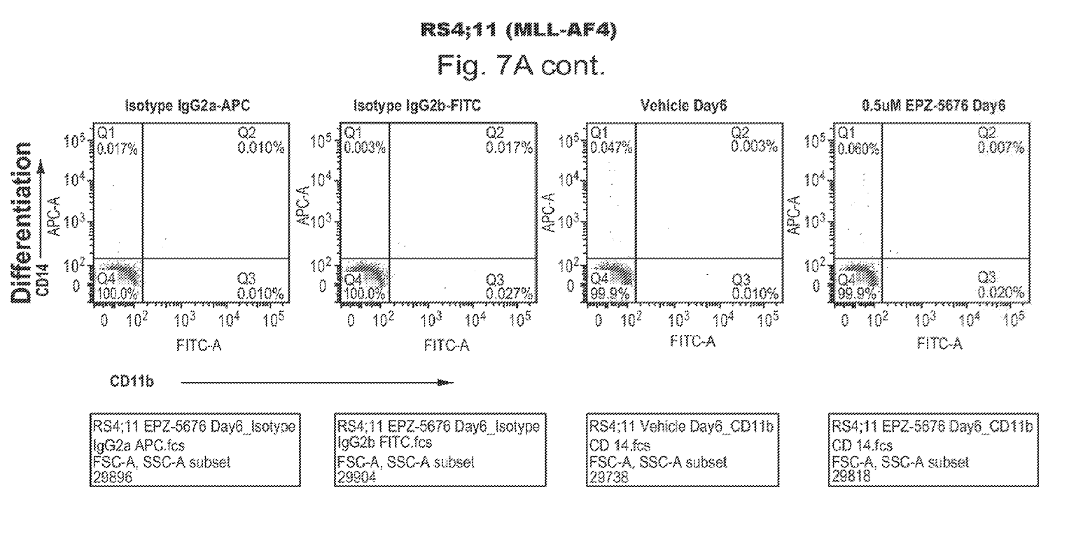

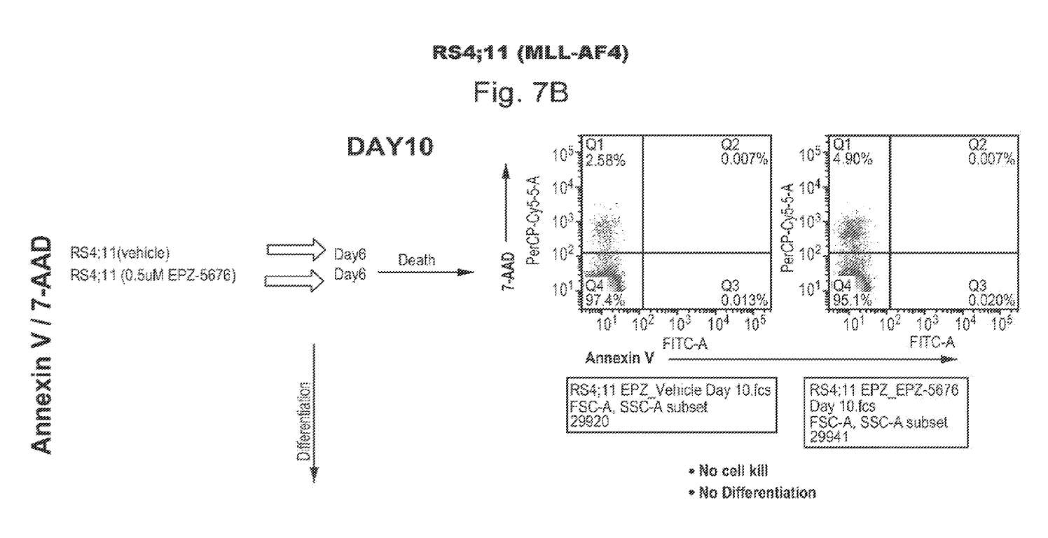

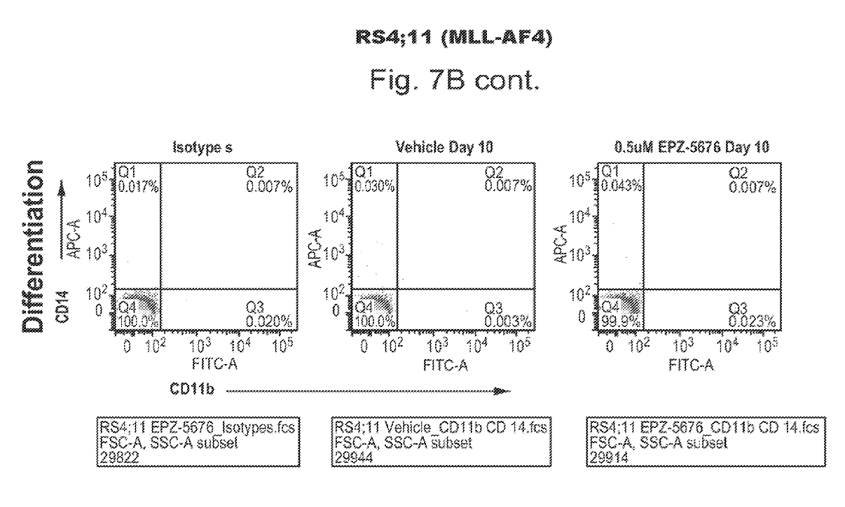

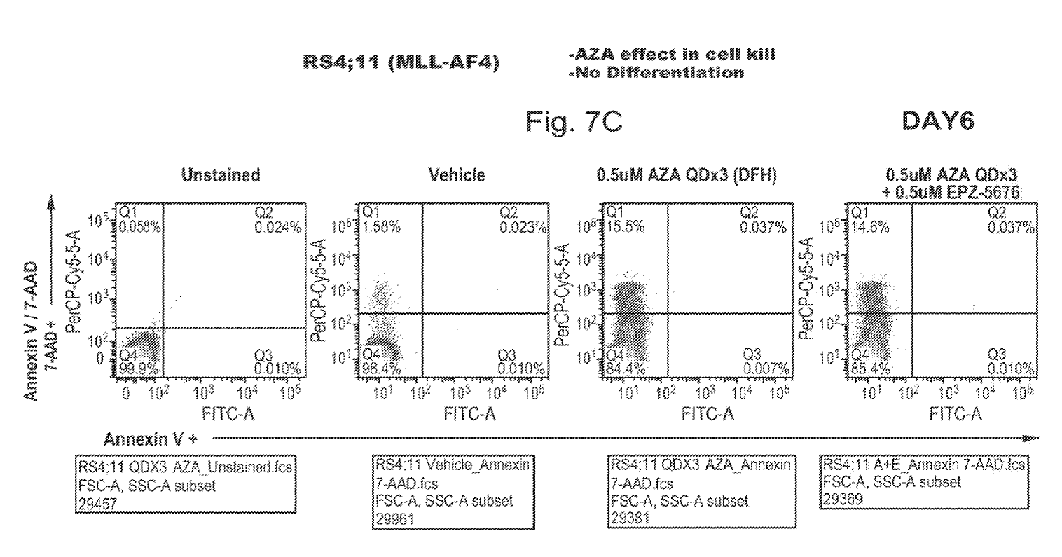

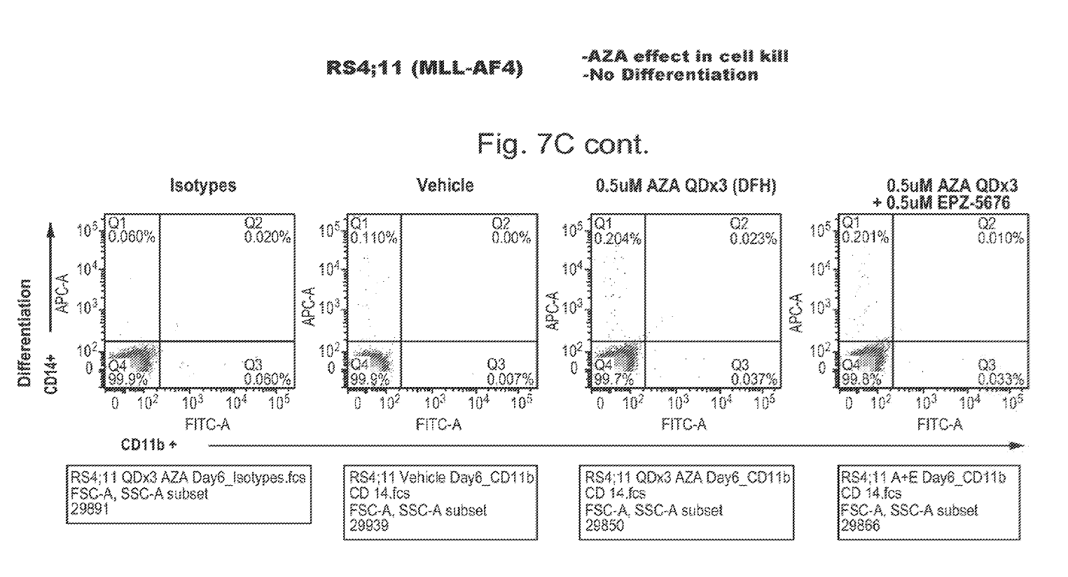

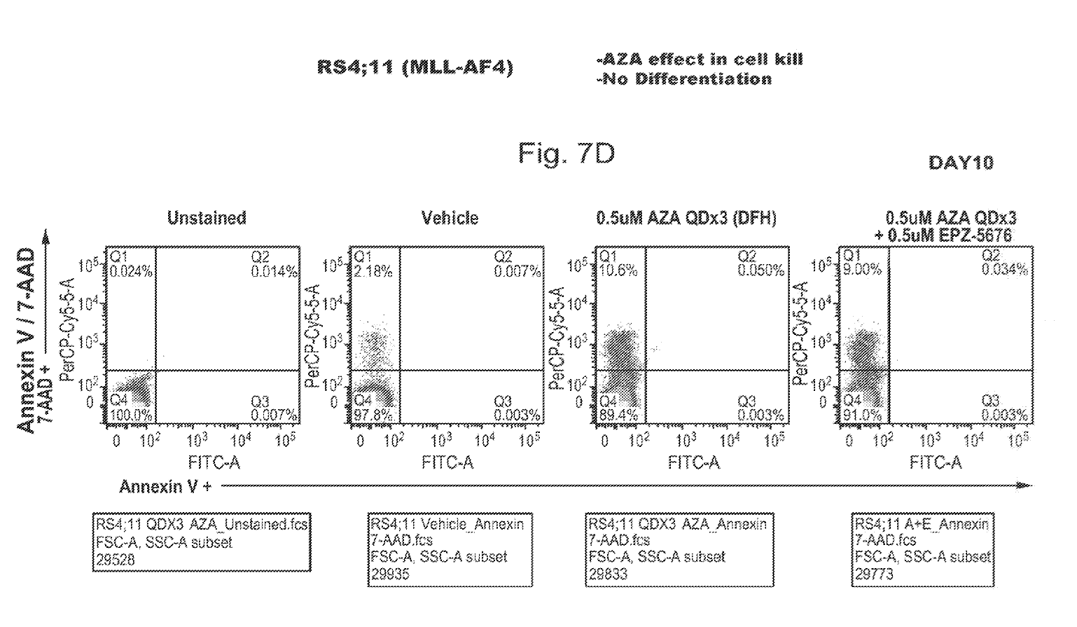

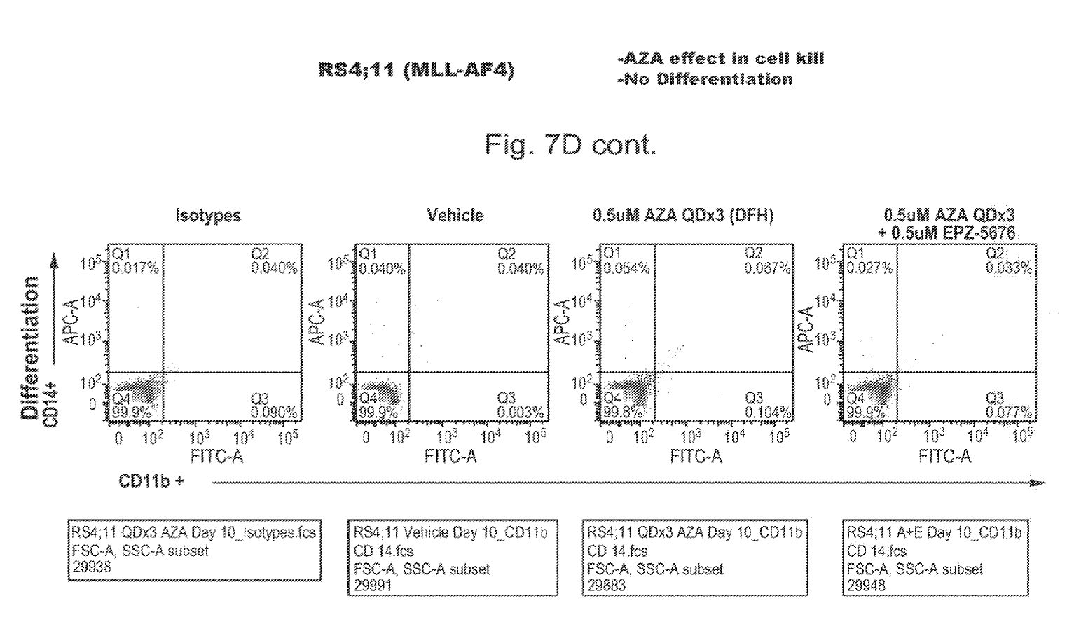

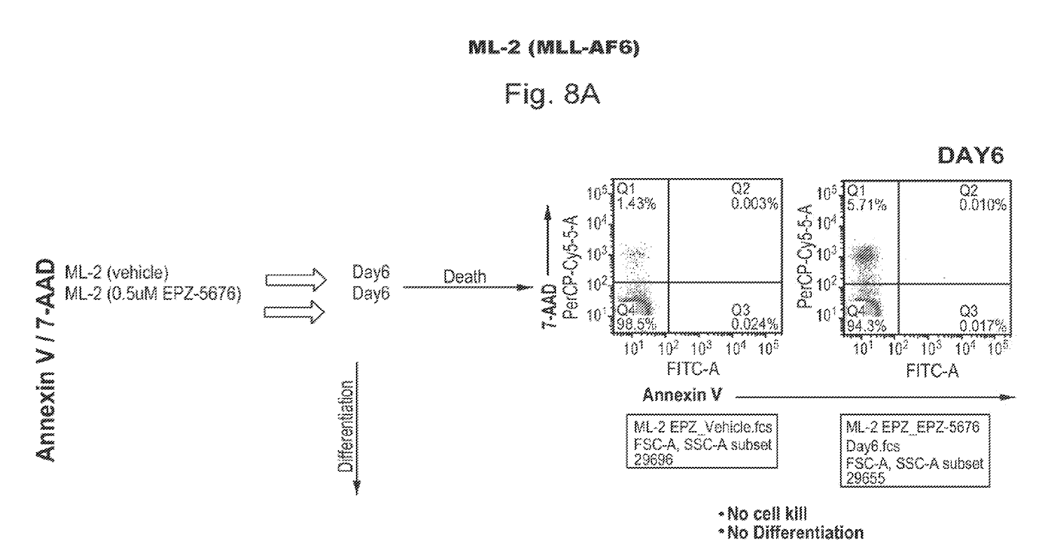

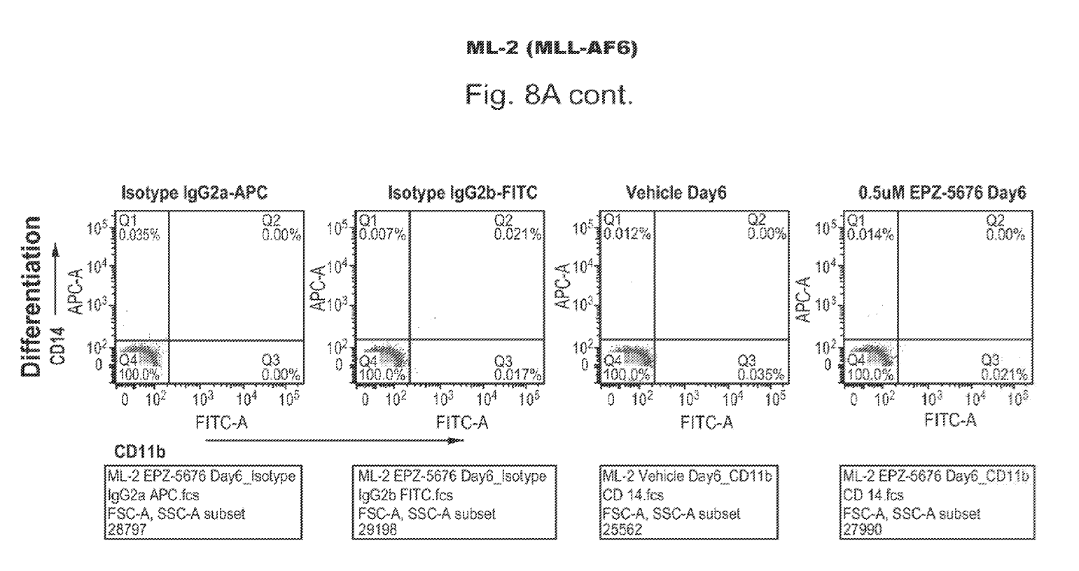

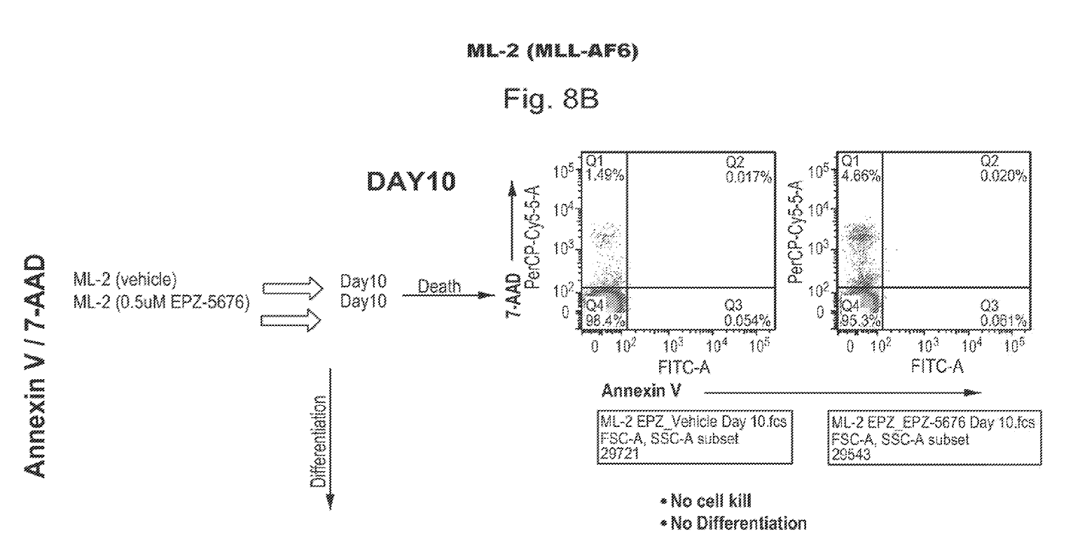

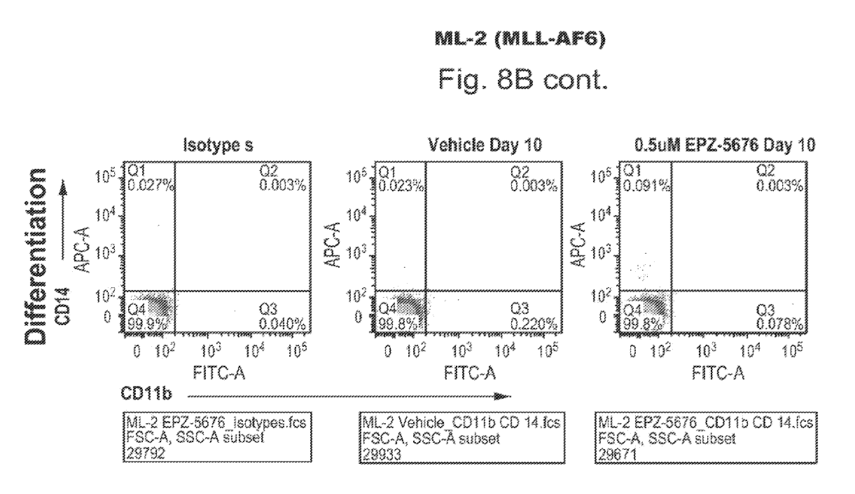

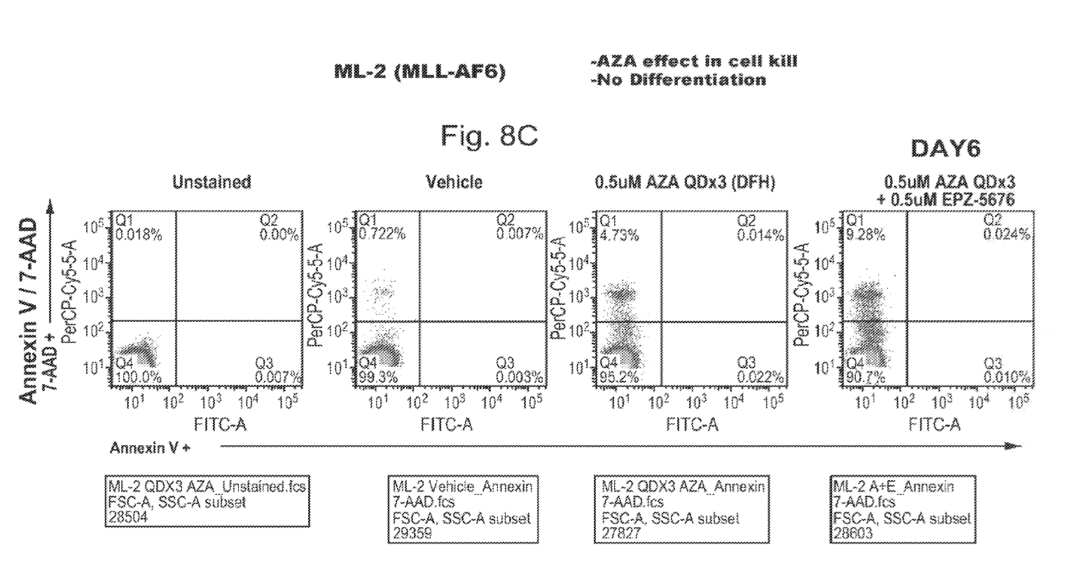

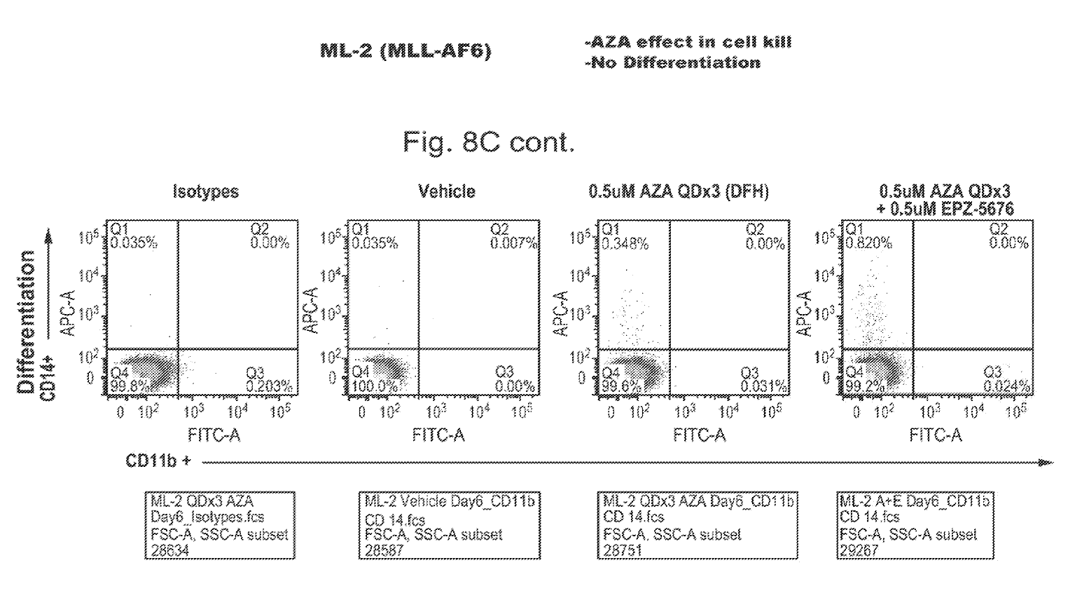

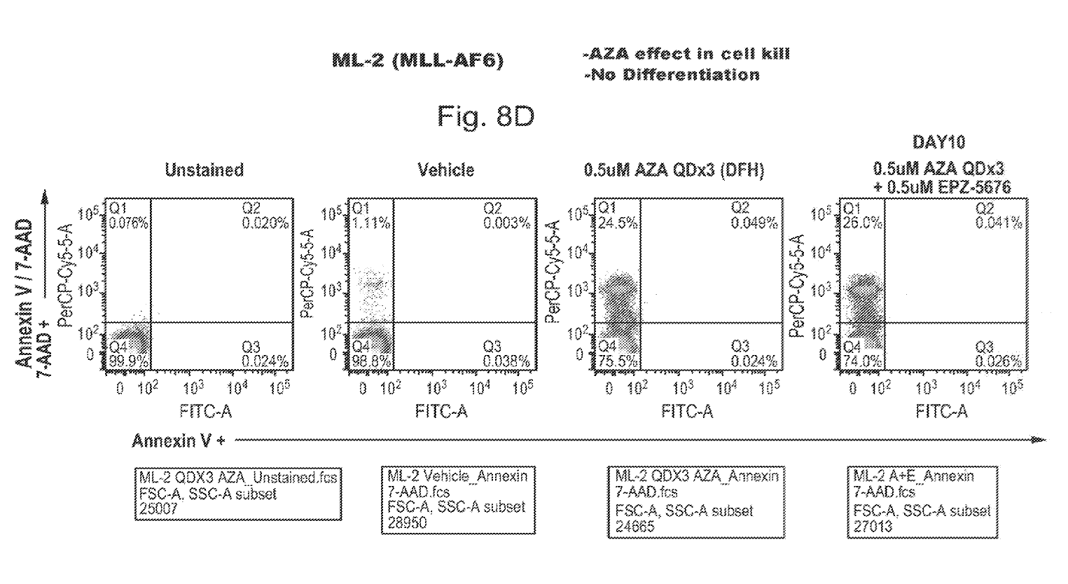

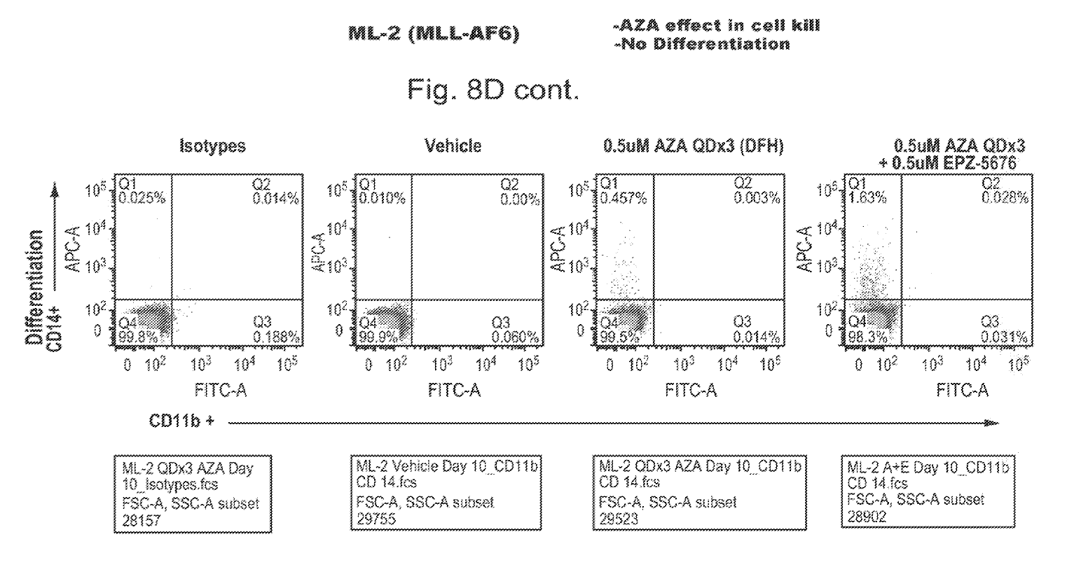

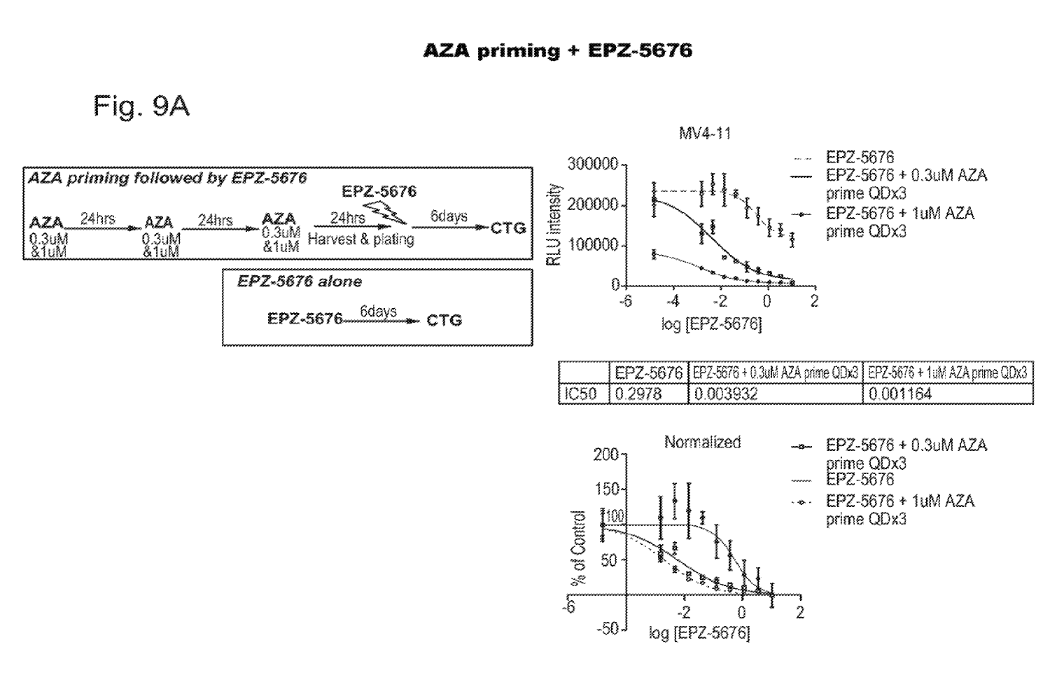

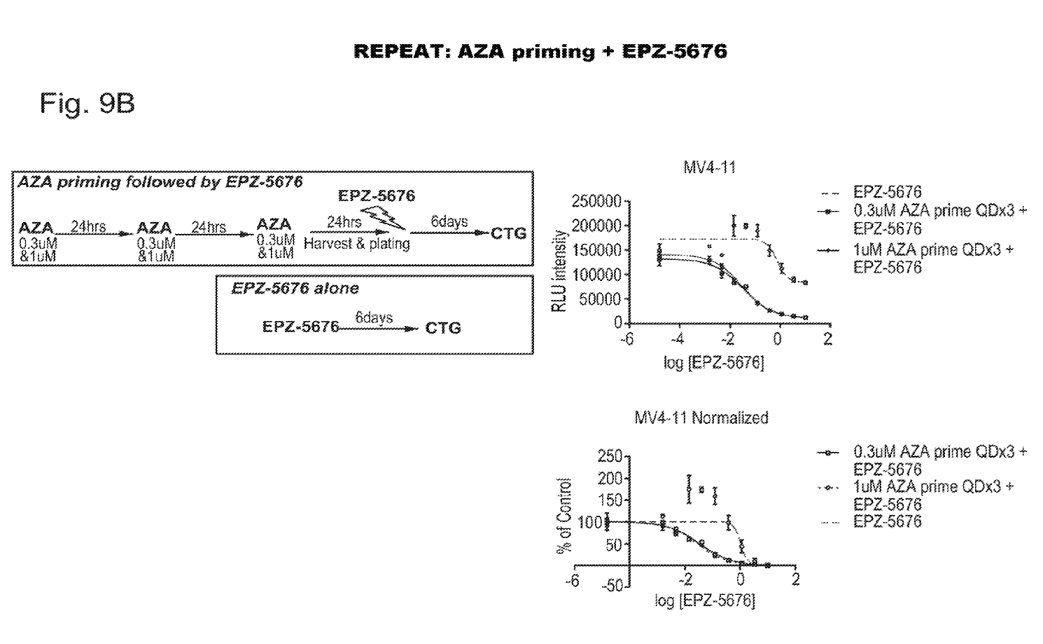

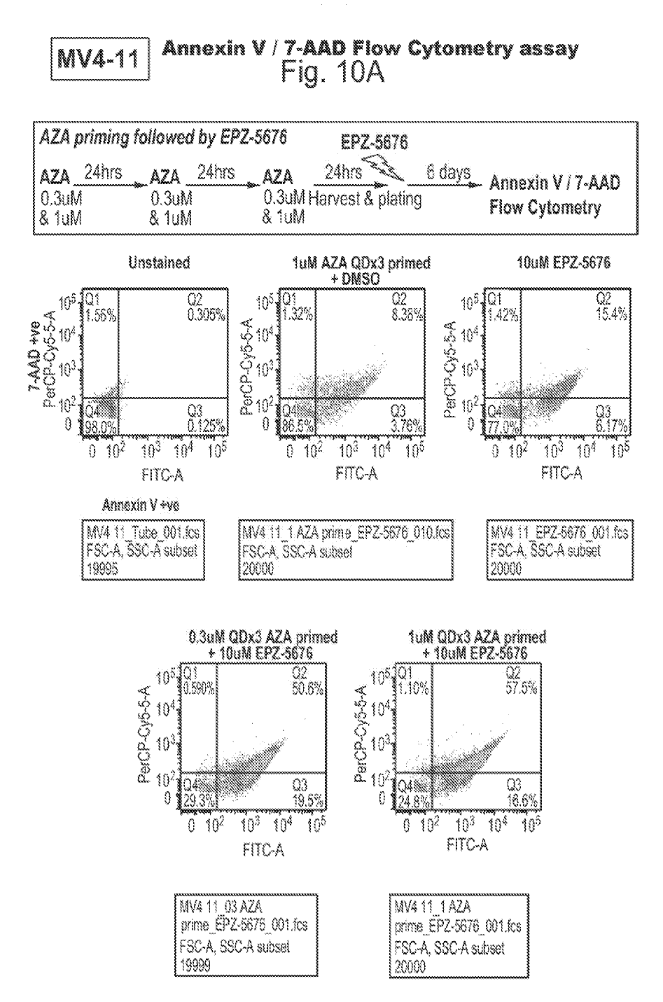

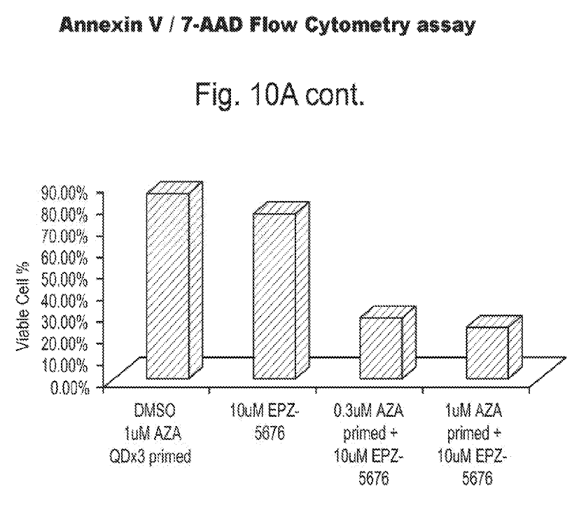

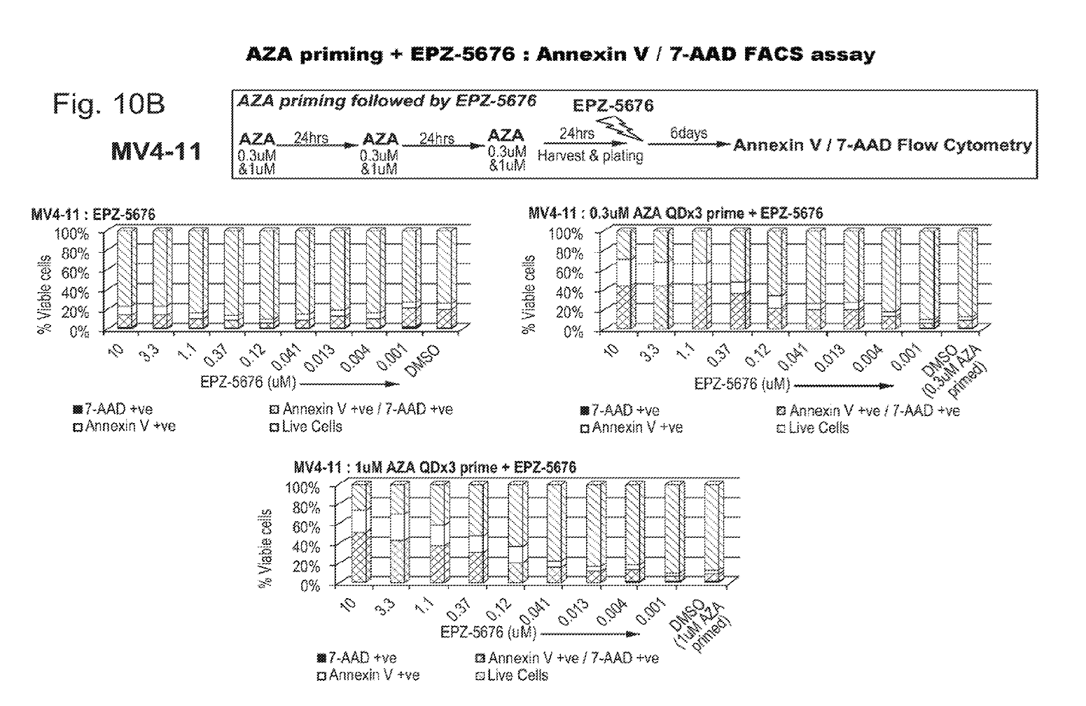

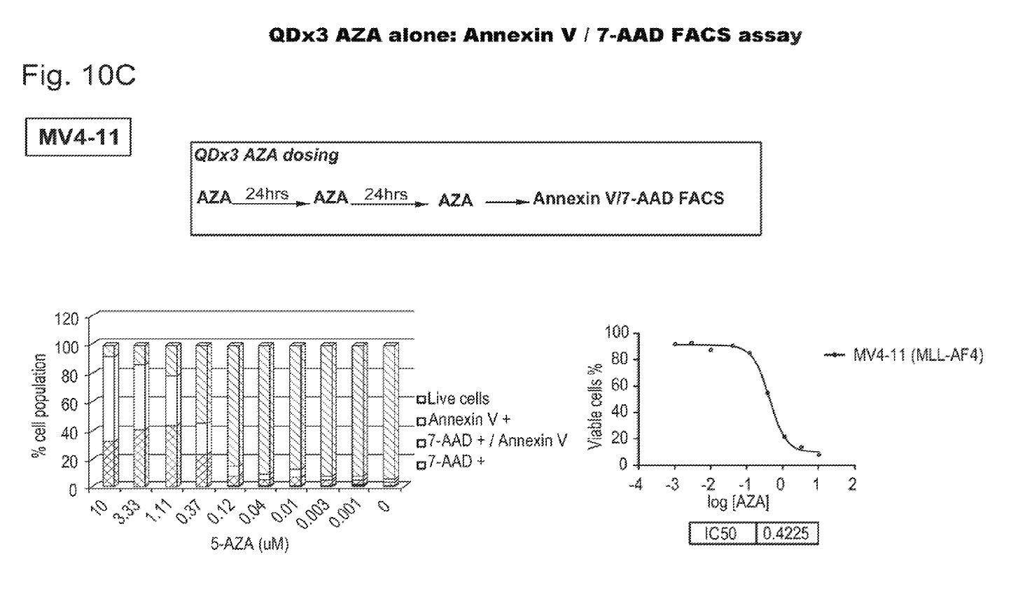

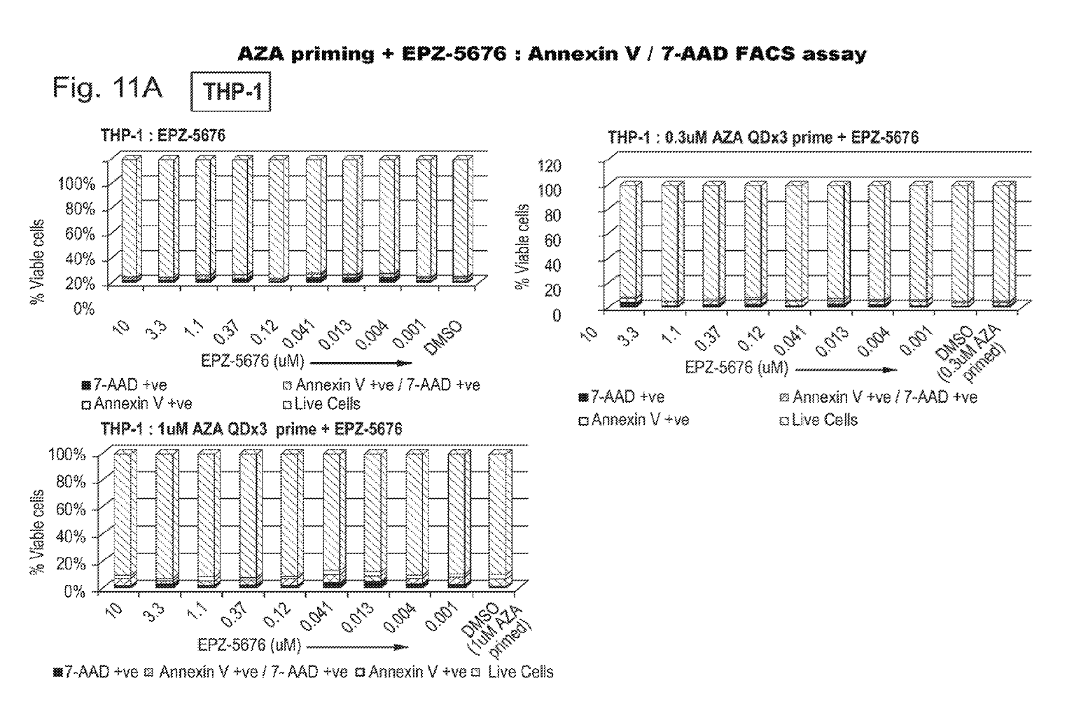

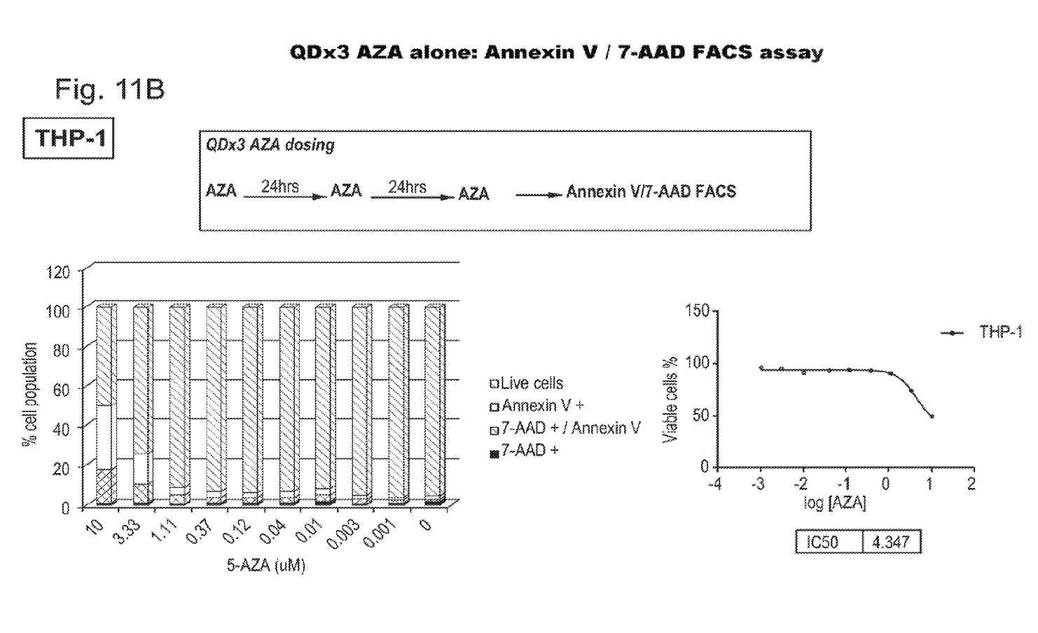

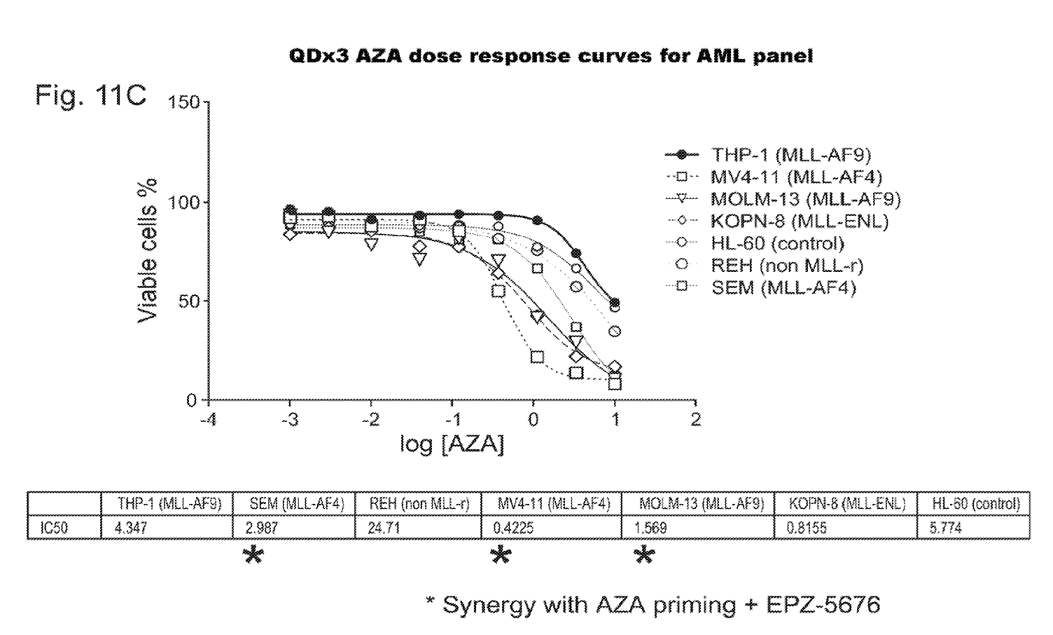

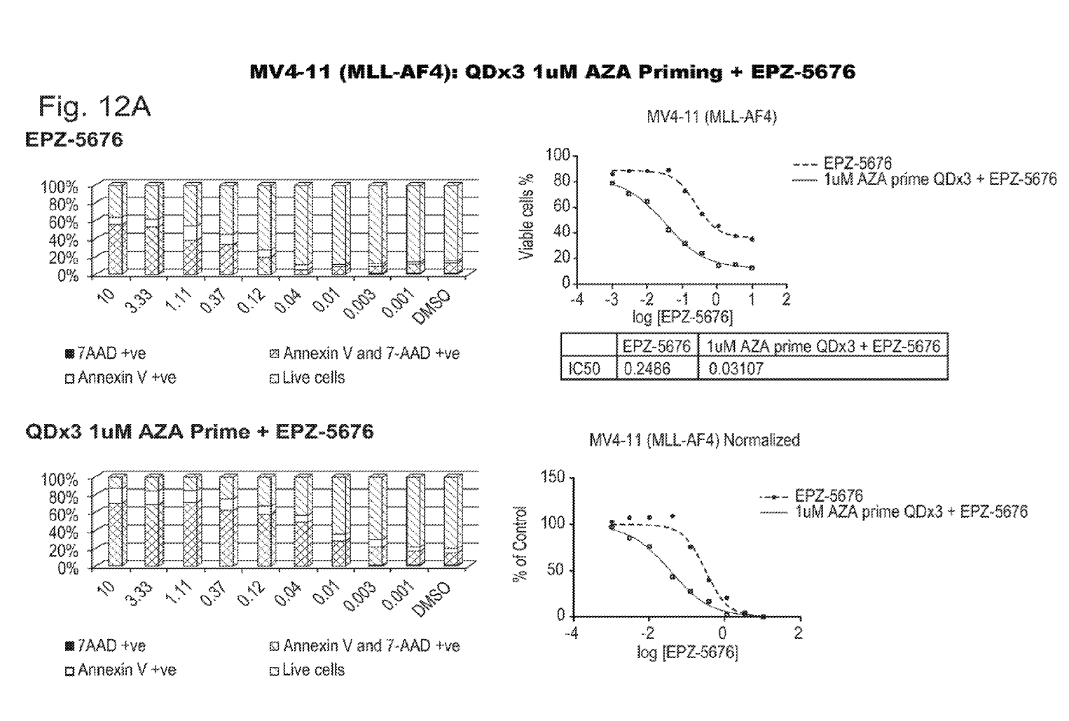

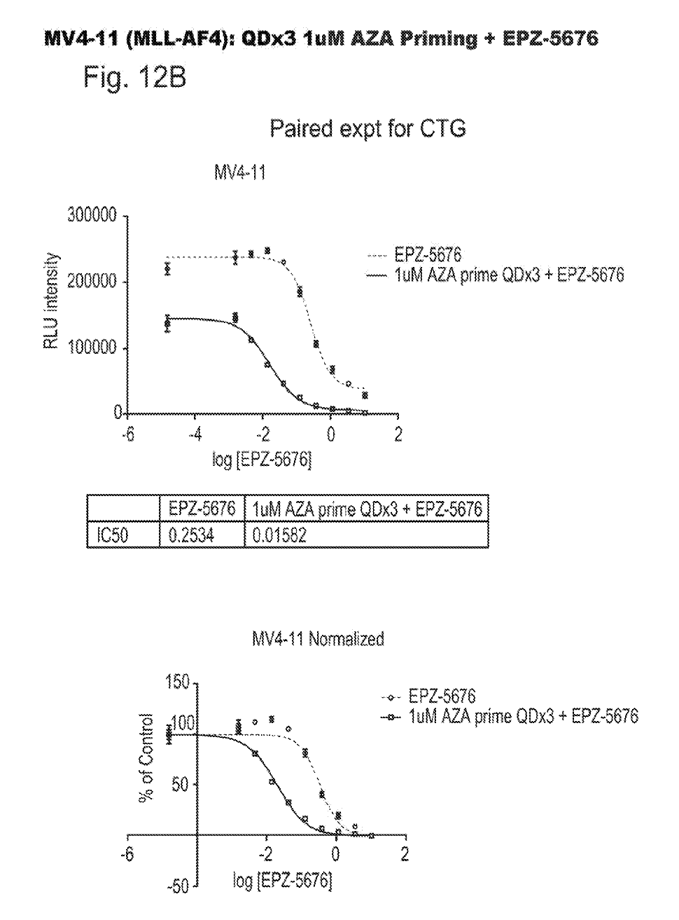

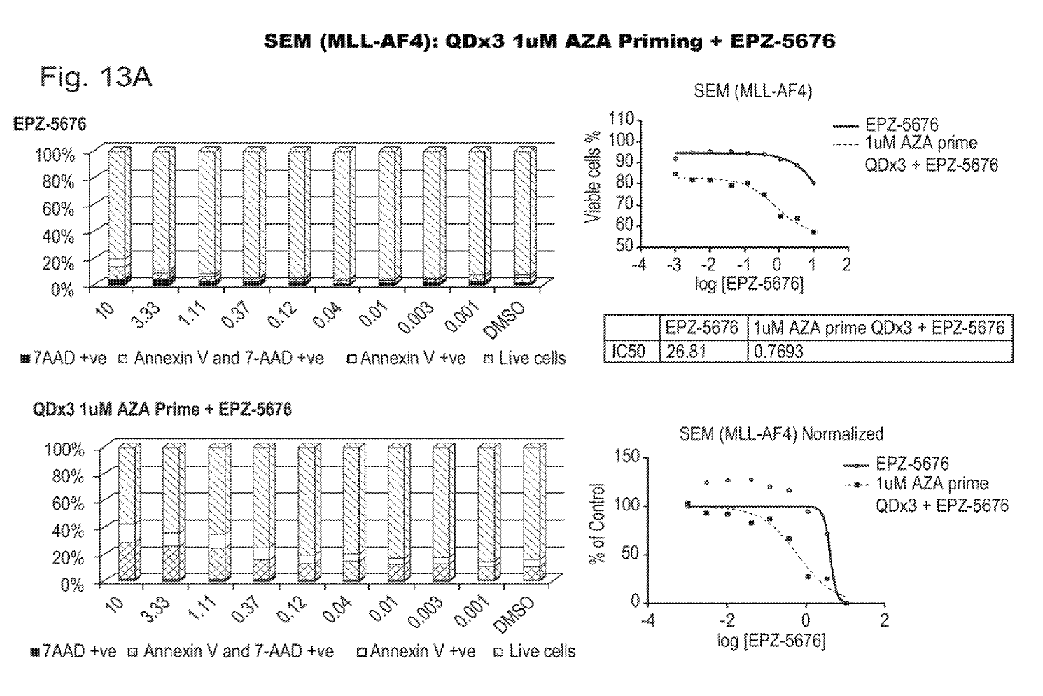

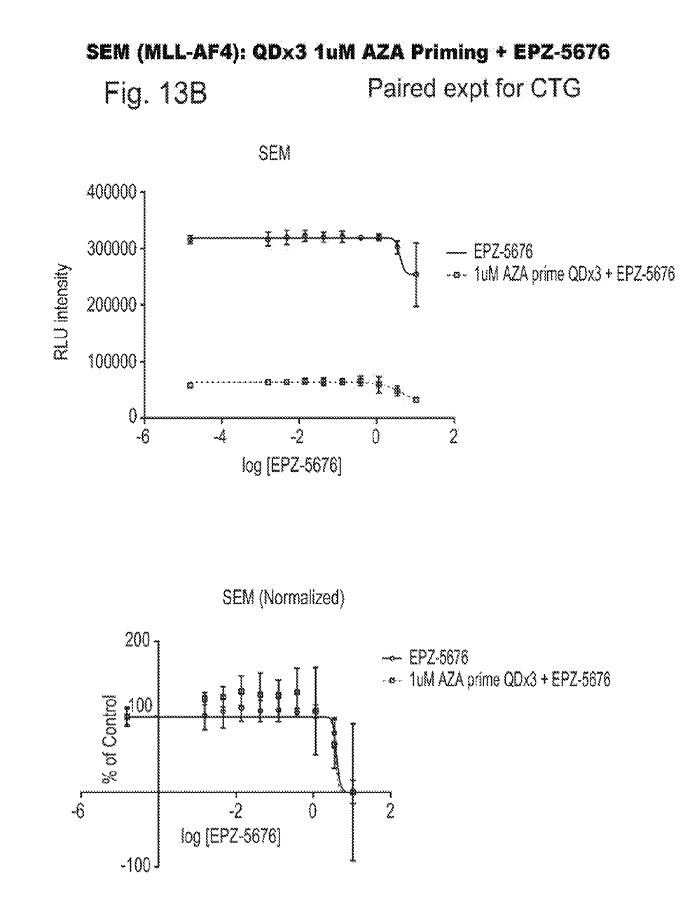

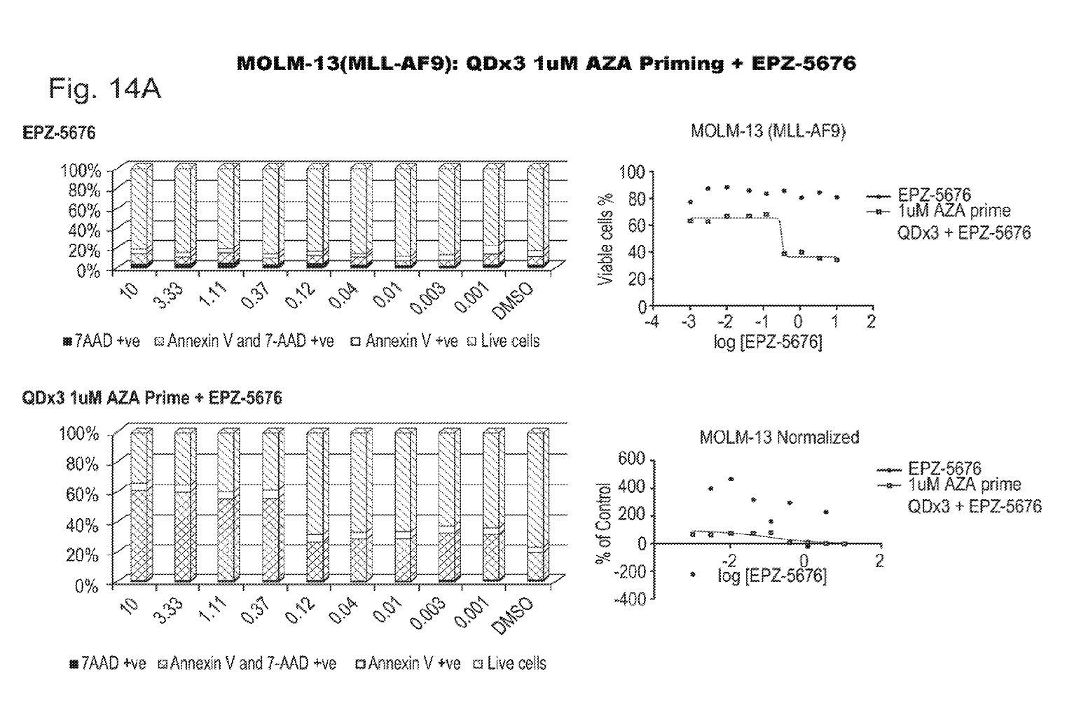

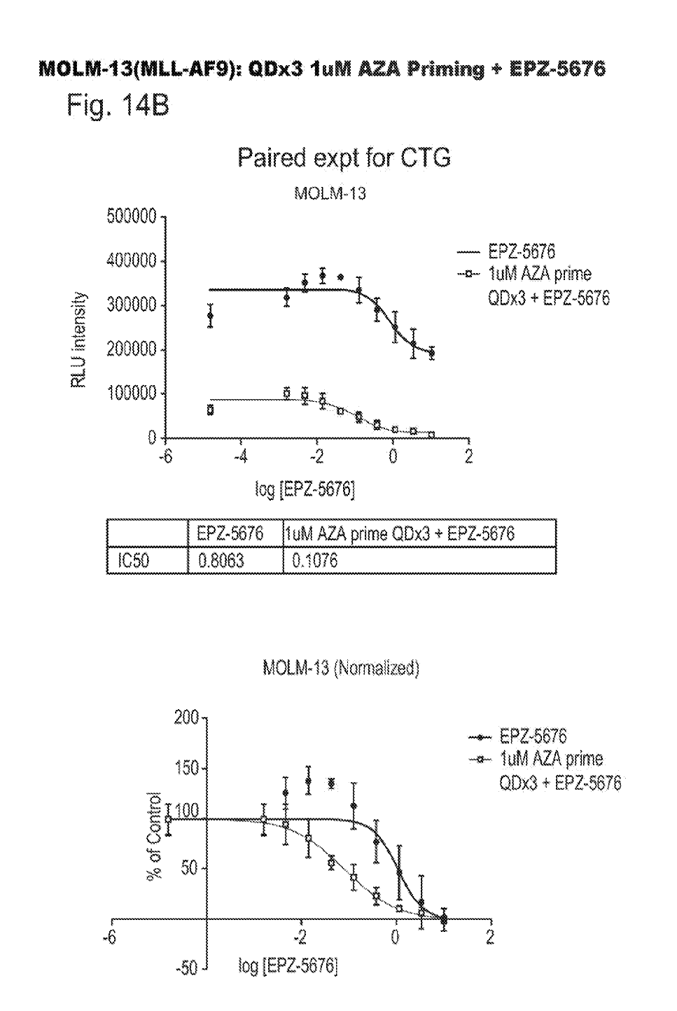

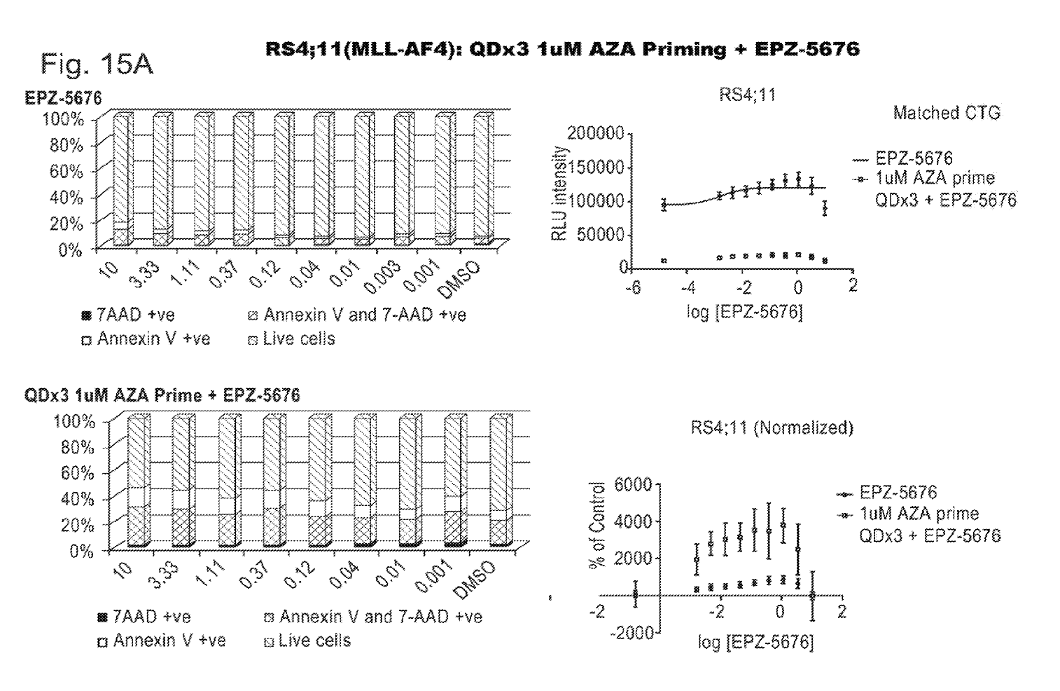

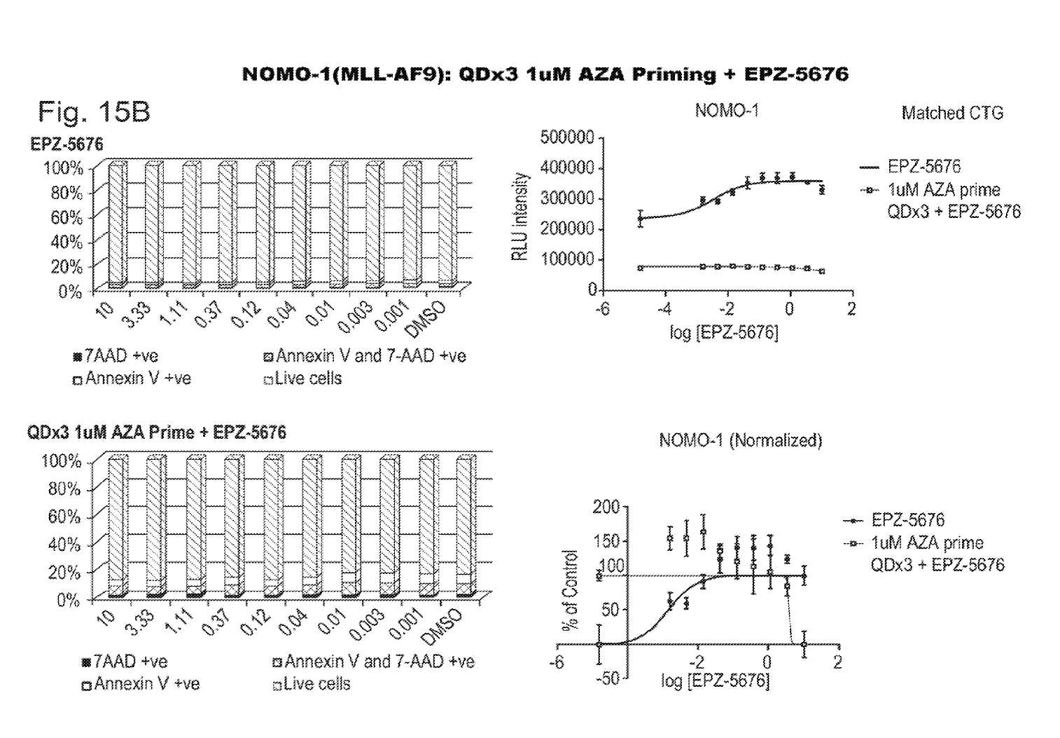

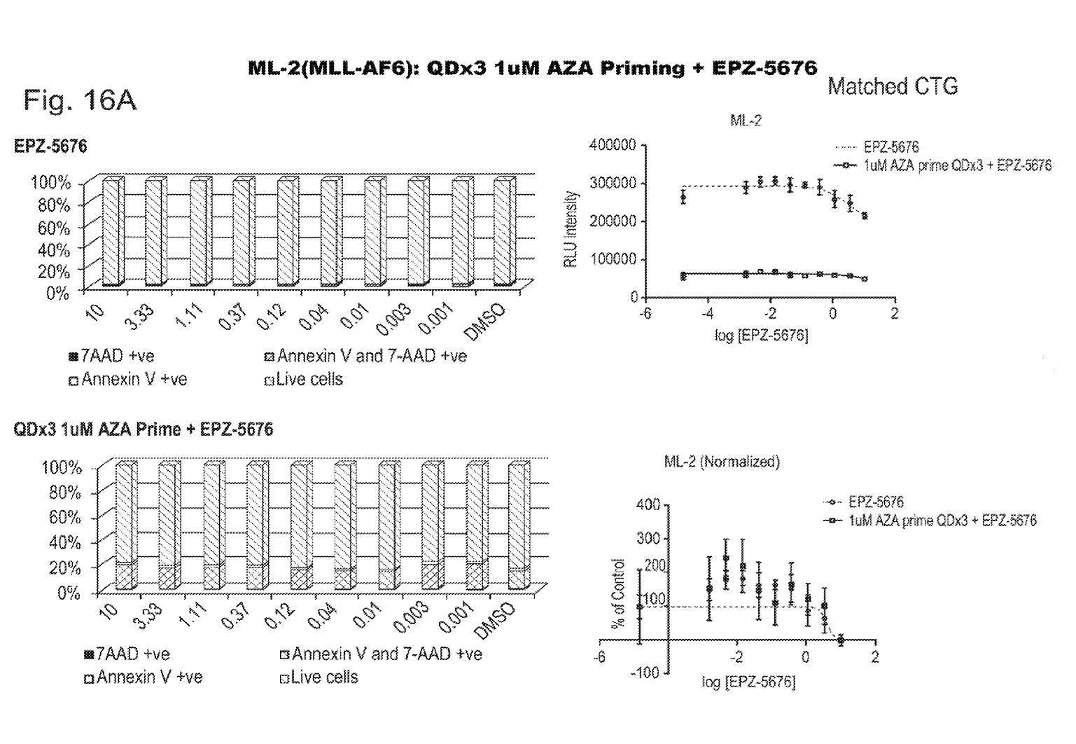

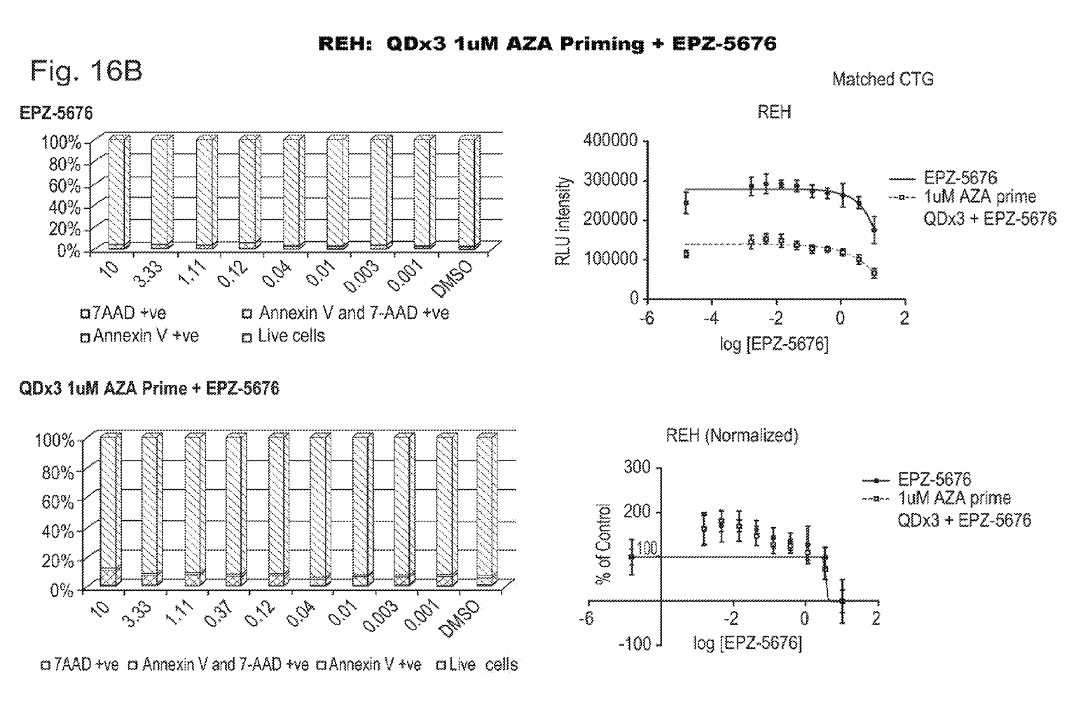

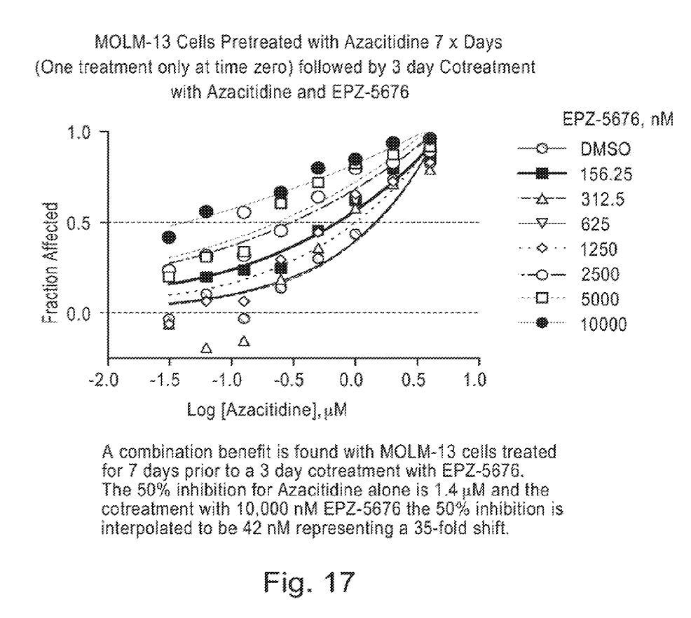

EPZ-5676 is a small molecule inhibitor of the histone methyltransferase DOT1L currently in clinical development and representing a new class of therapeutic agents for the treatment of MLL-rearranged (MLL-r) leukemia. Preclinically, EPZ-5676 selectively inhibits intracellular histone H3K79 methylation, downstream target gene expression and demonstrated complete tumor regressions in an MLL rearranged leukemia xenograft model. Synergistic and durable anti-proliferative activity was previously reported when EPZ-5676 was combined with current AML standard of care (SOC) drugs in MLL-r leukemia models MOLM-13 (MLL-AF9) and MV4-11 (MLL-AF4). See, e.g., WO 2014/153001, the content of which is hereby incorporated by reference in its entirety. Combination benefit was also observed when MLL-r cells were treated with Cytarabine prior to co-treatment with EPZ-5676. See, e.g., co-pending applications 62/051,890 filed on Sep. 17, 2014, 62/088,498 filed on Dec. 5, 2014, and PCT/US2014/065517 filed on Nov. 13, 2014, and WO 2014/153001, the contents of each of which are hereby incorporated by reference in their entireties. Additionally, both Cytarabine and Azacitidine, a DNA methyltransferase inhibitor displayed synergistic antileukemic activity in MLL-r rearranged cells in a 7 day co-treatment model, in which the cells were treated for 7 days with EPZ-5676 and then the second agent (Klaus et al., J Pharmacol Exp Ther. 2014; 350(3):646-56.). In this study of Example 1, the flexibility of treatment schedules when EPZ-5676 was combined with Azacitidine in MLL-r cells was evaluated. Cells were pretreated with Azacitidine at nanomolar concentrations identified to reverse promoter DNA-hypermethylation and alter the chromatin state (Tsai H C Cancer Cell. 2012 Mar. 20; 21(3):430-46). It was found that one time daily treatment for three consecutive days with Azacitidine of MV4-11 and MOLM-13 cell lines followed by sequential treatment with EPZ-5676 elicited a synergistic antiproliferative effect. The drug combination analysis was performed using the Chou-Talalay method (Chou T C. Pharmacol Rev. 2006 September; 58(3):621-81). Mechanistic studies to investigate the mechanism of synergistic cell killing, e.g. evaluation of the expression of markers of differentiation and Annexin V staining are under way. See FIGS. 1-19.

These data indicate that AZA priming followed by EPZ-5676 treatment has a synergistic effect that results in cell death in three out of 10 AML cell lines. The following AML cell lines display synergy for cell kill upon AZA priming (6 days): MV4-11 (MLL-AF4), MOLM-13 (MLL-AF9), and SEM (MLL-AF4). The following cell lines are resistant to EPZ-5676 and do not show synergy upon AZA priming (6 days): REH (non-rearranged control), RS4;11 (MLL-AF4), NOMO-1 (MLL-AF9), ML-2 (MLL-AF6).

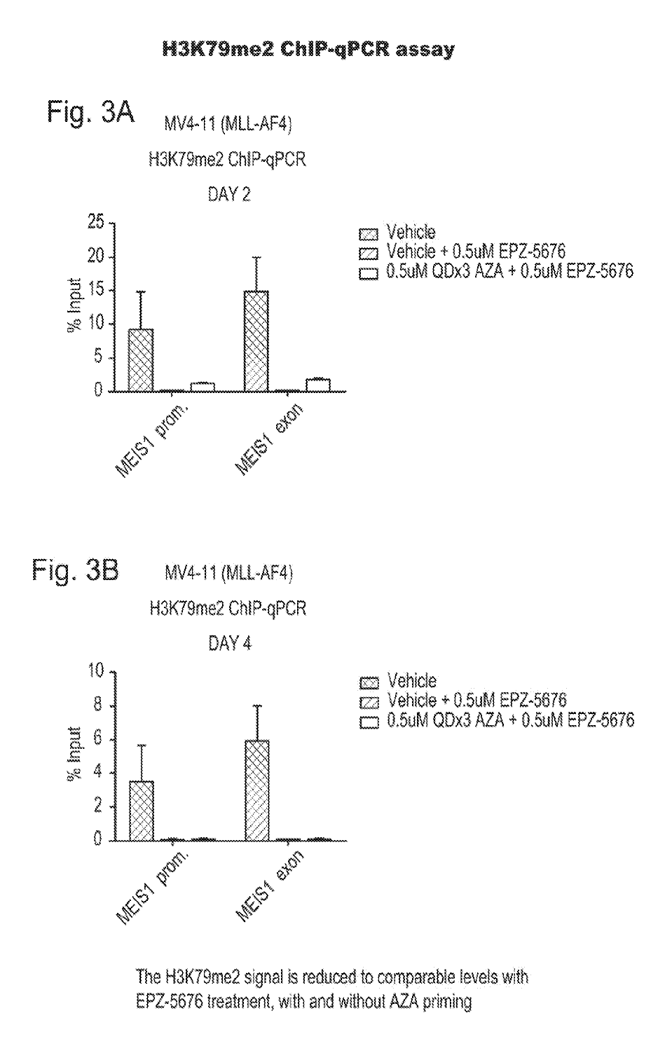

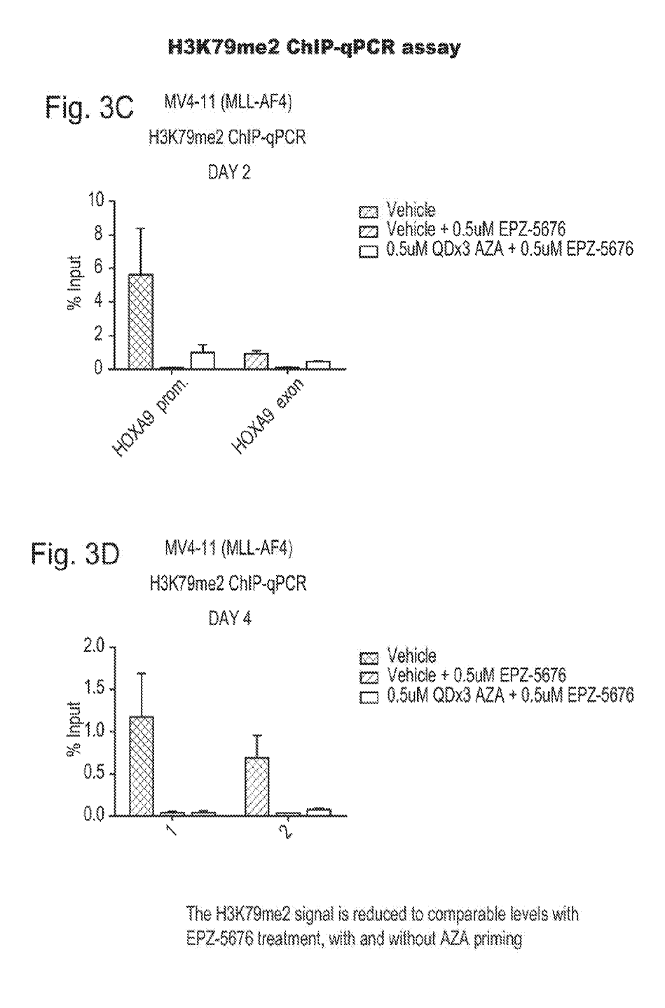

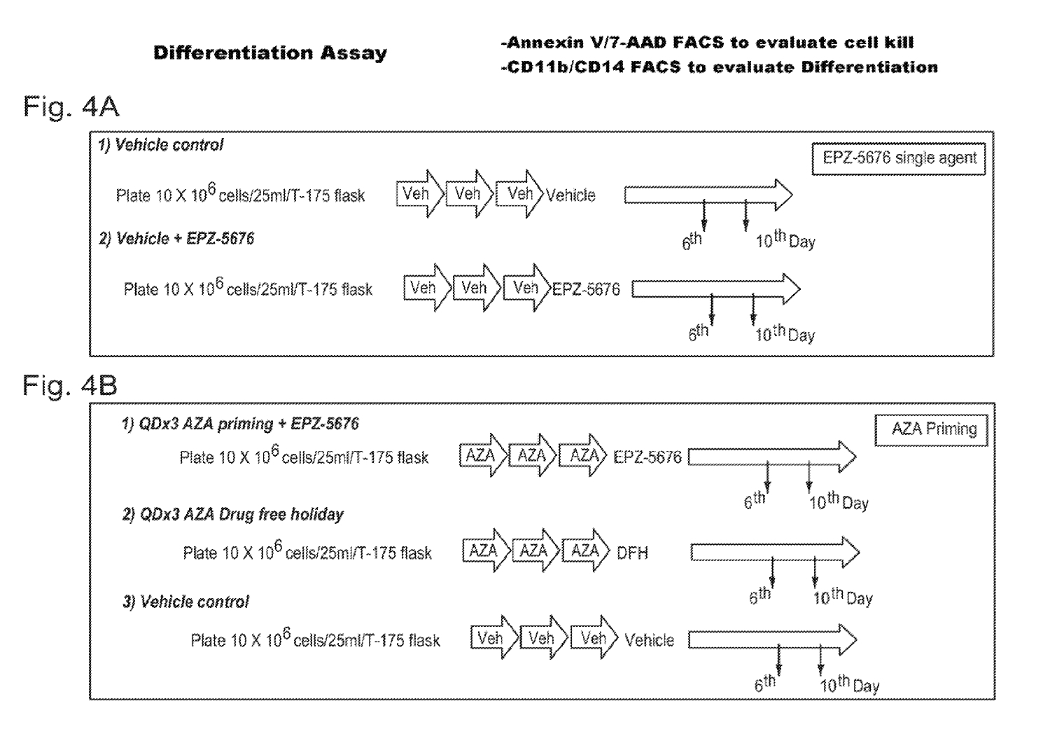

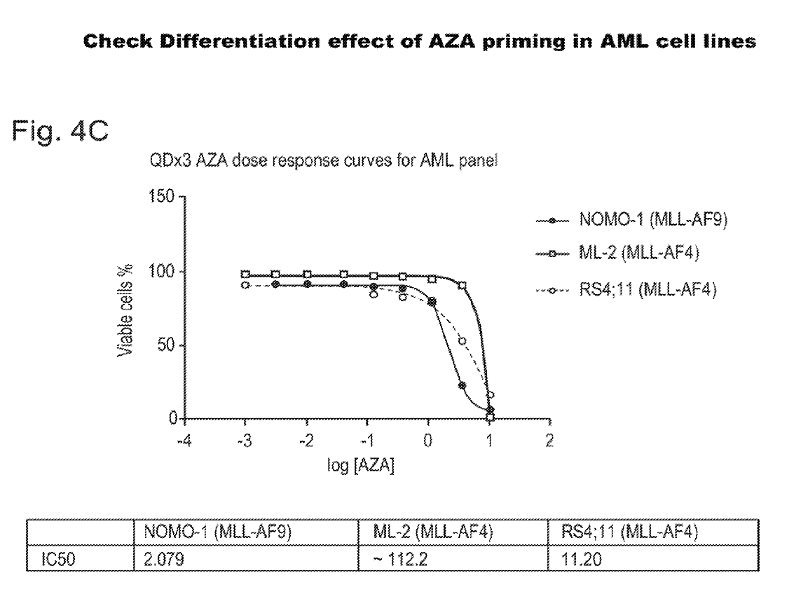

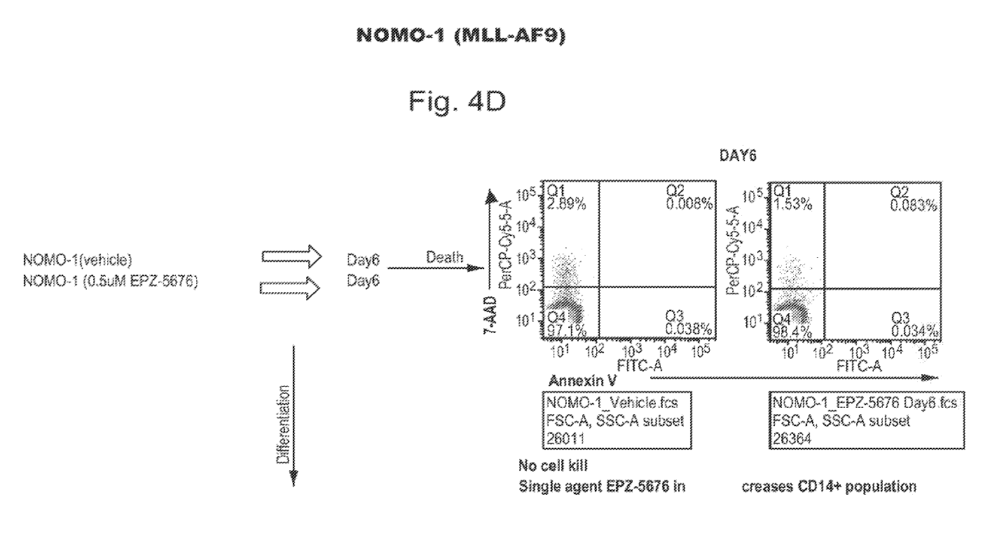

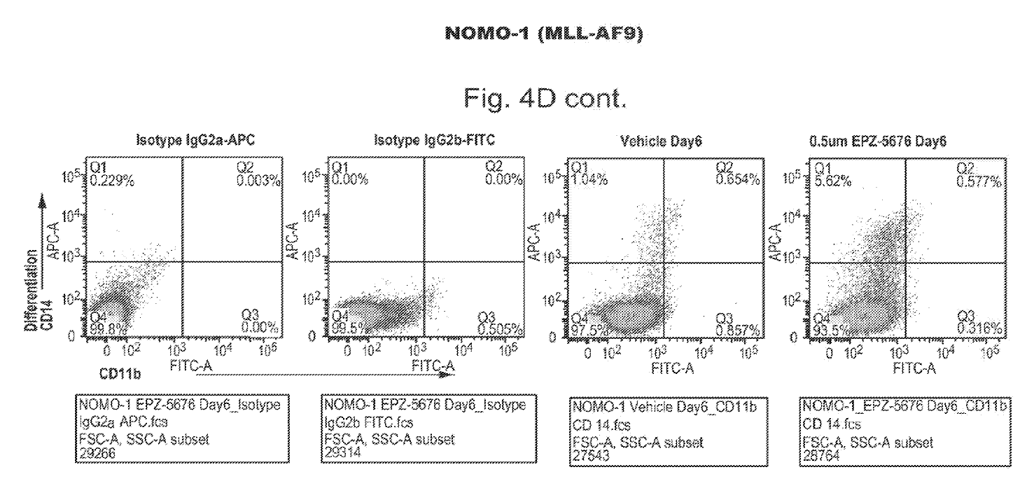

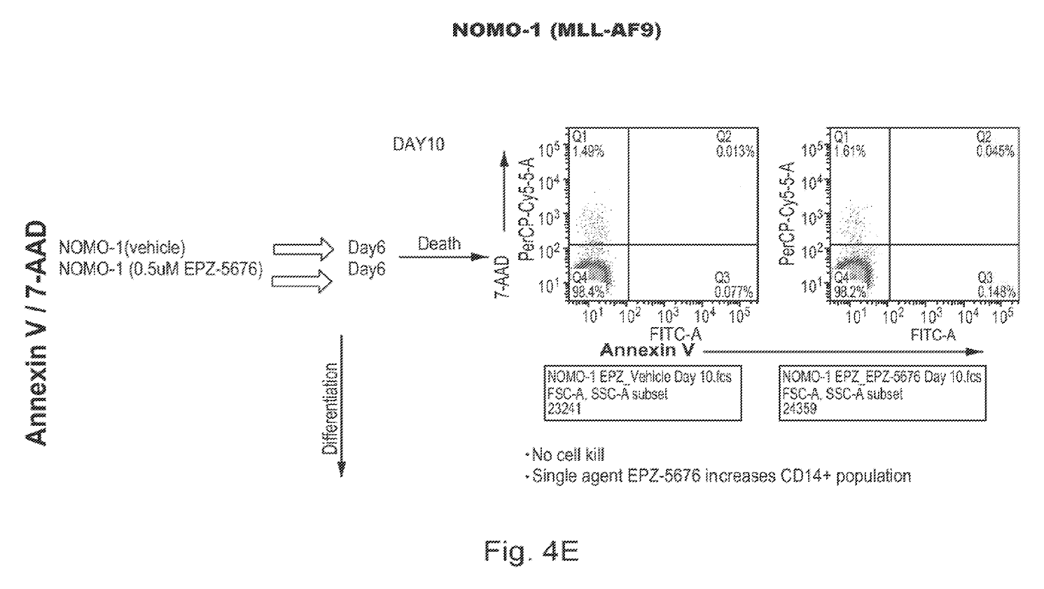

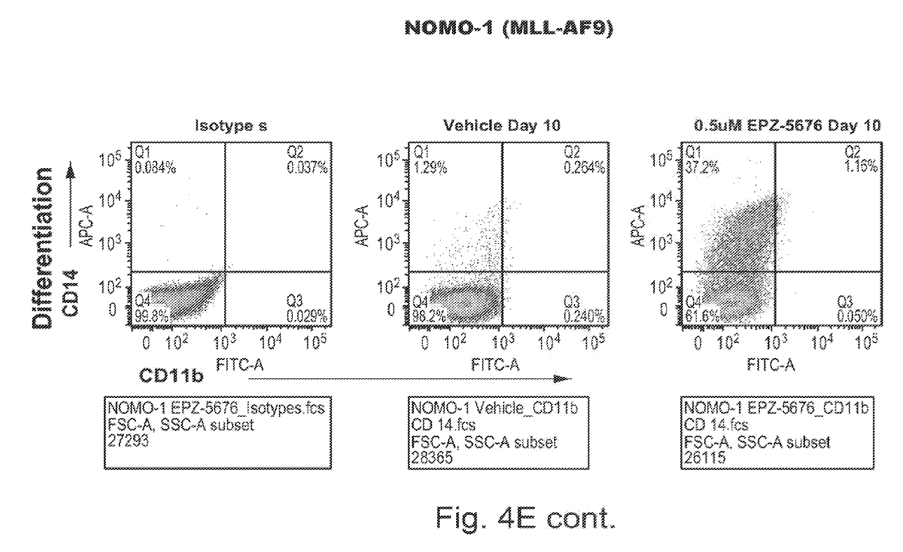

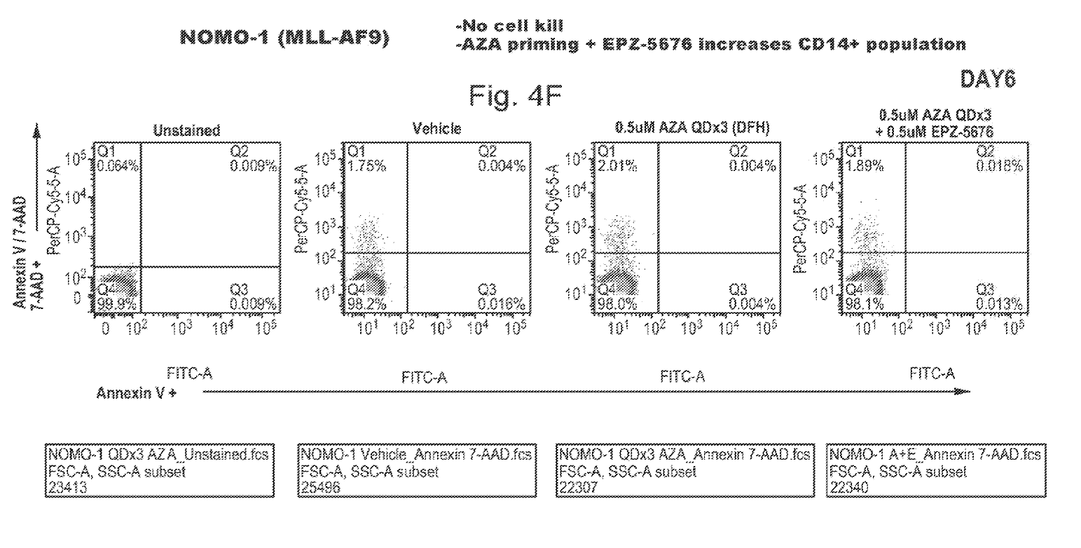

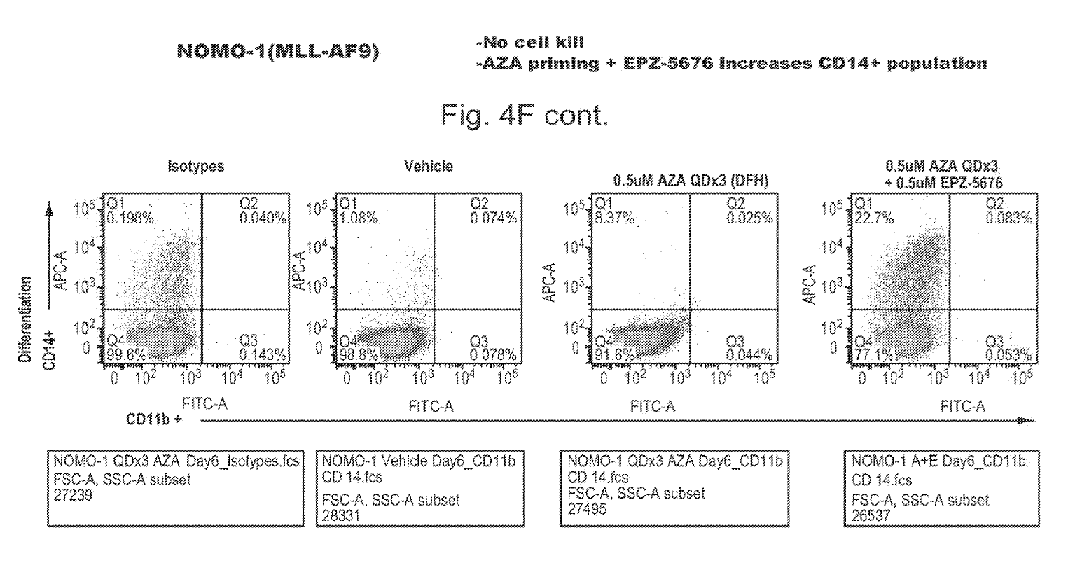

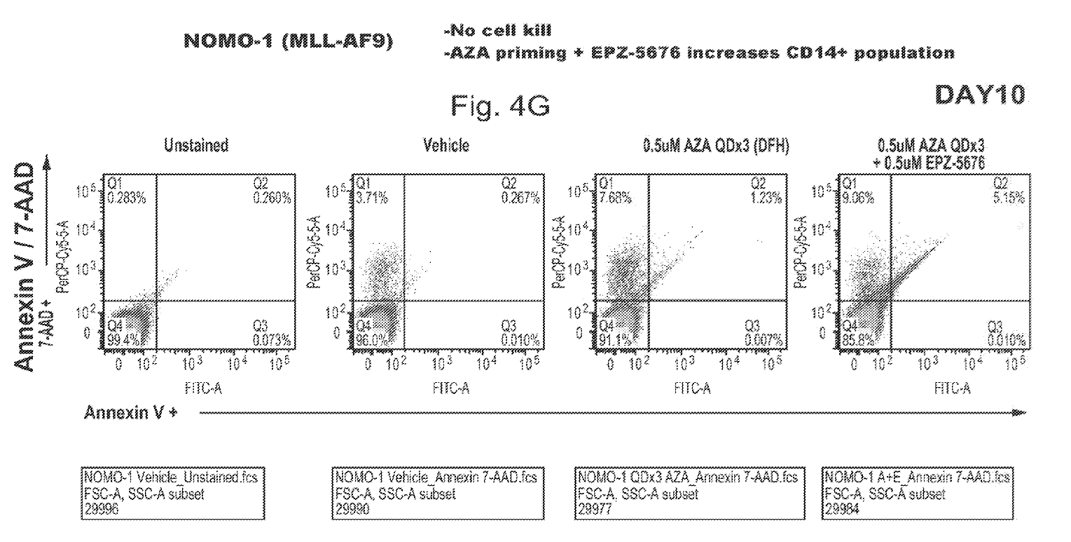

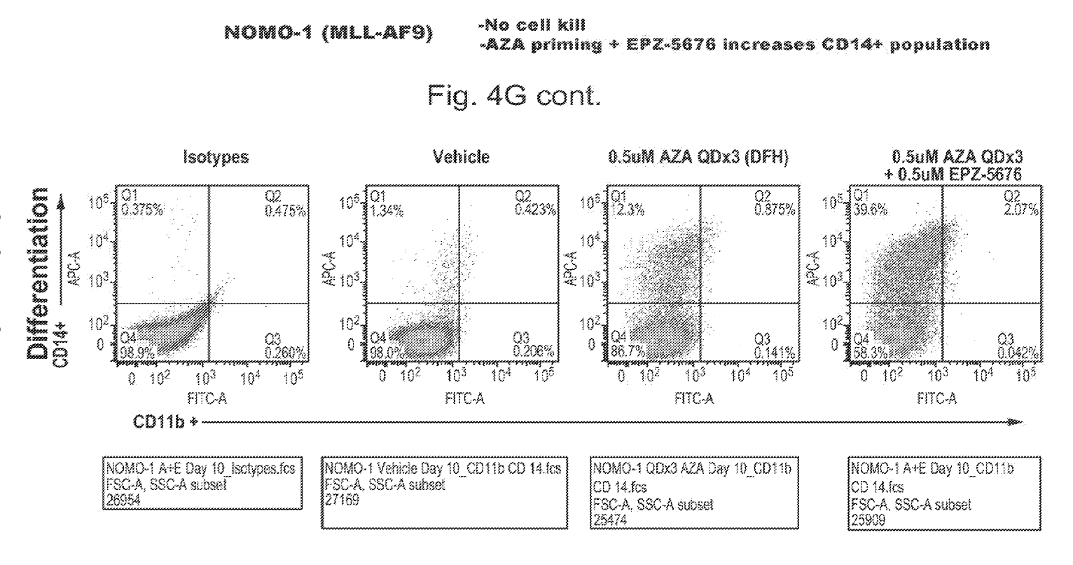

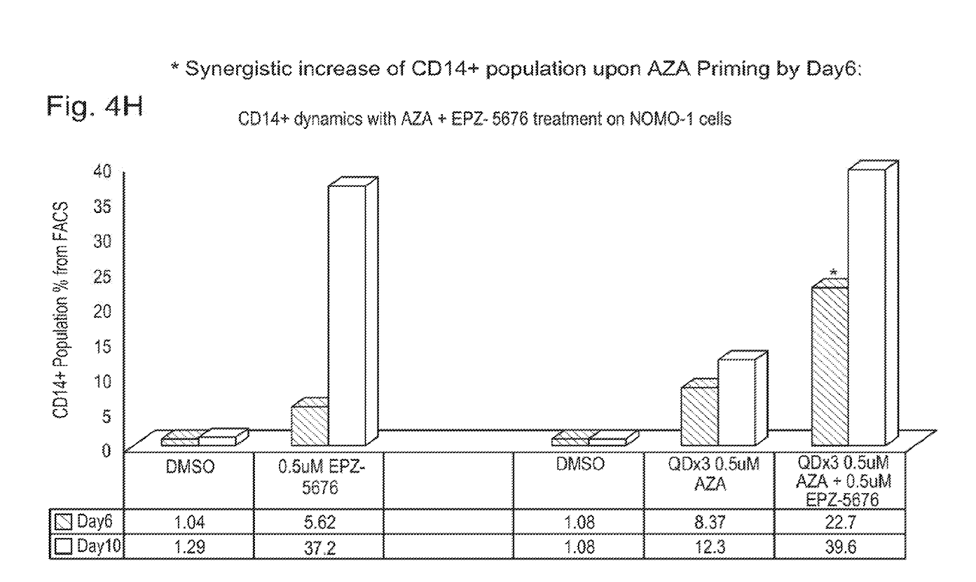

These data also indicate that AZA priming leads to decrease in MEIS1 RNA levels after Day 2 but does not sustain low levels in later time points (see FIG. 4). This decrease in MEIS RNA levels after Day 2 may be sufficient to sensitize the cells towards enhanced cell death with AZA priming (i.e. see the CTG assay results, Annexin V, cell count and Incucyte data in FIGS. 1-16). Moreover, in the EPZ-5676 and AZA resistant cell line NOMO-1, there is synergy for differentiation by Day 6 as seen by the CD14+ population increase with AZA priming followed by EPZ-5676. Collectively, the data presented herein indicate that EPZ-5676 treatment reduces MEIS1 and HOXA9 expression in a dose dependent manner, as well as reduces the H3K79me2 mark on MEIS1 and HOXA9 genes as demonstrated in ChIP-qPCR assays (see FIG. 3).

The results for the differentiation assays performed on the MV4-11, NOMO-1 and ML-2 & RS4;11 cell lines are summarized in Table 1 below and in FIGS. 1-4.

TABLE 1
Results of Cellular Differentiation Assay

| AML Cell Line | Day 6 | Day 10 |
|---|---|---|
| MV4-11 | Increased cell kill with | No increase in cell kill |
| | AZA priming | with AZA priming due to |
| | Depletion of CD11b+ cell | decrease in cell viability |
| | population with and | with time |
| | without AZA priming | Depletion of CD11b+ |
| | No increase in CD14+ | cell population with and |
| | cell population | without AZA priming |
| | | No increase in CD14+_{—} |
| | | cell population |
| NOMO-1 | No cell kill with EPZ- | No cell kill with EPZ- |
| | 5676 alone or AZA priming | 5676 alone or AZA priming |
| | Synergy in CD14+ cell | Similar CD14+ cell |
| | population increase upon | population levels with |
| | AZA priming | and without AZA priming |
| ML-2 &RS4; | No cell kill with EPZ- | No cell kill with EPZ- |
| 11 | 5676 alone and AZA | 5676 alone and AZA |
| | effect in cell kill for | effect in cell kill for |
| | combination | combination |
| | No differentiation | No differentiation |

The influence of the AZA priming followed by EPZ-5676 on the AML cell lines is detailed in Table 2 below.

TABLE 2
Influence of AZA Priming Followed
by EPZ-5676 in AML Cell Lines

| AML Cell Line | Influence of Treatment Conditions |
|---|---|
| MV4-11 (MLL-AF4) | AZA priming + EPZ-5676 results in enhanced |
| | cell kill compared to EPZ-5676 alone |
| | AZA priming + EPZ-5676 leads to rapid |
| | reduction of MEIS1 RNA levels compared |
| | to EPZ-5676 alone on Day 2 but not |
| | sustained through Day 9 |
| | AZA priming + EPZ-5676 and EPZ-5676 |
| | single agent both decrease H3K79me2 |
| | mark in ChIP-qPCR assays |
| | No differentiation effect seen with and |
| | without AZA priming |
| | EPZ-5676 does not induce cell death in |
| | MV4-11 cells in a dose response experiment |
| | AZA induces cell death in MV4-11 cells |
| | in a dose response experiment |
| | 3 day AZA primed (0.3 μM and 1 μM) |
| | MV4-11 followed by EPZ-5676 treatment |
| | displays synergy |
| NOMO-1 (MLL-AF9) | AZA priming + EPZ-5676 leads to |
| | synergistic increase in CD14+ |
| | differentiated cell population compared to |
| | EPZ-5676 alone by Day 6 |
| ML-2 (MLL-AF6) | AZA priming + EPZ-5676 leads to slight |
| | increase in CD14 + differentiated cell |
| | population as compared to no |
| | differentiation with EPZ-5676 alone |
| RS4; 11 (MLL-AF4) | AZA priming + EPZ-5676 leads to |
| | increased cell kill as compared to EPZ- |
| | 5676 alone |
| THP-1 | No synergy to EPZ-5676 upon AZA priming |

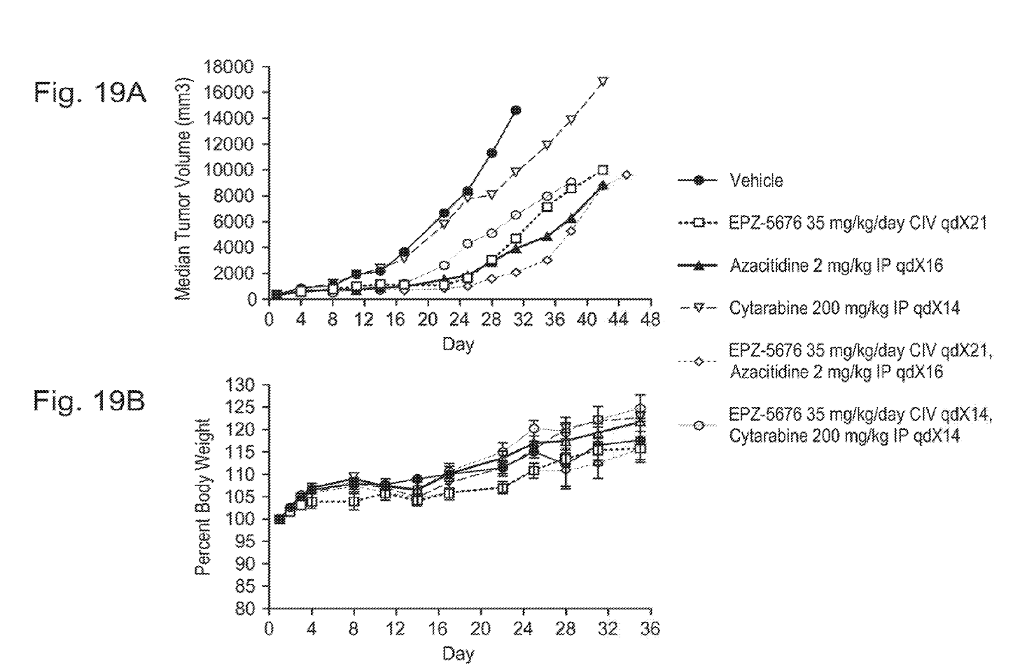

To follow up on these findings and to determine if combinations were tolerable and efficacious in vivo, nude rats implanted subcutaneously with MV4-11 tumors were dosed using a range of doses and schedules. The maximum tolerated dose (MTD) for Azacitidine and Cytarabine were 2 and 200 mg/kg respectively, and delivered by intraperitoneal injection once daily for 14 days. In preparation for an efficacy combination study, the Azacitidine and Cytarabine single agent activity in a MV4-11 model was determined. Dosing at the established MTD, it was found that these agents inhibited the subcutaneous MV4-11 tumor by 50% compared to its vehicle control. Combination efficacy results from the EPZ-5676 combination studies with Cytarabine or Azacitidine are presented in FIGS. 19.

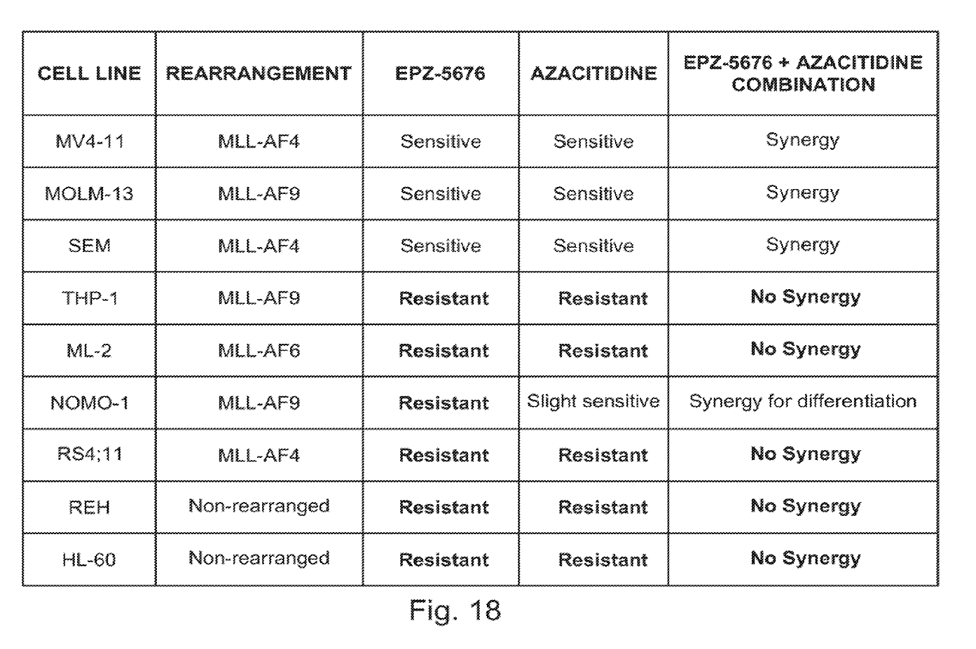

FIG. 18 demonstrates that synergistic cell killing with EPZ-5676 is achieved in MLL-rearranged leukemia cell lines sensitive to both EPZ-5676 and Azacitidine. Compounds were evaluated for cell killing effects as single agents and in combination according to the methods described in the experimental setup. Using the Annexin V/7-AAD flow cytometry assay, cell lines are defined as sensitive to Azacitidine, EPZ-5676 or their combination when dose response curves can be fitted to a 4-parameter logistic curve and the derived IC₅₀ value is less or equal to 3 μM.

FIG. 19 demonstrates results from an MV4-11 efficacy study with EPZ-5676 treated in combination with Azacitidine or Cytarabine. EPZ-5676 sensitive MLL-r subcutaneous nude rat model MV4-11 were exposed to a combination of EPZ-5676 with either Azacitidine or Cytarabine. 35 mg/kg/day EPZ-5676 was administered by continous IV (CIV) infusion while Azacitdine and Cytarabine was dosed daily by intraperitoneal injection at 2 and 200 mg/kg respectively. It was observed that combination treatment with Azacitidine shows signficant advantage over treatment with the individual therapies only at the day 36 time point (Repeated measures ANOVA with Dunnett post test, P value=0.028). Time points including Days 28, 32, 38 and 42 do not reach significance (P value≦0.05). The tumor volumes are represented as the median tumor volumes mm³ of the group. No significant body weight loss was observed in either the single agent or combination treatments.

In summary, EPZ-5676 in combination with Cytarabine or Azacitidine revealed a synergistic effect regardless of the treatment schedule used in preclinical models of AML MLL-r leukemia. Tolerable in vivo rat combination doses for EPZ-5676 with both Cytarabine and Azacitidine have been determined in support of potential future assessment of these combinations in the leukemia patient populations containing MLL-r.

Combination benefit was achieved with Azacitidine priming followed by treatment with Compound A2 alone or in combination with Azacitidine in the following tested in MLL-rearranged AML cell lines: Molm-13 (MLL-AF9) and MV4-11 (MLL-AF4) and SEM (MLL-AF4).

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A combination comprising a DNA methyltransferase (DNMT) inhibitor and Compound A2 or a pharmaceutically acceptable salt thereof.

2. The combination of claim 1, wherein the DNMT inhibitor is Azacitidine, Decitabine, or Zebularine.

3. The combination of claim 1, wherein the DNMT inhibitor is Azacitidine.

4. A method of treating cancer comprising administering to a subject in need thereof a therapeutically effective amount of a DNA methyltransferase (DNMT) inhibitor, prior to administering a therapeutically effective amount of Compound A2 or a pharmaceutically acceptable salt thereof.

5. A method of treating cancer comprising administering to a subject in need thereof a therapeutically effective amount of a DNA methyltransferase (DNMT) inhibitor, wherein the therapeutically effective amount is an amount sufficient to sensitize the subject to a subsequent treatment with Compound A2 or a pharmaceutically acceptable salt thereof.

6. The method of claim 5, further comprising administering to the sensitized subject a therapeutically effective amount of Compound A2 or a pharmaceutically acceptable salt thereof.

7. The method of any one of claims 4-6, wherein multiple doses of a DNMT inhibitor are administered prior to administration of Compound A2.

8. The method of any one of claims 4-7, wherein Compound A2 or a pharmaceutically acceptable salt thereof is administered at least one, two, three or more hours following the administration of the DNMT inhibitor.

9. The method of any one of claims 4-7, wherein Compound A2 or a pharmaceutically acceptable salt thereof is administered at least one, two, three or more days following the administration of the DNMT inhibitor.

10. A method of treating cancer comprising administering to a subject in need thereof a therapeutically effective amount of a DNA methyltransferase (DNMT) inhibitor, prior to administering a therapeutically effective amount of the combination of any one of claims 1-3.

11. A method of treating cancer comprising administering to a subject in need thereof a therapeutically effective amount of a DNA methyltransferase (DNMT) inhibitor, wherein the therapeutically effective amount is an amount sufficient to sensitize the subject to a subsequent treatment with the combination of any one of claims 1-3.

12. The method of claim 11, further comprising administering to the sensitized subject a therapeutically effective amount of the combination.

13. The method of any one of claims 10-12, wherein multiple doses of a DNA DNMT inhibitor are administered prior to administration of the combination.

14. The method of any one of claims 10-13, wherein the combination is administered at least one, two, three or more hours following the administration of the DNMT inhibitor.

15. The method of any one of claims 10-13, wherein the combination is administered at least one, two, three or more days following the administration of the DNMT inhibitor.

16. The method of any one of claims 5-9 and 11-15, wherein the sensitization is determined by the methylation status of histones or other proteins.

17. The method of any one of claims 5-9 and 11-15, wherein the sensitization is determined by a decreased level of methylation of histones or other proteins, wherein the level is decreased compared to a non-sensitized subject.

18. The method of any one of claims 5-8 and 10-13, wherein the sensitization is determined by decreased level of methylation of H3K79.

19. The method of any one of claims 5-9, wherein the amount of Compound A2 or a pharmaceutically acceptable salt thereof that is therapeutically effective is smaller than the amount of Compounds A2 that is therapeutically effective in a subject that was not sensitized with the DNMT inhibitor.

20. The method of any one of claims 11-15, wherein the amount of the combination that is therapeutically effective is smaller than the amount of the combination that is therapeutically effective in a subject that was not sensitized with the DNMT inhibitor.

21. The method of any one of claims 10-15 and 20, wherein the combination is administered at a dosage of 0.01 mg/kg per day to about 1000 mg/kg per day.

22. The method of any one of claims 10-15 and 20-21, wherein the DNMT inhibitor and Compound A2 of the combination are delivered simultaneously, sequentially, or in alternation.

23. The method of any one of claims 4-9 and 19, wherein Compound A2 or a pharmaceutically acceptable salt thereof is administered at a dosage of 0.01 mg/kg per day to about 1000 mg/kg per day.

24. The method of any one of claims 4-9 and 19, wherein Compound A2 or a pharmaceutically acceptable salt thereof is administered at a dose of at least 12, 24, 36, 45, 54, 70, 80, or 90 mg/m²/day.

25. The method of any one of claims 4-9, 19, and 23-24, wherein Compound A2 or a pharmaceutically acceptable salt thereof is administered continuously for at least 4, 5, 6, 7, 14, 21, 28, 35, 42, 47, 56, or 64 days.

26. The method of claim 25, wherein continuous administration comprises administration without a drug holiday.

27. The method of any one of claims 4-26, wherein the DNMT inhibitor is administered at a dosage of 0.01 mg/kg per day to about 1000 mg/kg per day.

28. The method of any one of claims 4-27, wherein the DNMT inhibitor is Azacitidine, Decitabine, or Zebularine.

29. The method of any one of claims 4-27, wherein the DNMT inhibitor is Azacitidine.

30. The method of any one of claims 4-29, wherein the administration results in maturation or differentiation of leukemic blast cells.

31. The method of claim 30 wherein at least 20% of leukemic blast cells have undergone maturation or differentiation.

32. The method of claim 30, wherein at least 50% of leukemic blast cells have undergone maturation or differentiation.

33. The method of claim 30, wherein at least 80% of leukemic blast cells have undergone maturation or differentiation.

34. The method of any one of claims 4-33, wherein administration results in reduction of H3K79 methyl mark to at least 90%, 80%, 70%, 60%, 50%, 40%, 30%, 20%, 10% or less of untreated control levels.

35. The method of any one of claims 4-34, wherein administration results in the suppression of H3K79 methyl mark rebound.

36. The method of any one of claims 4-35, wherein administration results in at least 20%, 30%, 40%, 50%, 60%, 70%, 80%, or 90% of leukemic blast cells undergoing cell death or apoptosis.

37. The method of any one of claims 4-36, wherein the method of treatment includes resolution of fevers, resolution of cachexia or resolution of leukemia cutis.

38. The method of any one of claims 4-37, wherein the method of treatment includes restoration of normal haematopoiesis.

39. The method of any one of claims 4-38, wherein the subject has demonstrated resistance to any one of the components of the combination of any one of claims 1-3 when administered as a single agent.

40. The method of any one of claims 4-39, wherein the subject is a pediatric patient aged 3 months to 18 years.

41. The method of any one of claims 4-40, wherein the subject has leukemia.

42. The method of claim 41, wherein the leukemia is characterized by a chromosomal rearrangement.

43. The method of claim 42, wherein the chromosomal rearrangement is chimeric fusion of mixed lineage leukemia gene (MLL) or partial tandem duplication of MLL (MLL-PTD).

44. The method of any one of claims 4-43, wherein the subject has an increased level of HOXA9, Fms-like tyrosine kinase 3 (FLT3), MEIS1, and/or DOT1L.

45. A method of inhibiting cancer cell proliferation comprising contacting a cancer cell with an effective amount of a DNA methyltransferase (DNMT) inhibitor, prior to administering an effective amount of Compound A2, or a pharmaceutically acceptable salt thereof.

46. A method of inhibiting cancer cell proliferation comprising contacting a cancer cell with an effective amount of a DNA methyltransferase (DNMT) inhibitor, prior to administering an effective amount of the combination of any one of claims 1-3.

47. The method of claim 45 or 46, wherein the effective amount of the DNMT inhibitor is at least 0.01 μM.

48. The method of claim 45 or 46, wherein the effective amount of the DNMT inhibitor is at least 0.1 μM.

49. The method of claim 45 or 46, wherein the effective amount of the DNMT inhibitor is at least 0.3 μM.

50. The method of any one of claim 45 or 47-49, wherein multiple doses of a DNMT inhibitor are administered prior to administration of Compound A2

51. The method of any one of claims 46-49, wherein multiple doses of a DNA methyltransferase (DNMT) inhibitor are administered prior to administration of the combination.

52. The method of any one of claims 45-51, wherein the DNMT inhibitor is Azacitidine, Decitabine, or Zebularine.

53. The method of any one of claims 45-51, wherein the DNMT inhibitor is Azacitidine.
